(19) United States
(12) Patent Application Publication
(10) Pub. No.: US 2024/0363252 A1
(43) Pub. Date: Oct. 31, 2024

(54) Determining Visual Frailty Index Using Machine Learning Models

(71) Applicant: The Jackson Laboratory, Bar Harbor, ME (US)

(72) Inventors: Vivek Kumar, Bar Harbor, ME (US); Leinani Hession, Bar Harbor, ME (US); Gautam Sabnis, Bar Harbor, ME (US); Gary Churchill, Bar Harbor, ME (US)

(21) Appl. No.: 18/560,081

(22) PCT Filed: May 12, 2022
(86) PCT No.: PCT/US2022/028986

§ 371 Date: Nov. 9, 2023

Related U.S. Application Data
(60) Provisional application No. 63/187,892, filed on May 12, 2021.

(51) Int. Cl. G16H 50/30; G06T 7/00; G16H 30/40

(52) U.S. Cl. CPC G16H 50/30; G06T 7/0016; G16H 30/40; G06T 2207/20081; G06T 2207/20084; G06T 2207/30012

(57) ABSTRACT

Systems and methods described herein provide techniques for determining a visual frailty score by processing video data of a subject. Various features maybe used to determine the visual frailty score, including but not limited to, spinal mobility features, gait measurements, behavior features, and body composition data. The various features may be extracted from the video data using different techniques. The various features may be processed using one or more machine learning models to determine the visual frailty score.

### RELATED APPLICATIONS

This application claims benefit under 35 U.S.C § 119(e) of U.S. Provisional application Ser. No. 63/187,892 filed May 12, 2021, the disclosure of which is incorporated by reference herein in its entirety.

### GOVERNMENT SUPPORT

This invention was made with government support under DA041668 and DA048634 awarded by National Institute of Drug Abuse and AG38070 awarded by National Institute of Aging. The government has certain rights in the invention.

### FIELD OF THE INVENTION

The invention, in some aspects, relates to determining a visual frailty index of a subject by processing video data using machine learning models.

### BACKGROUND

Aging is a terminal process that affects all biological systems. Biological aging—in contrast to chronological aging—occurs at different rates for different individuals. In humans, growing old comes with increased health issues and mortality rates, yet some individuals live long and healthy lives while others succumb earlier to diseases and disorders. More precisely, there is an observed heterogeneity in mortality risk and health status among individuals within an age cohort [Mitnitski, A., et al., The Scientific World Journal 1, 323-36 (September 2001); Whitehead, J. C. et al. Journals of Gerontology Series A: Biomedical Sciences and Medical Sciences 69, 621-632 (2014)]. The concept of frailty is used to quantify this phenomenon of heterogeneity and is defined as the state of increased vulnerability to adverse health outcomes [Rockwood, K., et al. CMAJ 150, 489-495 (1994)]. Identifying frailty is clinically important as frail individuals have increased risk of diseases and disorders, worse health outcomes from the same disease, and even different symptoms of the same disease [Whitehead, J. C. et al. Journals of Gerontology Series A: Biomedical Sciences and Medical Sciences 69, 621-632 (2014)].

Frailty index (FI) is a widely used approach to quantify frailty [Mitnitski, A., et al., The Scientific World Journal 1, 323-36 (September 2001)] and outperforms other methods [Schultz, M. B. et al. Nature Communications 11, 1-12 (2020)]. In this method, an individual is scored on a set of age-related health deficits to produce a cumulative score. Each deficit must have the following characteristics: they must be health related, they must increase in the population with age, and they must not saturate in the population too early [Searle, S. D., et al., BMC geriatrics 8, 24 (2008)]. The presence and severity of each health deficit is scored as 0 for not present, 0.5 for partially present, or 1 for present. A compelling finding of FIs is that the exact health deficits scored can vary between indexes but still show similar characteristics and utility [Searle, S. D., et al., BMC geriatrics 8, 24 (2008).]. That is, two sufficiently large FIs with a different number and selection of deficits scored would still show a similar average rate of deficit accumulation with age and the same submaximal limit of possible FI score. More importantly, both FIs would strongly predict an individual's risk of adverse health outcomes, hospitalization, and mortality. This attribute of FIs is advantageous as researchers can pull data from varied large health databases, aiding in large-scale studies. It also suggests that frailty is a legitimate phenomenon and that FIs are a valid way of quantifying it, given the complexity of aging. Different people age not only at different rates but in different ways; one person may have severe mobility issues but have a sharp memory, while another may have a healthy heart but a weak immune system. Both may be equally frail, but this is only made clear by sampling a variety of health deficits. Indeed, FI scores outperform other developed measures like tracking molecular markers, and frailty phenotyping at efficiently predicting mortality risk and health status [Schultz, M. B. et al. Nature Communications 11, 1-12 (2020); Kim, S., et al., GeroScience 39, 83-92 (January 2017); and Kojima, G., et al., Age and Ageing 47, 193-200. (2017)]. Some FIs have been adapted for use in mice using a variety of both behavioral and physiological measures as index items [Whitehead, J. C. et al. Journals of Gerontology Series A: Biomedical Sciences and Medical Sciences 69, 621-632 (2014); Schultz, M. B. et al. Nature Communications 11, 1-12 (2020); and Parks, R. et al. The journals of gerontology. Series A, Biological sciences and medical sciences 67, 217-27 (March 2012)], but there exists a lack of adequate methods for assessing frailty and predicting mortality risk and health in animal models and in humans.

### SUMMARY OF THE INVENTION

According to an aspect of the invention a computer-implemented method is provided, the method including receiving video data representing a video capturing movements of a subject; determining, using the video data, spinal mobility features of the subject for a duration of the video; and processing, using at least one machine learning model, at least the spinal mobility features to determine a visual frailty score for the subject. In some embodiments, determining the spinal mobility features of the subject for the duration of the video includes: determining a plurality of spinal measurements, each spinal measurement of the plurality of spinal measurements corresponding to one video frame of the video data; and determining the spinal mobility features using the plurality of spinal measurements. In some embodiments, determining the spinal mobility features of the subject for the duration of the video includes: for each video frame of the video data: determining a first distance between a head of the subject and a tail of the subject; determining a second distance between a mid-back of the subject and a midpoint between the head and the tail; determining an angle formed between the head, the tail and the mid-back of the subject; and determining the spinal mobility features for a video frame to include the first distance, the second distance and the angle. In some embodiments, determining the spinal mobility features of the subject for the duration of the video includes: determining, for each video frame of the video data, a distance between a mid-back of the subject and a midpoint between a head of the subject and a tail of the subject. In some embodiments, the method also includes processing, using at least an additional machine learning model, the video data to determine pose estimation data tracking, during the duration of the video, a location of at least a head of the subject, a tail of the subject, and a mid-back of the subject; and using the pose estimation data to determine the spinal mobility features. In some embodiments, the method also includes processing the video data to determine pose estimation data tracking, during the duration of the video, a location of at least twelve body parts of the subject; determining, using the pose estimation data, features for the subject; and processing, using the at least one machine learning model, the features to determine the visual frailty score. In some embodiments, the method also includes determining body features for the subject, the body features corresponding to at least one of a length of the subject, a width of the subject, and a distance between rear paws of the subject; processing, using the at least one machine learning model, the body features to determine the visual frailty score. In some embodiments, the method also includes determining a number of times a rearing event occurs during the duration of the video; determining a rearing length for each rearing event; and processing, using the at least one machine learning model, the number times the rearing event occurs and the rearing length for each rearing event to determine the visual frailty score. In some embodiments, the method also includes processing, using the at least one machine learning model, the video data to determine ellipse-fit data for the subject for the duration of the video; determining, using the ellipse-fit data, features for the subject; and processing, using the at least one machine learning model, the features to determine the visual frailty score. In some embodiments, determining spinal mobility features of the subject for a duration of the video includes: determining a first set of video frames representing gait movements by the subject; determining a first set of spinal mobility features for the first set of video frames; determining a second set of video frames representing non-gait movements by the subject; and determining a second set of spinal mobility features for the second set of video frames; wherein the spinal mobility features include the first set of spinal mobility features and the second set of spinal mobility features. In some embodiments, the first set of spinal mobility features correspond to a distance between a mid-back of the subject and a midpoint between a head and a tail of the subject, and wherein the second set of spinal mobility features correspond to an angle formed between the head, the tail and the mid-back of the subject. In some embodiments, the method also includes determining, using the video data, gait measurements of the subject for the duration of the video; and processing, using the at least one machine learning model, the gait features to determine the visual frailty score for the subject. In some embodiments, the method also includes processing the video data to determine point data tracking movement, for the duration of the video, of a set of body parts of the subject; determining, using the point data, a plurality of stance phases and a plurality of swing phases represented in the video data; determining, based on the plurality of stance phases and the plurality of swing phases, a plurality of stride intervals represented in the video data; and determining, using the point data, the gait measurements based on each stride interval of the plurality of stride intervals. In some embodiments, the method also includes determining a first transition from a first stance phase of the plurality of stance phases and a first swing phase of the plurality of swing phases based on a toe-off event of a left hind paw of the subject or a right hind paw of the subject; determining a second transition from a second swing phase of the plurality of swing phases to a second stance phase of the plurality of stance phases based on a foot strike event of the left hind paw or the right hind paw; and determining the gait measurements using the first transition and the second transition. In some embodiments, the method also includes processing the video data to determine point data tracking movement, for the duration of the video, of a set of body parts of the subject, wherein the set of body parts comprises a left hind paw and a right hind paw, and wherein determining the gait measurements comprises: determining, using the point data, a step length for a stride interval, the step length representing a distance that the right hind paw travels past a previous left hind paw strike; determining, using the point data, a stride length using for the stride interval, the stride length representing a distance that the left hind paw travels during the each stride interval; and determining, using the point data, a step width for the stride interval, the step width representing a distance between the left hind paw and the right hind paw. In some embodiments, the method also includes processing the video data to determine point data tracking movement, for the duration of the video, of a set of body parts of the subject, wherein the set of body parts comprises a tail base, and wherein determining the gait measurements comprises determining, using the point data, speed data of the subject based on movement of the tail base for a stride interval. In some embodiments, the method also includes processing the video data to determine point data tracking movement, for the duration of the video, of a set of body parts of the subject, wherein the set of body parts comprises a tail base, and wherein determining the gait measurements includes: determining, using the point data, a set of speed data of the subject based on movement of the tail base during a set of frames representing a stride interval of the plurality of stride intervals; and determining a stride speed, for the stride interval, by averaging the set of speed data. In some embodiments, the method also includes: processing the video data to determine point data tracking movement, for the duration of the video, of a set of body parts of the subject, wherein the set of body parts comprises a right hind paw and a left hind paw, and wherein determining the gait measurements includes: determining, using the point data, first stance duration representing an amount of time that the right hind paw is in contact with ground during a stride interval; determining a first duty factor based on the first stance duration and the duration of the stride interval; determining, using the point data, second stance duration representing an amount of time that the left hind paw is in contact with ground during the stride interval; determining a second duty factor based on the second stance duration and the duration of the stride interval; and determining an average duty factor for the stride interval based on the first duty factor and the second duty factor. In some embodiments, the method also includes processing the video data to determine point data tracking movement, for the duration of the video, of a set of body parts of the subject, wherein the set of body parts comprises a tail base and a neck base, and wherein determining the gait measurements includes: determining, using the point data, a set of vectors connecting the tail base and the neck base during a set of frames representing a stride interval of the plurality of stride intervals; and determining, using the set of vectors, an angular velocity of the subject for the stride interval. In some embodiments, the method also includes processing the video data to determine point data tracking movement, for the duration of the video, of a set of body parts of the subject, wherein the set of body parts comprises a spine center of the subject, wherein a stride interval is associated with a set of frames of the video data, and wherein determining the gait measurements comprises determining, using the point data, a displacement vector for the stride interval, the displacement vector connecting the spine center represented in a first frame of the set of frames and the spine center represented in a last frame of the set of frames. In some embodiments, the set of body parts also includes a nose of the subject, and wherein determining the metrics data includes: determining, using the point data, a set of lateral displacements of the nose for the stride interval based on a perpendicular distance of the nose from the displacement vector for each frame in the set of frames. In some embodiments, the lateral displacement of the nose is further based on a body length of the subject. In some embodiments, determining the gait measurements also includes determining a tail tip displacement phase offset by: performing an interpolation using the set of lateral displacements of the nose to generate a smooth curve lateral displacement of the nose for the stride interval; determining, using the smooth curve lateral displacement of the nose, when a maximum displacement of the nose occurs during the stride interval; and determining a percent stride location representing a percent of the stride interval that is completed when the maximum displacement of the nose occurs. In some embodiments, the method also includes processing the video data to determine point data tracking movement, for the duration of the video, of a set of body parts of the subject, wherein the set of body parts further comprises a tail base of the subject, and wherein determining the gait measurements comprises determining, using the point data, a set of lateral displacements of the tail base for the stride interval based on a perpendicular distance of the tail base from the displacement vector for each frame in the set of frames. In some embodiments, determining the gait measurements further comprises determining a tail base displacement phase offset by: performing an interpolation using the set of lateral displacements of the tail base to generate a smooth curve lateral displacement of the tail base for the stride interval; determining, using the smooth curve lateral displacement of the tail base, when a maximum displacement of the tail base occurs during the stride interval; and determining a percent stride location representing a percent of the stride interval that is completed when the maximum displacement of the tail base occurs. In some embodiments, the method also includes processing the video data to determine point data tracking movement, for the duration of the video, of a set of body parts of the subject, wherein the set of body parts comprises a tail tip of the subject, and wherein determining the gait measurements includes: determining, using the point data, a set of lateral displacements of the tail tip for the stride interval based on a perpendicular distance of the tail tip from the displacement vector for each frame in the set of frames. In some embodiments, determining the gait measurements also includes determining a tail tip displacement phase offset by: performing an interpolation using the set of lateral displacements of the tail tip to generate a smooth curve lateral displacement of the tail tip for the stride interval; determining, using the smooth curve lateral displacement of the tail tip, when a maximum displacement of the tail tip occurs during the stride interval; and determining a percent stride location representing a percent of the stride interval that is completed when the maximum displacement of the tail tip occurs. In some embodiments, the method also includes processing the video data to determine point data tracking movement, for the duration of the video, of a set of body parts, wherein the set of body parts comprises one or more of: the nose, base of neck, mid spine, left hind paw, right hind paw, base of tail, middle of tail and tip of tail; determining, using the point data, features for the subject; and processing, using at least the one machine learning model, the features to determine the visual frailty score. In some embodiments, the method also includes processing the video data using an additional machine learning model to identify a likelihood of the subject exhibiting a grooming behavior for a plurality of video frames of the video data; and determining the visual frailty score using the likelihood of the subject exhibiting the grooming behavior. In some embodiments, the method also includes processing the video data using an additional machine learning model to identify a likelihood of the subject exhibiting a predetermined behavior for a plurality of video frames of the video data; and determining the visual frailty score using the likelihood of the subject exhibiting the predetermined behavior. In some embodiments, the method also includes determining a rotated set of video frames by rotating a first set of video frames of the video data; processing the first set of video frames using a first machine learning model configured to identify a likelihood of the subject exhibiting a predetermined behavioral action; based on the processing of the first set of video frames by the first machine learning model, determining a first probability of the subject exhibiting the predetermined behavioral action in a first video frame of the first set of video frames, the first video frame corresponding to a first duration of the video data; processing the rotated set of frames using the first machine learning model; based on the processing of the rotated set of video frames by the first machine learning model, determining a second probability of the subject exhibiting the predetermined behavioral action in a second video frame of the rotated set of video frames, the second video frame corresponding to the first duration of the video data; and using the first probability and the second probability, identifying a first label for the first video frame, the first label indicating that the subject exhibits the predetermined behavioral action. In some embodiments, the method also includes processing the first set of video frames using a second machine learning model configured to identify a likelihood of the subject exhibiting the predetermined behavioral action; based on the processing of the first set of video frames by the second machine learning model, determining a third probability of the subject exhibiting the predetermined behavioral action in the first video frame; processing the rotated set of video frames using the second machine learning model; based on the processing of the rotated set of video frames by the second machine learning model, determining a fourth probability of the subject exhibiting the predetermined behavioral action in the second video frame; and identifying the first label using the first probability, the second probability, the third probability and the fourth probability. In some embodiments, the method also includes determining a reflected set of video frames by reflecting the first set of video frames; processing the reflected set of video frames using the first machine learning model; based on the processing of the reflected set of video frames by the first machine learning model, determining a third probability of the subject exhibiting the predetermined behavioral action in a third video frame of the reflected set of frames, the third video frame corresponding to the first duration of the first video frame; and identifying the first label using the first probability, the second probability, and the third probability. In some embodiments, the subject is a mouse, and the predetermined behavior comprises a grooming behavior comprising at least one of paw licking, unilateral face wash, bilateral face wash, and flank licking. In some embodiments, the first set of video frames represent a portion of the video data during a time period, and the first video frame is a last temporal frame of the time period. In some embodiments, the method also includes identifying a second set of video frames from the video data; determining a second rotated set of video frames by rotating the second set of video frames; processing the second set of video frames using the first machine learning model; based on the processing of the second set of video frames by the first machine learning model, determining a third probability of the subject exhibiting the predetermined behavioral action in a third video frame of the second set of video frames; processing the second rotated set of video frames using the first machine learning model; based on the processing of the second rotated set of video frames by the first machine learning model, determining a fourth probability of the subject exhibiting the predetermined behavioral action in a fourth video frame of the rotated set of frames, the fourth video frame corresponding to the third video frame; and using the third probability and the fourth probability, identifying a second label for the fourth video frame, the second label indicating that the subject exhibits the predetermined behavioral action. In some embodiments, the first machine learning model is a machine learning classifier. In some embodiments, the method also includes processing the video data to determine gait measurements for the subject for the duration of the video; processing the video data to determine behavior data identifying portions of the video where the subject exhibits a predetermined behavior; and processing, using the at least one machine learning model, the spinal mobility features, the gait measurements and the behavior data to determine the visual frailty score. In some embodiments, the video captures movements of the subject in an open field arena. In some embodiments, the method also includes determining a physical condition of the subject using the visual frailty score. In some embodiments, the physical condition is frailty. In some embodiments, the frailty is a symptom of a disease or condition. In some embodiments, the physical condition is a pre-frailty condition. In some embodiments, the subject is a mammal, optionally a mouse.

According to another aspect of the invention, a method of assessing a physical condition of a subject is provided, the method including determining a visual frailty score for the subject with the computer-implemented method of any embodiment of any one of the aforementioned aspects. In some embodiments, the physical condition is frailty. In some embodiments, the physical condition is a pre-frailty condition. In some embodiments, the physical condition is a symptom of a disease or condition. In some embodiments, the subject is a mammal, optionally a mouse.

According to another aspect of the invention, a method of determining the presence of an effect of a candidate compound on a frailty condition is provided, the method including: obtaining a first visual frailty score for a subject, wherein a means for the obtaining comprises a computer-implemented method of any one of claims A1-A39, and wherein the subject has a frailty condition or is an animal model for the frailty condition; administering to the subject the candidate compound; obtaining a post-administration visual frailty score for the subject; comparing the first and the post-administration visual frailty score, wherein a difference in the first and post-administration visual frailty score identifies an effect of the candidate compound on the frailty condition. In some embodiments, an improvement in the visual frailty score indicating less frailty identifies the candidate compound as enhancing regression of the frailty condition. In some embodiments, a post-administration visual frailty score that is statistically equivalent to the first visual frailty score identifies the candidate compound as inhibiting progression of the frailty condition in the subject. In some embodiments, the method also includes additional testing of the compound's effect in treatment of the frailty condition. In some embodiments, the subject is a mammal, optionally a mouse.

According to another aspect of the invention, a method of identifying the presence of an effect of a candidate compound on a frailty condition is provided, the method including: administering the candidate compound to a subject that has the frailty condition or that is an animal model for the frailty condition; obtaining a visual frailty score for the subject, wherein a means for the obtaining comprises an embodiment of a computer-implemented method of any aforementioned aspect of the invention; comparing the obtained visual frailty score to a control visual frailty score, wherein a difference in the obtained visual frailty score and the control visual frailty score identifies the presence of an effect of the candidate compound on the frailty condition. In some embodiments, an improvement in the visual frailty score indicating less frailty in the subject administered the candidate compound compared to the control frailty score identifies the candidate compound as enhancing regression of the frailty condition in the subject. In some embodiments, a visual frailty score obtained in the subject administered the candidate compound that is statistically equivalent to the control frailty score identifies the candidate compound as inhibiting progression of the frailty condition in the subject. In some embodiments, the subject is a mammal, optionally a mouse.

According to another aspect of the invention, a system is provided, the system including: at least one processor; and at least one memory comprising instructions that, when executed by the at least one processor, cause the system to: receive video data representing a video capturing movements of a subject; determine, using the video data, spinal mobility features of the subject for a duration of the video; and process, using at least one machine learning model, at least the spinal mobility features to determine a visual frailty score for the subject. In some embodiments, the instructions that cause the system to determine the spinal mobility features of the subject for the duration of the video further cause the system to: determine a plurality of spinal measurements, each spinal measurement of the plurality of spinal measurements corresponding to one video frame of the video data; and determine the spinal mobility features using the plurality of spinal measurements. In some embodiments, the instructions that cause the system to determine the spinal mobility features of the subject for the duration of the video further cause the system to: for each video frame of the video data: determine a first distance between a head of the subject and a tail of the subject; determine a second distance between a mid-back of the subject and a midpoint between the head and the tail; determine an angle formed between the head, the tail and the mid-back of the subject; and determine the spinal mobility features for a video frame to include the first distance, the second distance and the angle. In some embodiments, the instructions that cause the system to determine the spinal mobility features of the subject for the duration of the video further cause the system to: determine, for each video frame of the video data, a distance between a mid-back of the subject and a midpoint between ahead of the subject and a tail of the subject. In some embodiments, the at least one memory includes further instructions, that when executed by the at least one processor, cause the system to: process, using the at least one machine learning model, the video data to determine pose estimation data tracking, during the duration of the video, a location of at least a head of the subject, a tail of the subject, and a mid-back of the subject; and use the pose estimation data to determine the spinal mobility features. In some embodiments, the at least one memory includes further instructions, that when executed by the at least one processor, cause the system to: process the video data to determine pose estimation data tracking, during the duration of the video, a location of at least twelve body parts of the subject; determine, using the pose estimation data, features for the subject; and process, using the at least one machine learning model, the features to determine the visual frailty score. In some embodiments, at least one memory includes further instructions, that when executed by the at least one processor, cause the system to: determine body features for the subject, the body features corresponding to at least one of a length of the subject, a width of the subject, and a distance between rear paws of the subject; process, using the at least one machine learning model, the body features to determine the visual frailty score. In some embodiments, the at least one memory includes further instructions, that when executed by the at least one processor, cause the system to: determine a number of times a rearing event occurs during the duration of the video; determine a rearing length for each rearing event; process, using the at least one machine learning model, the number times the rearing event occurs and the rearing length for each rearing event to determine the visual frailty score. In some embodiments, the at least one memory includes further instructions, that when executed by the at least one processor, cause the system to: process, using at least an additional machine learning model, the video data to determine ellipse-fit data for the subject for the duration of the video; determine, using the ellipse-fit data, features for the subject; and process, using the at least one machine learning model, the features to determine the visual frailty score. In some embodiments, the instructions that cause the system to determine the spinal mobility features of the subject for the duration of the video further cause the system to: determine a first set of video frames representing gait movements by the subject; determine a first set of spinal mobility features for the first set of video frames; determine a second set of video frames representing non-gait movements by the subject; and determine a second set of spinal mobility features for the second set of video frames; wherein the spinal mobility features include the first set of spinal mobility features and the second set of spinal mobility features. In some embodiments, the first set of spinal mobility features correspond to a distance between a mid-back of the subject and a midpoint between a head and a tail of the subject, and wherein the second set of spinal mobility features correspond to an angle formed between the head, the tail and the mid-back of the subject. In some embodiments, the at least one memory includes further instructions, that when executed by the at least one processor, cause the system to: determine, using the video data, gait measurements of the subject for the duration of the video; and process, using the at least one machine learning model, the gait features to determine the visual frailty score for the subject. In some embodiments, the at least one memory includes further instructions, that when executed by the at least one processor, cause the system to: process the video data to determine point data tracking movement, for the duration of the video, of a set of body parts of the subject; determine, using the point data, a plurality of stance phases and a plurality of swing phases represented in the video data; determine, based on the plurality of stance phases and the plurality of swing phases, a plurality of stride intervals represented in the video data; and determine, using the point data, the gait measurements based on each stride interval of the plurality of stride intervals. In some embodiments, the at least one memory includes further instructions, that when executed by the at least one processor, cause the system to: determine a first transition from a first stance phase of the plurality of stance phases and a first swing phase of the plurality of swing phases based on a toe-off event of a left hind paw of the subject or a right hind paw of the subject; determine a second transition from a second swing phase of the plurality of swing phases to a second stance phase of the plurality of stance phases based on a foot strike event of the left hind paw or the right hind paw; and determine the gait measurements using the first transition and the second transition. In some embodiments, the at least one memory includes further instructions, that when executed by the at least one processor, cause the system to: process the video data to determine point data tracking movement, for the duration of the video, of a set of body parts of the subject, wherein the set of body parts comprises a left hind paw and a right hind paw, and wherein determining the gait measurements comprises: determine, using the point data, a step length for a stride interval, the step length representing a distance that the right hind paw travels past a previous left hind paw strike; determine, using the point data, a stride length using for the stride interval, the stride length representing a distance that the left hind paw travels during the each stride interval; determine, using the point data, a step width for the stride interval, the step width representing a distance between the left hind paw and the right hind paw. In some embodiments, the at least one memory includes further instructions, that when executed by the at least one processor, cause the system to: process the video data to determine point data tracking movement, for the duration of the video, of a set of body parts of the subject, wherein the set of body parts comprises a tail base, and wherein the instructions that cause the system to determine the gait measurements further cause the system to determine, using the point data, speed data of the subject based on movement of the tail base for a stride interval. In some embodiments, the at least one memory includes further instructions, that when executed by the at least one processor, cause the system to: process the video data to determine point data tracking movement, for the duration of the video, of a set of body parts of the subject, wherein the set of body parts comprises a tail base, and wherein the instructions that cause the system to determine the gait measurements further cause the system to: determine, using the point data, a set of speed data of the subject based on movement of the tail base during a set of frames representing a stride interval of the plurality of stride intervals; and determine a stride speed, for the stride interval, by averaging the set of speed data. In some embodiments, the at least one memory includes further instructions, that when executed by the at least one processor, cause the system to: process the video data to determine point data tracking movement, for the duration of the video, of a set of body parts of the subject, wherein the set of body parts comprises a right hind paw and a left hind paw, and wherein the instructions that cause the system to determine the gait measurements further cause the system to: determine, using the point data, first stance duration representing an amount of time that the right hind paw is in contact with ground during a stride interval; determine a first duty factor based on the first stance duration and the duration of the stride interval; determine, using the point data, second stance duration representing an amount of time that the left hind paw is in contact with ground during the stride interval; determine a second duty factor based on the second stance duration and the duration of the stride interval; and determine an average duty factor for the stride interval based on the first duty factor and the second duty factor. In some embodiments, the at least one memory includes further instructions, that when executed by the at least one processor, cause the system to: process the video data to determine point data tracking movement, for the duration of the video, of a set of body parts of the subject, wherein the set of body parts comprises a tail base and a neck base, and wherein the instructions that cause the system to determine the gait measurements further cause the system to: determine, using the point data, a set of vectors connecting the tail base and the neck base during a set of frames representing a stride interval of the plurality of stride intervals; and determine, using the set of vectors, an angular velocity of the subject for the stride interval. In some embodiments, the at least one memory includes further instructions, that when executed by the at least one processor, cause the system to: process the video data to determine point data tracking movement, for the duration of the video, of a set of body parts of the subject, wherein the set of body parts comprises a spine center of the subject, wherein a stride interval is associated with a set of frames of the video data, and wherein the instructions that cause the system to determine the gait measurements further cause the system to determine, using the point data, a displacement vector for the stride interval, the displacement vector connecting the spine center represented in a first frame of the set of frames and the spine center represented in a last frame of the set of frames. In some embodiments, the set of body parts also includes a nose of the subject, and wherein the instructions that cause the system to determine the metrics data further cause the system to: determine, using the point data, a set of lateral displacements of the nose for the stride interval based on a perpendicular distance of the nose from the displacement vector for each frame in the set of frames. In some embodiments, the lateral displacement of the nose is further based on a body length of the subject. In some embodiments, the instructions that cause the system to determine the gait measurements further cause the system to determine a tail tip displacement phase offset by: performing an interpolation using the set of lateral displacements of the nose to generate a smooth curve lateral displacement of the nose for the stride interval; determining, using the smooth curve lateral displacement of the nose, when a maximum displacement of the nose occurs during the stride interval; and determining a percent stride location representing a percent of the stride interval that is completed when the maximum displacement of the nose occurs. In some embodiments, the at least one memory includes further instructions, that when executed by the at least one processor, cause the system to: process the video data to determine point data tracking movement, for the duration of the video, of a set of body parts of the subject, wherein the set of body parts further comprises a tail base of the subject, and wherein the instructions that cause the system to determine the gait measurements further cause the system to determine, using the point data, a set of lateral displacements of the tail base for the stride interval based on a perpendicular distance of the tail base from the displacement vector for each frame in the set of frames. In some embodiments, the instructions that cause the system to determine the gait measurements further cause the system to determine a tail base displacement phase offset by: performing an interpolation using the set of lateral displacements of the tail base to generate a smooth curve lateral displacement of the tail base for the stride interval; determining, using the smooth curve lateral displacement of the tail base, when a maximum displacement of the tail base occurs during the stride interval; and determining a percent stride location representing a percent of the stride interval that is completed when the maximum displacement of the tail base occurs. In some embodiments, the at least one memory includes further instructions, that when executed by the at least one processor, cause the system to: process the video data to determine point data tracking movement, for the duration of the video, of a set of body parts of the subject, wherein the set of body parts comprises a tail tip of the subject, and wherein the instructions that cause the system to determine the gait measurements further cause the system to: determine, using the point data, a set of lateral displacements of the tail tip for the stride interval based on a perpendicular distance of the tail tip from the displacement vector for each frame in the set of frames. In some embodiments, the instructions that cause the system to determine the gait measurements further cause the system to determine a tail tip displacement phase offset by: performing an interpolation using the set of lateral displacements of the tail tip to generate a smooth curve lateral displacement of the tail tip for the stride interval; determining, using the smooth curve lateral displacement of the tail tip, when a maximum displacement of the tail tip occurs during the stride interval; and determining a percent stride location representing a percent of the stride interval that is completed when the maximum displacement of the tail tip occurs. In some embodiments, the at least one memory includes further instructions, that when executed by the at least one processor, cause the system to: process the video data to determine point data tracking movement, for the duration of the video, of a set of body parts, wherein the set of body parts comprises one or more of: the nose, base of neck, mid spine, left hind paw, right hind paw, base of tail, middle of tail and tip of tail; determine, using the point data, features for the subject; and process, using at least the one machine learning model, the features to determine the visual frailty score. In some embodiments, the at least one memory includes further instructions, that when executed by the at least one processor, cause the system to: process the video data using an additional machine learning model to identify a likelihood of the subject exhibiting a grooming behavior for a plurality of video frames of the video data; and determine the visual frailty score using the likelihood of the subject exhibiting the grooming behavior. In some embodiments, the at least one memory includes further instructions, that when executed by the at least one processor, cause the system to: process the video data using an additional machine learning model to identify a likelihood of the subject exhibiting a predetermined behavior for a plurality of video frames of the video data; and determine the visual frailty score using the likelihood of the subject exhibiting the predetermined behavior. In some embodiments, the at least one memory includes further instructions, that when executed by the at least one processor, cause the system to: determine a rotated set of video frames by rotating a first set of video frames of the video data; process the first set of video frames using a first machine learning model configured to identify a likelihood of the subject exhibiting a predetermined behavioral action; based on the processing of the first set of video frames by the first machine learning model, determine a first probability of the subject exhibiting the predetermined behavioral action in a first video frame of the first set of video frames, the first video frame corresponding to a first duration of the video data; process the rotated set of frames using the first machine learning model; based on the processing of the rotated set of video frames by the first machine learning model, determine a second probability of the subject exhibiting the predetermined behavioral action in a second video frame of the rotated set of video frames, the second video frame corresponding to the first duration of the video data; and use the first probability and the second probability, identifying a first label for the first video frame, the first label indicating that the subject exhibits the predetermined behavioral action. In some embodiments, the at least one memory includes further instructions, that when executed by the at least one processor, cause the system to: process the first set of video frames using a second machine learning model configured to identify a likelihood of the subject exhibiting the predetermined behavioral action; based on the processing of the first set of video frames by the second machine learning model, determine a third probability of the subject exhibiting the predetermined behavioral action in the first video frame; process the rotated set of video frames using the second machine learning model; based on the processing of the rotated set of video frames by the second machine learning model, determine a fourth probability of the subject exhibiting the predetermined behavioral action in the second video frame; and identify the first label using the first probability, the second probability, the third probability and the fourth probability. In some embodiments, the at least one memory includes further instructions, that when executed by the at least one processor, cause the system to: determine a reflected set of video frames by reflecting the first set of video frames; process the reflected set of video frames using the first machine learning model; based on the processing of the reflected set of video frames by the first machine learning model, determine a third probability of the subject exhibiting the predetermined behavioral action in a third video frame of the reflected set of frames, the third video frame corresponding to the first duration of the first video frame; and identify the first label using the first probability, the second probability, and the third probability. In some embodiments, the subject is a mouse, and the predetermined behavior comprises a grooming behavior comprising at least one of: paw licking, unilateral face wash, bilateral face wash, and flank licking. In some embodiments, first set of video frames represent a portion of the video data during a time period, and the first video frame is a last temporal frame of the time period. In some embodiments, the at least one memory includes further instructions, that when executed by the at least one processor, cause the system to: identify a second set of video frames from the video data; determine a second rotated set of video frames by rotating the second set of video frames; process the second set of video frames using the first machine learning model; based on the processing of the second set of video frames by the first machine learning model, determine a third probability of the subject exhibiting the predetermined behavioral action in a third video frame of the second set of video frames; process the second rotated set of video frames using the first machine learning model; based on the processing of the second rotated set of video frames by the first machine learning model, determine a fourth probability of the subject exhibiting the predetermined behavioral action in a fourth video frame of the rotated set of frames, the fourth video frame corresponding to the third video frame; and use the third probability and the fourth probability, identifying a second label for the fourth video frame, the second label indicating that the subject exhibits the predetermined behavioral action. In some embodiments, the first machine learning model is a machine learning classifier. In some embodiments, the at least one memory includes further instructions, that when executed by the at least one processor, cause the system to: process the video data to determine gait measurements for the subject for the duration of the video; process the video data to determine behavior data identifying portions of the video where the subject exhibits a predetermined behavior; and process, using the at least one machine learning model, the spinal mobility features, the gait measurements and the behavior data to determine the visual frailty score. In some embodiments, the video captures movements of the subject in an open field arena. In some embodiments, the at least one memory includes further instructions, that when executed by the at least one processor, cause the system to: determine a physical condition of the subject using the visual frailty score. In some embodiments, the physical condition is frailty. In some embodiments, the physical condition is a pre-frailty condition. In some embodiments, the subject is a mammal, optionally a mouse.

### BRIEF DESCRIPTION OF THE DRAWINGS

For a more complete understanding of the present disclosure, reference is now made to the following description taken in conjunction with the accompanying drawings.

FIG. 1 is a conceptual diagram of a system for determining a visual frailty score for a subject, according to embodiments of the present disclosure.

FIG. 2 is a flowchart illustrating a process for determining various data for the subject using point data derived from the video data, according to embodiments of the present disclosure.

FIG. 3 is a flowchart illustrating a process for determining morphometric data for the subject using ellipse data derived from the video data, according to embodiments of the present disclosure.

FIG. 4 is a flowchart illustrating a process for determining behavior data for the subject using the video data, according to embodiments of the present disclosure.

FIG. 5 is a flowchart illustrating a process for determining a visual frailty score using one or more the data determined according to the processes of FIGS. 2-4, according to embodiments of the present disclosure.

FIG. 6 is a block diagram conceptually illustrating example components of a device according to embodiments of the present disclosure.

FIG. 7 is a block diagram conceptually illustrating example components of a server according to embodiments of the present disclosure.

FIG. 8A-C presents a schematic drawing illustrating an automated visual frailty index (vFI) pipeline and graphs illustrating scores from manual frailty indexing of mice. The schematic of FIG. 8A illustrates a pipeline for creating an automated visual frailty index (vFI). Top-down videos of the open field for each mouse were processed by a tracking and segmentation network and a pose estimation network. The resulting frame-by-frame ellipse-fits and 12 point pose coordinates were further processed to produce per-video metrics for the mouse. Each mouse was also manually frailty indexed to produce an FI score. The video features for each mouse were used to model its FI score. FIG. 8B shows a scatter plot of FI score by age. The black line shows a piece-wise linear fit to the data; error bars show standard deviations. Males, lighter dots; females, darker dots. FIG. 8C shows a scatter plot of FI score by age from each scorer (Scorer 1, darkest dots; Scorer 2, lighter dots; Scorer 3, paler dots; Scorer 4, palest dots).

FIG. 9A-B presents graphs illustrating correlation between video metrics. A tight wrap of points around the diagonal line indicates a high correlation between mean and median or IQR and standard deviation for the respective metric. The graphs of FIG. 9A show correlation between average/mean (x-axis) and median (y-axis) video gait metrics. The diagonal line corresponds to maximum correlation (i.e., 1). The graphs of FIG. 9B show correlation between inter-quartile range (IQR, x-axis) and standard deviation (Stdev, y-axis) video gait metrics. The diagonal line corresponds to maximum correlation (i.e., 1).

FIG. 10A-I presents videographic images, schematic drawings, and graphs illustrating aspects of morphometric and behavioral measurements taken for visual frailty index scoring. FIG. 10A-I shows sample features used in the vFI. FIG. TOA shows a single frame of the top-down open-field video. FIG. 10B provides morphopemetric features from ellipse-fit and rear-paw distance measure performed on the mouse frame by frame. The major and minor axis of the ellipse fit are taken as the length and width respectively. FIG. 10C shows that the median ellipse-fit width and the median rear-paw distance taken over all mouse frames are highly correlated with FI score. FIG. 10D shows spatial, temporal, and whole-body coordination characteristics of gait used to create metrics [Sheppard, K. et al. Cell Reports 38, 110231 (January 2022)]. FIG. TOE shows the median step width and the inter-quartile range of tip-tail lateral displacement taken over all strides for a mouse are highly correlated with FI score. FIG. 10F shows spinal mobility measurements taken at each frame. dAC is the distance between point A and C (base of head and base of tail respectively) normalized for body length, dB is the distance of point B (mid-back) from the midpoint of the line AC, and aABC is the angle formed by the points A, B, and C. When the mouse spine is straight, dAC and aABC are at their maximum value while dB is at its minimum. When the mouse spine is bent, dB is at its maximum value while dAC and aABC are at their minimum. FIG. 10G shows the median of dB taken over all mouse frames and the median dB taken only over frames where the mouse is not in gait shows correlation with FI score. FIG. 10H shows a wall rearing event. The contour of the walls of the open field are taken and a buffer of 5 pixels is added (edge line), marking a threshold. The nose point of the mouse is tracked at each frame. A wall rearing event is defined by the nose point fully crossing the wall threshold. FIG. 10I shows the total count of rearing events and the number of rears in the first 5 minutes of the open-field video show some correlation with FI score.

FIG. 11A-D presents graphs illustrating certain analyses of FI scores by age and sex, and graphs illustrating comparison of male and female FI metrics. FIG. 11A provides the distribution of FI scores for males and females when the data are split into 4 age groups of equal range. The x-ticks represent the midpoint of each age group range. Significant difference in the distribution of male and female scores for that age group determined by the Mann-Whitney U test. FIG. 11B shows Pearson correlations of FI items with age for males compared to females. FIG. 11C shows Pearson correlations of video metrics with FI score for males compared to females. FIG. 11D shows Pearson correlations of video metrics with age for males compared to females. The oOpen-field, Gait, and Engineered key applies to FIGS. 11C and D.

FIG. 12A-G provides embodiments of prediction of age and frailty from video features. FIG. 12A provides a graphical illustration showing the different models that have been fit. FIG. 12B shows video features are more accurate in predicting age than clinical frailty index items. The performance of random forest models were compared using frailty parameters (FRIGHT) and video-generated features (vFRIGHT) in predicting age. FIG. 12C shows performance of ordinal regression models (classifiers) in terms of accuracy (accurately predicting the value of the frailty parameter in the test using the model trained on the training data). The black dotted line superimposed on the plot shows the accuracy that one would obtain if one guessed the values instead of using the video features. It was determined that the video features encode useful information that improves the models' ability to predict frailty parameter values accurately. FIG. 12D shows comparison among four models (LR*, SVM, RF, XGB) that predict FI score from video features in terms of the mean absolute error (MAE) and R2 shows that RF outperforms other models. FIG. 12E shows uncertainty in predicting age (column 1) and FI score (column 3) plotted as a function of age (weeks). The black curve shows the loess fit. These plots show less uncertainty in predicting age and FI scores for very young mice. The distributions of prediction interval (PI) widths were plotted and it was found that the PI widths for predicting Age are wider (increased uncertainty in predictions) for mice belonging to the middle age group (M). Similarly, the PI widths for predicting FI scores increase with Age in the data. FIG. 12F shows the residuals versus the index and predicted FI score versus true for training (columns 1 and 2) and test sets (columns 3 and 4) for the RF model. FIG. 12G shows the residuals versus the index and predicted age versus true for training (Columns 1 and 2) and test sets (Columns 3 and 4) for the RF model.

FIG. 13A-E provides quantile regression modeling of vFI using Generalized Random Forests. FIG. 13A shows variable importance measures for three quantile random forest models (lower tail—Q.025, median—Q.50, upper tail—Q.975). Mice in lower and upper tail correspond to mice with low and high frailty scores respectively. FIG. 13B provides marginal ALE plots showing how important features influence the predictions of the models on average. For example, the average predicted FI score rises with increasing step width, but falls for values greater than 3 in mice belonging to lower and upper tail. FIG. 13C provides a plot showing how strongly features interact with each other. FIG. 13D-E provide ALE second-order interaction plots for step width and step length1 (FIG. 13E: Width and Length) on the predicted FI score. Lighter shade indicates an above average and darker shade a below average prediction when the marginal effects from features are already taken into account. The FIG. 13D plot (resp. E) reveals a weak (resp. strong) interaction between step width and step length1 (resp. width and length). Large step width and step length1 increases the vFI score.

FIG. 14 provides a listing showing Feature Correlation with FI score.

FIG. 15 provides a listing showing vFI feature correlation (Pearson) with age.

FIG. 16 provides a listing showing Manual FI item correlation with age.

FIG. 17 provides an FI testing sheet listing all items for manual frailty indexing. Text in lighter font is modification from Whitehead, J. C. et al. Journals of Gerontology Series A: Biomedical Sciences and Medical Sciences 69, 621-632 (2014).

FIG. 18A-B provides graphs illustrating estimation of the scorer effect in clinical FI items. FIG. 18A illustrates the effect of tester varies across FI items. FIG. 18B shows the estimated random effect across 4 scorers in the data set.

FIG. 19A-I provides graphs and plots of detailed modeling analysis. FIG. 19A shows the distribution of age across 643 data points (533 mice). The distribution of manual Fladj scores across 643 data points (533 mice). FIG. 19B shows results relating to determination of the contributions of frailty parameters in predicting Age. The feature importance of all frailty parameters were calculated and it was determined that gait disorders, kyphosis, and piloerection have the highest contributions. FIG. 19C shows results indicating the random forest regression model performed better than other models with the lowest root-mean-squared error (RMSE) (p<2.2e−16, F3,147=59.53), and highest R2 (p<2.2e−16, F3,147=58.14) when compared using repeated-measures ANOVA. FIG. 19D shows the vFRIGHT model performed better than the FRIGHT model with a lower RMSE (RMSEvFRIGHT=17.97±1.44, RMSEFRIGHT=20.62±4.78, p<6.1e−7, F1,49=32.84) and higher R2 (RMSEvFRIGHT=0.78±0.04, RMSEFRIGHT=0.76±0.07, p<2.1e−8, F1,49=44.54) when compared using repeated-measures ANOVA. FIG. 19E shows the random forest regression model for predicting FI score on unseen future data performed better than all other models, with a lowest root-mean-squared error (RMSE) (p<8.3e−14, F3,147=26.62), and highest R2 (p<4.7e−14, F3,147=27.2). FIG. 19F plot shows the counts distribution (in each set of three values, 0—first of three, 0.5—second of three, 1—third of three) for individual frailty parameters—for many parameters such as Nasal discharge, Rectal prolapse, Vaginal uterine, and Diarrhea, the proportion of 0 counts is 1 (p0=1). Similarly, Dermatitis, Cataracts, Eye discharge swelling, Microphthalmia, Corneal opacity, Tail stiffening, and Malocclusions have p0>0.95. FIG. 19G shows the residuals versus the index and predicted versus true for training (Rows 1 and 2) and test sets (Rows 3 and 4) for the model that predicts Age using frailty index items for both training and test data. FIGS. 19H & I shows results of out-of-bag (OOB) error based 95% prediction intervals (PIs) (gray lines) quantifying uncertainty in point estimates/predictions (gray dots). There is one interval per test mouse, and approximately 95% of the PI intervals contain the correct Age (FIG. 19I) and FI scores (FIG. 19H). The x-axis (Test set index) was ordered in ascending order (from left to right) of the actual age/FI.

FIG. 20 shows results of Test for Simpson's paradox. Simpson [35] showed that the statistical relationship observed in the population could be reversed within all of the subgroups that make up that population, leading to erroneous conclusions drawn from the population data. To test for the manifestation of Simpson's paradox in the data, the bimodal Age distribution was split into two separate unimodal distributions (clusters), i.e., less than 70 weeks old (L70) versus more than 70 weeks old (U70). Next, the dependent variable (frailty) was plotted against each of the independent variables/features in the data and fit a simple linear regression model to each subgroup separately (data not shown) as well as to the aggregate data (data not shown). The correlations were quantified by measuring the slope of the linear fits of the features (Y) on Age (X). The slopes for L70, U70, and overall (All) were computed and the slopes were plotted for features in decreasing order of their relevance to the model (where Age was predicted from these features). For each set of three bars, the left bar is L70, middle bar is U70, and right bar is All. It was determined that Simpson's paradox does not manifest in any of the top fifteen features in the data.

FIG. 21A-E provides graphs from further experiments to test model performance and parameters. FIG. 20A shows results of comparison of the performance of different feature sets, 1) age alone, 2) video, and 3) age+video, in predicting frailty. Age alone was used as a feature in a linear (AgeL) and a generalized additive non-linear model (AgeG). Although a clear improvement of the random forest model (VideoRF) using video features was not observed over a vFI prediction based on age alone, a clear improvement in prediction performance was seen for the model (AllRF), which contains video features+age with lowest MSE (p<2.2e−16, F3,147=213.79, LMM post hoc pairwise comparison with AgeG, t147=−12.21, FDR-adjusted p<0.0001), lowest RMSE (p<2.2e−16, F3,147=172.88, LMM post hoc pairwise comparison with AgeG, t147=−14.12, FDR-adjusted p<0.0001) and highest R2 (p<2.2e−16, F3,147=171.12, LMM post hoc pairwise comparison with AgeG, t147=14.07, FDR-adjusted p<0.0001). This shows that video features add important information pertaining to frailty that age alone does not. FIG. 21B shows results of embodiment with selected animals whose ages and FI scores had an inverse relationship, i.e., younger animals with higher FI scores and older animals with lower FI scores. Five (5) test sets containing animals with these criteria were formed and the random forest (RF) model was trained on the remaining mice. The model using only video features (VideoRF) does better than all other models for these mice with lowest MSE (p<1.6e−08, F3,12=91.07, LMM post hoc pairwise comparison with AgeG, t12=13.60, FDR-adjusted p<0.0001), lowest RMSE (p<1.6e−08, F3,12=93.88, LMM post hoc pairwise comparison with AgeG, t12=14.15, FDR-adjusted p<0.0001) and highest R2 (p<1.31e−08, F3,12=94.32, LMM post hoc pairwise comparison with AgeG, t12=14.10, FDR-adjusted p<0.0001). FIG. 21C shows results of further investigation of the difference between Age and vFI predictors in terms of feature importance. Features lying along the diagonal are important for both Age and vFI predictions. FIG. 21D shows results of predicting FI score from video features extracted from videos of shorter durations. Video features generated from videos with shorter durations (first 5 and 20 minutes) were used to investigate the loss in accuracy in predicting age and FI score. The random forest model trained with features generated from 60-minute videos was used as a baseline model for comparison. A diminished loss in accuracy was found using shorter videos. FIG. 21E shows results of study to see how much training data is realistically needed. A simulation study was performed where a different percentage of total data was allocated to training. As expected, there is a general downward (upward) trend in MAE, RMSE (R2 with an increasing percentage of data allocated to training set. Indeed, a smaller training set (<80% training) can reach a similar training performance.

### DETAILED DESCRIPTION

Chronological aging is uniform, but biological aging is heterogeneous. Clinically, this heterogeneity manifests itself in health status and mortality, and distinguishes healthy from unhealthy aging. Clinical frailty indexes serve as an important tool in gerontology to capture health status. Frailty indexes have been adapted for use in mice and are an effective predictor of mortality risk. To accelerate understanding of biological aging, high-throughput approaches to pre-clinical studies are necessary. Currently, however, mouse frailty indexing is manual and relies on trained/expert manual scorers, which imposes limits on scalability and reliability in generating the frailty index.

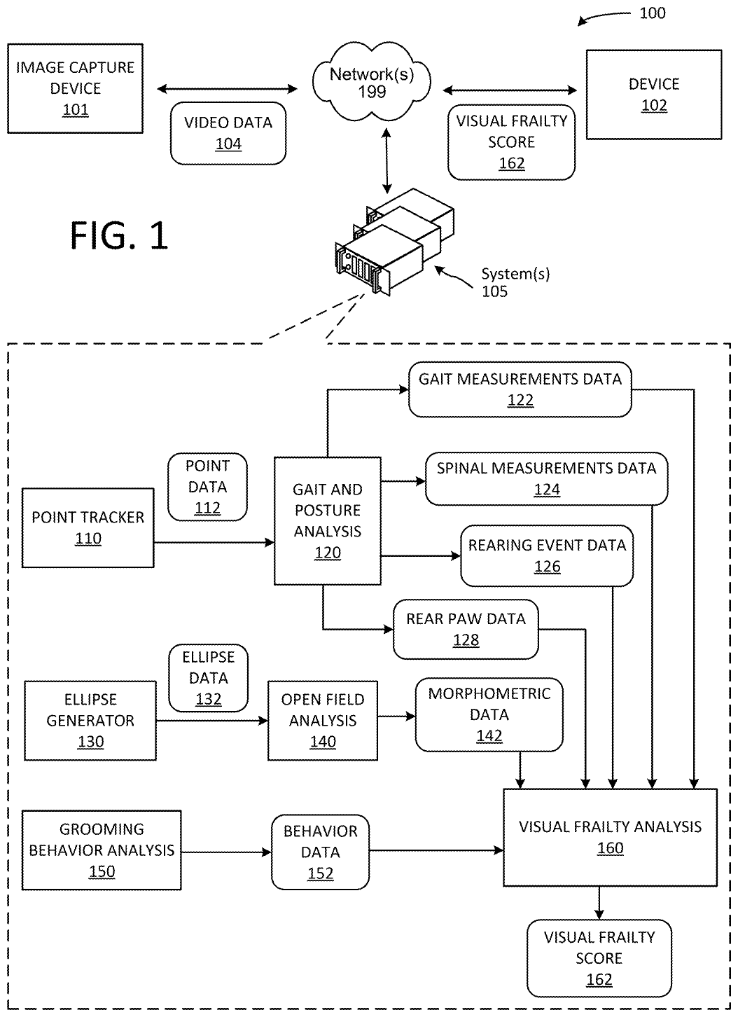

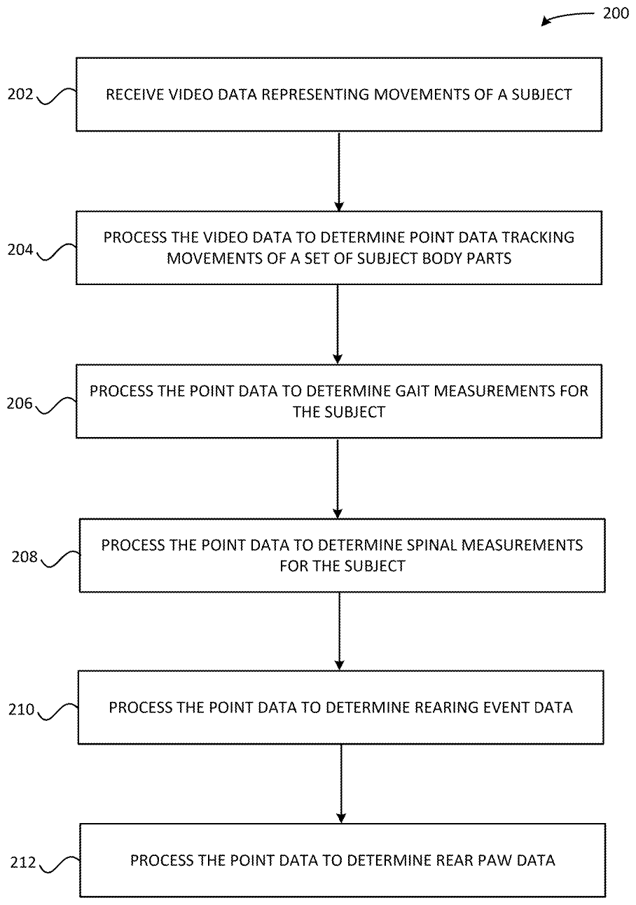

The present disclosure relates to an automated visual frailty system that processes video data of a subject and generates a visual frailty score for the subject. The automated visual frailty system (e.g., system 100 shown in FIG. 1) of the present disclosure may use one or more machine learning based techniques to determine the visual frailty score for a subject, and may operate on video data from an open field assay. The automated visual frailty system may determine the visual frailty score for the subject based on features of biological aging extracted from the video data. In some embodiments, the automated visual frailty system may extract morphometric features, gait and posture features, behavioral features and other features from the video data that may be used to determine the visual frailty score. The automated visual frailty system may result in increased accuracy, reproducibility, scalability, and efficiency in generating frailty indexes for subjects.

The system 100 of the present disclosure may operate using various components as illustrated in FIG. 1. The system 100 may include an image capture device 101, a device 102 and one or more systems 105 connected across one or more networks 199. The image capture device 101 may be part of, included in, or connected to another device (e.g., device 600), and may be a camera, a high speed video camera, or other types of devices capable of capturing images and videos. The device 101, in addition to or instead of an image capture device, may include a motion detection sensor, infrared sensor, temperature sensor, atmospheric conditions detection sensor, and other sensors configured to detect various characteristics/environmental conditions. The device 102 may be a laptop, a desktop, a tablet, a smartphone, or other types of computing devices capable of displaying data, and may include one or more components described in connection with device 600 below.

The image capture device 101 may capture video (or one or more images) of a subject, and may send video data 104 representing the video to the system(s) 105 for processing as described herein. The video may include movements of the subject in an open field arena. In some cases, the video data 104 may correspond to images (image data) captured by the device 101 at certain time intervals, such that the images captures movements of the subject over a period of time. The system(s) 105 may include one or more components shown in FIG. 1, and may be configured to process the video data 104 to determine a visual frailty score for the subject. The system(s) 105 may generate a visual frailty score 162 corresponding to the subject. The system(s) 105 may send the visual frailty score 162 to the device 102 for output to a user to observe the results of processing the video data 104.

In some embodiments, the video data 104 may include video of more than one subject, and the system(s) 105 may process the video data 104 to determine features and visual frailty scores for each subject represented in the video data 104.

The system(s) 105 may be configured to determine various features from the video data 104 for the subject. For determining these features and for determining the visual frailty score, the system(s) 105 may include multiple different components. As shown in FIG. 1, the system(s) 105 may include a point tracker component 110, a gait and posture analysis component 120, an ellipse generator component 130, an open field analysis component 140, a grooming behavior analysis component 150, and a visual frailty analysis component 160. The system(s) 105 may include fewer or more components than shown in FIG. 1. These various components, in some embodiments, may located on the same physical system 105. In other embodiments, one or more of the various components may be located on different/separate physical systems 105. Communication between the various components may occur directly or may occur across a network(s) 199. Communication between the device 101, the system(s) 105 and the device 102 may occur directly or across a network(s) 199.

In some embodiments, one or more components shown as part of the system(s) 105 may be located at the device 102 or at a computing device (e.g., device 600) connected to the image capture device 102.

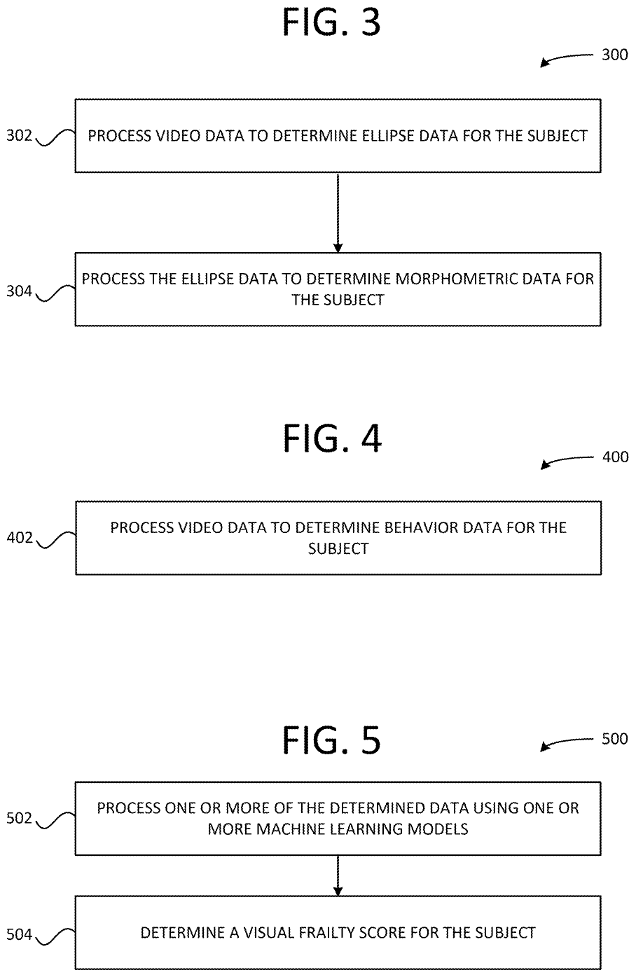

At a high-level, the system(s) 105 may be configured to process the video data 104 to determine point data (which may be referred to as pose estimation data in the examples below). Using the point data, the system(s) 105 may determine various features corresponding to the subject's movements in the video, such as, gait measurements, spinal measurements, rearing events, rear paw measurements, etc. Details on determining the point data and the various features from the point data are described below in relation to FIG. 2. The system(s) 105 may also be configured to determine ellipse data (which may be referred to as ellipse-fit in the examples below). Using the ellipse data, the system(s) 105 may determine morphometric data for the subject. Details on determining the ellipse data and the morphometric data are described below in relation to FIG. 3. The system(s) 105 may also be configured to determine behavior features of the subject using the video data 104. Details on determining the behavior features are described below in relation to FIG. 4. Using the determined features/data, the system(s) 105 may then determine the visual frailty score 162 for the subject. Details on determining the visual frailty score 162 are described below in relation to FIG. 5.

FIG. 2 is a flowchart illustrating a process 200 for determining various data for the subject using point data derived from the video data, according to embodiments of the present disclosure. One or more of the steps 200 may be performed in another order/sequence than shown in FIG. 2. One or more steps of the process 200 may be performed by the point tracker component 110 and/or the gait and posture analysis component 120.

At a step 202, the point tracker component 110 may receive the video data 104 representing movements of the subject. At a step 204, the point tracker component 110 may process the video data 104 to determine point data 112 tracking movements of a set of subject body parts. The point tracker component 110 may be configured to identify various body parts of the subject. These body parts may be identified using various point data, such that first point data may correspond to a first body part, second point data may correspond to a second body part, and so on. The point data may be, in some embodiments, one or more pixel locations/coordinates (x,y) corresponding to the body part. As such, the point data 112 may include multiple point data corresponding to multiple body parts. The point tracker component 110 may be configured to identify pixel locations corresponding to a particular body part within one or more video frames of the video data 104. The point tracker component 110 may track movement of the particular body part for the duration of the video by identifying the corresponding pixel locations during the video. The point data 112 may indicate the location of the particular body part during a particular frame of the video. The point data 112 may include the locations of all the body parts being identified and tracked by the point tracker component 110 over multiple frames of the video data 104. The point data 112 may also include a confidence score relating to the location of a particular body part in a particular video frame. The confidence score may indicate how confident the point tracker component 110 is in determining that particular location. The confidence score may be a probability/likelihood of the particular body part being at that particular location.

In some embodiments, where the subject is a mouse, the point tracker component 110 may identify and track the following body parts: nose, left ear, right ear, base of neck, left forepaw, right forepaw, mid spine, left rear paw, right rear paw, base of tail, mid tail and tip of tail.

The point data 112 may be a vector, an array, or a matrix representing pixel coordinates of the various body parts over multiple video frames. For example, the point data 112 may be [frame1={nose: (x1, y1); right rear paw: (x2, y2)}], [frame2={nose: (x3, y3); right rear paw: (x4, y4)}], etc. The point data 112, for each frame, may include in some embodiments at least 12 pixel coordinates representing 12 portions/body parts of the subject that the point tracker component 110 is configured to track.

The point tracker component 110 may implement one or more pose estimation techniques. The point tracker component 110 may include one or more machine learning models configured to process the video data 104. In some embodiments, the one or more machine learning models may be a neural network such as, a deep neural network, a deep convolutional neural network, a recurrent neural network, etc. In other embodiments, the one or more machine learning models may be other types of models than a neural network. The ML model(s) of the point tracker component 110 may be configured for 3D markerless pose estimation based on transfer learning with deep neural networks.

The point tracker component 110 may be configured to determine the point data 112 with high accuracy and precision because the visual frailty score 162 may be sensitive to errors in the point data 112. The point tracker component 110 may implement an architecture that maintains high-resolution features throughout the machine learning model stack, thereby preserving spatial precision. In some embodiments, the point tracker component 110 architecture may include one or more transpose convolutions to cause matching between a heatmap output resolution and the video data 104 resolution. The point tracker component 110 may be configured to determine the point data 112 in near real-time speeds and may run a high processing capacity GPU. The point tracker component 110 may be configured such that modifications and extensions can be made easily. In some embodiments, the point tracker component 110 may be configured to generate an inference at a fixed scale, rather than processing at multiple scales, to save computing resources and time.

In some embodiments, the video data 104 may track movements of one subject, and the point tracker component 110 may not be configured to perform any object detection techniques/algorithms to detect the subject within the video frame. In other embodiments, the video data 104 may track movements of more than one subject, and the point track component 110 may be configured to perform object detection techniques to identify one subject from another subject within the video data 104.

At a step 206, the gait and posture analysis component 120 may process the point data 112 to determine gait measurements data 122 for the subject. The gait and posture analysis component 120 may determine distances and/or angles between various subject body parts using the point data 112.

The gait and posture analysis component 120 may determine distances between various body parts of the subject(s) and generate one or more distance vectors. The gait and posture analysis component 120 may determine a first distance between two (a first pair) of body parts, a second distance between another two (a second pair) of body parts, and so on, for each video frame of the video data 104, and the first and second distances may be included in the distance vectors. In some embodiments, the gait and posture analysis component 120 may determine a first distance feature vector representing distances between a first pair of body parts for multiple video frames, a second distance feature vector representing distances between a second pair of body parts for multiple video frames, and so on. Each value in the first distance vector may represent a distance between the first pair of body parts for a different corresponding video frame of the video data 104. In some embodiments, the distance vectors may be included in the gait measurements data 122 to be used to determine the visual frailty score 162. In other embodiments, the distance vectors may be used by the gait and posture analysis component 120 to determine data to be included in the gait measurements data 122.

The gait and posture analysis component 120 may determine an angle between various body parts of the subject(s) and generate one or more angle vectors. The gait and posture analysis component 120 may determine first angle data between three (a first trio) of body parts, second angle data between another three (a second trio) of body parts, and so on, for multiple video frames. The gait and posture analysis component 120 may determine a first angle vector representing angles between a first trio of body parts over multiple video frames, a second angle vector representing angles between a second trio of body parts over multiple video frames, and so on. Each value in the first angle vector may represent an angle between the first trio of body parts for a different corresponding video frame of the video data 104. In some embodiments, the angle vectors may be included in the gait measurements data 122 to be used to determine the visual frailty score 162. In other embodiments, the angle vectors may be used by the gait and posture analysis component 120 to determine data to be included in the gait measurements data 122.

In some embodiments, the gait and posture analysis component 120 may determine gait and posture metrics. As used herein, gait metrics may refer to metrics derived from the subject's paw movements. Gait metrics may include, but is not limited to, step width, step length, stride length, speed, angular velocity, and limb duty factor. As used herein, posture metrics may refer to metrics derived from the movements of the subject's whole body. In some embodiments, the posture metrics may be based on movements of the subject's nose and tail. Posture metrics, may include, but is not limited to, lateral displacement of nose, lateral displacement of tail base, lateral displacement of tail tip, nose lateral displacement phase offset, tail base displacement phase offset, and tail tip displacement phase offset. One or more of the gait and posture metrics may be included in the gait measurements data 122. In some embodiments, each of the gait and posture metrics may be provided to the visual frailty analysis component 160 as separate inputs rather than a collective input via the gait measurements data 122.

The gait and posture analysis component 120 may determine one or more of the gait and posture metrics on a per-stride basis. The gait and posture analysis component 120 may determine a stride interval(s) represented in a video frame of the video data 104. In some embodiments, the stride interval may be based on a stance phase and a swing phase. In example embodiments, the approach for detecting stride intervals is based on the cyclic structure of gait. During a stride cycle, each of the paws may have a stance phase and a swing phase. During the stance phase, the subject's paw is supporting the weight of the subject and is in static contact with the ground. During the swing phase, the paw is moving forward and is not supporting the subject's weight. The transition from a stance phase to a swing phase is referred to herein as a toe-off event, and the transition from a swing phase to a stance phase is referred to herein as a foot-strike event.

The gait and posture analysis component 120 may determine a plurality of stance and swing phases represented in a duration of the video data 104. In an example embodiment, the stance and swing phases may be determined for the hind paws of the subject. The gait and posture analysis component 120 may calculate a paw speed and may infer that a paw is in the stance phase when the speed falls below a threshold value, and may infer that the paw is in the swing phase when it exceeds that threshold value. The gait and posture analysis component 120 may determine that the foot strike events occur in the video frame where the transition from the swing phase to the stance phase occurs.

The gait and posture analysis component 120 may also determine the stride intervals represented in the time period. A stride interval may span over multiple video frames of the video data 104. The gait and posture analysis component 120, for example, may determine that a time period of 10 seconds has 5 stride intervals, and that one of the 5 stride intervals is represented in 5 consecutive video frames of the video data 104. In an example embodiment, the left hind foot strike event may be defined as the event that separates/differentiates stride intervals. In another example embodiment, the right hind foot strike event may be defined as the event that separates/differentiates the stride intervals. In yet another example embodiment, a combination of the left hind foot strike event and the right hind foot strike event may be used to define the separate stride intervals. In some other embodiments, the gait and posture analysis component 120 may determine the stance and swing phases for the fore paws, may calculate a paw speed based on the fore paws, and may differentiate between the stride intervals based on the right and/or left forepaw foot strike event. In some other embodiments, the transition from the stance phase to the swing phase—the toe-off event—may be used to separate/differentiate the stride intervals.

In some embodiments, it may be preferred to determine the stride intervals based on a hind foot strike event, rather than a forepaw strike event due to the point data 112 inference quality (determined by the point tracker component 110) for the forepaws, in some cases, being of low confidence. This is may be a result of the forepaws being occluded more often than the hind paws from within a top-down view, and therefore the forepaws being more difficult to accurately locate.

The gait and posture analysis component 120 may filter the determined stride intervals to determine which stride intervals are used to determine the gait and posture metrics. In some embodiments, such filtering may remove spurious or low confidence stride intervals. In some embodiments, the criteria for removing the stride intervals may include, but is not limited to: low confidence point data estimate, physiologically unrealistic point data estimates, missing right hind paw strike event, and insufficient overall body speed of subject (e.g., a speed under 10 cm/sec). In some embodiments, the filtering of the stride intervals may be based on a confidence level in determining the point data 112 used to determine the stride intervals. For example, stride intervals determined with a confidence level below a threshold value may be removed from the set of stride intervals used to determine the gait and posture metrics. In some embodiments, the first and last strides are removed in a continuous sequence of strides to avoid starting and stopping behaviors from adding noise to the data to be analyzed. For example, a sequence of seven strides will result in at most five strides being used for analysis.

After determining the stride intervals represented in the video data 104, the gait and posture analysis component 120 may determine the gait and posture metrics to be included in the gait measurements data 122. The gait and posture analysis component 120 may determine, using the point data 112, a step length for each of the stride intervals. In some embodiments, the point data 112 may be for a left hind paw, a left forepaw, a right hind paw and a right forepaw of the subject. In some embodiments, the step length may be a distance between the left forepaw and the right hind paw for the stride interval. In some embodiments, the step length may be a distance between the right forepaw and the left hind paw for the stride interval. In some embodiments, the step length may be a distance that the right hind paw travels past the previous left hind paw strike.

The gait and posture analysis component 120 may determine, using the point data 112, a stride length for a stride interval. The gait and posture analysis component 120 may determine a stride length for each stride interval for the time period. In some embodiments, the point data 112 may be for a left hind paw, a left forepaw, a right hind paw and a right forepaw of the subject. In some embodiments, the stride length may be a distance between the left forepaw and the left hind paw for the each stride interval. In some embodiments, the stride length may be a distance between the right forepaw and the right hind paw. In some embodiments, the stride length may be the full distance that the left hind paw travels for a stride from a toe-off event to a foot-strike event.

The gait and posture analysis component 120 may determine, using the point data 112, a step width for a stride interval. The gait analysis component 120 may determine a step width for each stride interval for the time period. In some embodiments, the point data 112 may be for a left hind paw, a left forepaw, a right hind paw and a right forepaw for the subject. In some embodiments, the step width is a distance between the left fore paw and the right fore paw. In some embodiments, the step width is a distance between the left hind paw and the right hind paw. In some embodiments, the step width is an averaged lateral distance separating hind paws. This may be calculated as length of the shortest line segment that connects the right hind paw strike to the line that connects the left hind paw's toe-off location to its subsequent foot strike position.

The gait and posture analysis component 120 may determine, using the point data 112, a paw speech for a stride interval. The gait and posture analysis component 120 may determine a paw speed for each stride interval for the time period. In some embodiments, the point data 112 may be for a left hind paw, a right hind paw, a left forepaw, and a right forepaw for the subject. In some embodiments, the paw speed may be a speed of one of the paws during the stride interval. In some embodiments, the paw speed may be a speed of the subject and may be based on a tail base of the subject.

The gait and posture analysis component 120 may determine, using the point data 112, a stride speed for a stride interval. The gait and posture analysis component 120 may determine a stride speed for each stride interval for the time period. In some embodiments, the point data 112 may be for a tail base. In some embodiments, the stride speed may be determined by determining a set of speed data for the subject based on the movement of the subject tail base during a set of video frames representing the stride interval. Each speed data in the set of speed data may correspond to one frame of the set of video frames. The stride speed may be calculated by averaging (or combining in another manner) the set of speed data.

The gait and posture analysis component 120 may determine, using the point data 112, a limb duty factor for a stride interval. The gait and posture analysis component 120 may determine a limb duty factor for each stride interval for the time period. In some embodiments, the point data 112 may be for a right hind paw and a left hind paw of the subject. In some embodiments, the limb duty factor for the stride interval may be an average of a first duty factor and a second duty factor. The gait and posture analysis component 120 may determine a first stance time representing an amount of time that the right hind paw is in contact with the ground during the stride interval, and then may determine the first duty factor based on the first stance time and the length of time for the stride interval. The gait and posture analysis component 120 may determine a second stance time representing an amount of time that the left hind paw is in contact with the ground during the stride interval, and then may determine the second duty factor based on the second stance time and the length of time for the stride interval. In other embodiments, the limb duty factor may be based on the stance time and duty factors of the forepaws.

The gait and posture analysis component 120 may determine, using the point data 112, an angular velocity for a stride interval. The gait and posture analysis component 120 may determine an angular velocity for each stride interval for the time period. In some embodiments, the point data 112 may be for a tail base and a neck base of the subject. The gait and posture analysis component 120 may determine a set of vectors connecting the tail base and the neck base, where each vector in the set corresponds to a frame of a set of frames for the stride interval. The gait and posture analysis component 120 may determine the angular velocity based on the set of vectors. The vectors may represent an angle of the subject, and a first derivative of the angle value may be the angular velocity for the frame. In some embodiments, the gait and posture analysis component 120 may determine a stride angular velocity by averaging the angular velocities for the frames for the stride intervals.

The gait and posture analysis component 120 may determine lateral displacements of a nose, a tail tip and a tail base on the subject for individual stride intervals. Based on the lateral displacements of the nose, the tail tip, and the tail base, the gait and posture analysis component 120 may determine a displacement phase offset of each of the respective subject body part. To determine the lateral displacements, the gait and posture analysis component 120 may first determine, using the point data 112, a displacement vector for a stride interval. The gait and posture analysis component 120 may determine the displacement vector for each stride interval for the time period. In some embodiments, the point data 112 may be for a spine center of the subject. The stride interval may span over multiple video frames. In some embodiments, the displacement vector may be a vector connecting the spine center in a first video frame of the stride interval and the spine center in the last video frame of the stride interval.

The gait and posture analysis component 120 may determine, using the point data 112 and the displacement vector, a lateral displacement of the subject nose for the stride interval. The gait and posture analysis component 120 may determine the lateral displacement of the nose for each stride interval for the time period. In some embodiments, the point data 112 may be for a spine center and a nose of the subject. In some embodiments, the gait and posture analysis component 120 may determine a set of lateral displacements of the nose, where each lateral displacement of the nose may correspond to a video frame of the stride interval. The lateral displacement may be a perpendicular distance of the nose, in the respective video frame, from the displacement vector for the stride interval. In some embodiments, the gait and posture analysis component 120 may subtract the minimum distance from the maximum distance and divide that by the subject body length so that the displacement measured in larger subjects may be comparable to the displacement measured in smaller subjects.

The gait and posture analysis component 120 may determine, using the set of lateral displacements of the nose for the stride interval, a nose displacement phase offset. The gait and posture analysis component 120 may perform an interpolation using the set of lateral displacements of the nose to generate a smooth curve lateral displacement of the nose for the stride interval, then may determine, using the smooth curve lateral displacement of the nose, when a maximum displacement of the nose occurs during the stride interval. The gait and posture analysis component 120 may determine a percent stride location representing a percent of the stride interval that is completed when the maximum displacement of the nose occurs. In some embodiments, the gait and posture analysis component 120 may perform a cubic spline interpolation in order to generate the smooth curve for the displacement, and because of the cubic interpolation the maximum displacement may occur at time points between video frames.

The gait and posture analysis component 120 may determine, using the point data 112 and the displacement vector, a lateral displacement of the subject tail base for the stride interval. The gait and posture analysis component 120 may determine the lateral displacement of the tail base for each stride interval for the time period. In some embodiments, the point data 112 may be for a spine center and a tail base of the subject. In some embodiments, the gait and posture analysis component 120 may determine a set of lateral displacements of the tail base, where each lateral displacement of the tail base may correspond to a video frame of the stride interval. The lateral displacement may be a perpendicular distance of the tail base, in the respective video frame, from the displacement vector for the stride interval. In some embodiments, the gait and posture analysis component 120 may subtract the minimum distance from the maximum distance and divide that by the subject body length so that the displacement measured in larger subjects may be comparable to the displacement measured in smaller subjects.

The gait and posture analysis component 120 may determine, using the set of lateral displacements of the tail base for the stride interval, a tail base displacement phase offset. The gait and posture analysis component 120 may perform an interpolation using the set of lateral displacements of the tail base to generate a smooth curve lateral displacement of the tail base for the stride interval, then may determine, using the smooth curve lateral displacement of the tail base, when a maximum displacement of the nose occurs during the stride interval. The gait and posture analysis component 120 may determine a percent stride location representing a percent of the stride interval that is completed when the maximum displacement of the tail base occurs. In some embodiments, the gait and posture analysis component 120 may perform a cubic spline interpolation in order to generate the smooth curve for the displacement, and because of the cubic interpolation the maximum displacement may occur at time points between video frames.

The gait and posture analysis component 120 may determine, using the point data 112 and the displacement vector, a lateral displacement of the subject tail tip for the stride interval. The gait and posture analysis component 120 may determine the lateral displacement of the tail tip for each stride interval for the time period. In some embodiments, the point data 112 may be for a spine center and a tail tip of the subject. In some embodiments, the gait and posture analysis component 120 may determine a set of lateral displacements of the tail tip, where each lateral displacement of the tail tip may correspond to a video frame of the stride interval. The lateral displacement may be a perpendicular distance of the tail tip, in the respective video frame, from the displacement vector for the stride interval. In some embodiments, the gait and posture analysis component 120 may subtract the minimum distance from the maximum distance and divide that by the subject body length so that the displacement measured in larger subjects may be comparable to the displacement measured in smaller subjects.

The gait and posture analysis component 120 may determine, using the set of lateral displacements of the tail tip for the stride interval, a tail base displacement phase offset. The gait and posture analysis component 120 may perform an interpolation using the set of lateral displacements of the tail tip to generate a smooth curve lateral displacement of the tail tip for the stride interval, then may determine, using the smooth curve lateral displacement of the tail tip, when a maximum displacement of the nose occurs during the stride interval. The gait and posture analysis component 120 may determine a percent stride location representing a percent of the stride interval that is completed when the maximum displacement of the tail tip occurs. In some embodiments, the gait and posture analysis component 120 may perform a cubic spline interpolation in order to generate the smooth curve for the displacement, and because of the cubic interpolation the maximum displacement may occur at time points between video frames.

In some embodiments, the gait and posture analysis component 120 may include a statistical analysis component that may take as input the gait and posture metrics to perform some statistical analysis. Subject body size and subject speed can affect the gait and/or posture metrics of the subject. For example, a subject that moves faster may have a different gait than a subject that moves slow. As a further example, a subject with a larger body will have a different gait than a subject with a smaller body. However, in some cases a difference (as compared to a control subject) in stride speed may be a defining feature of gait and posture changes due to aging and frailty. The gait and posture analysis component 120 collects multiple repeated measurements for each subject (via the video data 104 and a subject in an open area), and each subject has a different number of strides giving rise to imbalanced data. Averaging over repeated strides, which yields one average value per subject, may be misleading as it removes variation and introduces false confidence. At the same time, classical linear models do not discriminate between stable intra-subject variations and inter-subject fluctuations, which can bias the statistical analysis. To address these issues, the gait and posture analysis component 120, in some embodiments, employ a linear mixed model(s) (LMM) to dissociate within-subject variation from genotype-based variation between subjects. In some embodiments, the gait and posture analysis component 120 may capture the main effects such as subject size, genotype, age, and may additionally capture a random effect for the intra-subject variation. The techniques of the invention collects multiple repeated measurements at different ages of the subject giving rise to a nested hierarchical data structure. Example statistical models implemented at the gait and posture analysis component 120 are shown below as models M1, M2 and M3. These models follow the standard LMM notation with (Genotype, BodyLength, Speed, TestAge) denoting the fixed effects and (SubjectID/TestAge) (where the test age is nested within the subject) denoting the random effect.

$$Phenotype \sim Genotype + TestAge + BodyLength + ( 1 \vert MouseID / TestAge ) \qquad M 1$$

$$Phenotype \sim Genotype + TestAge + Speed + ( 1 \vert MouseID / TestAge ) \qquad M2$$

$$Phenotype \sim Genotype + TestAge + Speed + BodyLength + ( 1 \vert MouseID / TestAge ) \qquad M 3$$

The model M1 take age and body length as inputs, the model M2 take age and speed as inputs, and the model M3 take age, speed and body length as inputs. In some embodiments, the models of the gait and posture analysis component 120 does not include subject sex as an effect because the sex may be highly correlated with the body length/size of the subject. In other embodiments, the models of the gait and posture analysis component 120 may take subject sex as an input. Using the point data 112 (determined by the point tracker component 110), enables determination of subject body size and speed for these models. Therefore, no additional measurements are needed to these variables for the models.

One or more of the data included in the gait measurements data 122 may be circular variables (e.g., stride length, angular velocities, etc.), and the gait and posture analysis component 120 may implement a function of linear variables using a circular-linear regression model. The linear variables, such as body length and speed, may be included as covariates in the model. In some embodiments, the gait and posture analysis component 120 may implement a multivariate outlier detection algorithm at the individual subject level to identify subjects with injuries and developmental effects.

In some embodiments, the gait measurements data 122 may include, for the subject, one or more of speed, velocity, angular velocity, step length, step width, stride length, lateral displacement, limb duty factor, temporal symmetry, stride count for the duration of the video, and distance covered. Table 1 provides a list of video features and metrics used in certain embodiments of the invention.

Table 1 lists video features.

| Category | Name | Description | Units |
|---|---|---|---|
| Open Field | distance_cm | Sum of locomotor | centimeters |
| | | activity | |
| Open Field | center_time_secs | Sum of time spent | seconds |
| | | in center | |
| Open Field | periphery_time_secs | Sum of time spent | seconds |
| | | along any wall | |
| Open Field | corner_time_secs | Sum of time spent | seconds |
| | | in any corner | |
| Open Field | center_distance_cm | Average distance | centimeters |
| | | from center | |
| | | across the video | |
| Open Field | periphery_distance_cm | Average distance from | centimeters |
| | | nearest periphery | |
| | | across the video | |
| Open Field | corner_distance_cm | Average distance | centimeters |
| | | from nearest corners | |
| | | acrossthe video | |
| Open Field | grooming_number_bouts | Sum of all grooming | ~ |
| | | bouts in video | |
| Open Field | grooming_duration_secs | Average length of | seconds |
| | | grooming bouts | |
| Gait | angular_velocity | The first derivative | degrees/second |
| | | of angle of a mouse, | |
| | | determined by the | |
| | | vector connecting the | |
| | | mouse's base of tail | |
| | | to its base of neck | |
| Gait | lateral_displacement | The difference between | ~ |
| | | the minimum and | |
| | | maximum values of | |
| | | a reference point's | |
| | | perpendicular | |
| | | distance from the | |
| | | mouse's displacement | |
| | | vector for a stride for | |
| | | each frame of a stride, | |
| | | normalized by the | |
| | | mouse's body length. | |
| | | The referece points | |
| | | used are nose, | |
| | | base of tail, | |
| | | and tail tip. | |
| Gait | limb_duty_factor | The amount of time that | ~ |
| | | the paw is in contactwith | |
| | | the ground divided by | |
| | | the full stride time, | |
| | | calculated and averaged | |
| | | for each hind paw | |
| Gait | speed_cm_per_sec | Speed is determined by | Centimeters |
| | | the base of tail point | per second |
| Gait | step_length | The distance that the | centimeters |
| | | right hind paw travels | |
| | | past the previous opposite | |
| | | paw strike. Step_length 1 | |
| | | uses left hind paw | |
| | | strike, whilestep_length2 | |
| | | uses right hind paw strike | |
| Gait | step_width | The length of the shortest | centimeters |
| | | line segment that | |
| | | connects the right hind | |
| | | paw strike to the line | |
| | | that connects the left | |
| | | hind paw's toe-off | |
| | | location to its | |
| | | subsequent foot strike | |
| | | position. | |
| Gait | stride_length | The full distance that | centimeters |
| | | the left hind paw travels | |
| | | for a stride, from | |
| | | toe-off to foot-strike | |
| Gait | temporal_symmetry | The difference in time | ~ |
| | | between the left | |
| | | and right hindpaw | |
| | | strike, divided by | |
| | | the total strike time | |
| Gait | stride_count | Sum of all recorded | strides |
| | | strides in video | |
| Gait | distance_cm_sc | Sum of locomotor activity, | Centimeters |
| | | normalized by time | per second |
| | | spent in open field. | |
| Engineered | dAC | Distance between base | centimeters |
| | | of head and base | |
| | | oftail, normalized by | |
| | | the max dAC recorded | |
| Engineered | dB | The distance between the | centimeters |
| | | mid-back point andthe | |
| | | midpoint of the line AC | |
| Engineered | aABC | The angle between | degrees |
| | | the base of head | |
| | | point, mid-back point, | |
| | | and base of tail point | |
| Engineered | width | Width of the ellipse | centimeters |
| | | fit for the mouse | |
| | | calculated | |
| | | for all frames | |
| Engineered | length | Length of the ellipse | centimeters |
| | | fit for the mouse | |
| | | calculated for | |
| | | all frames. | |
| Engineered | rearpaw | The distance between | centimeters |
| | | rearpaws calculated | |
| | | forall frames | |
| Engineered | rear_count | Sum of rearing | ~ |
| | | bouts in video. | |
| Engineered | avg_rear_len | Average length of | seconds |
| | | rearing bouts in video | |

The angular velocity may be the first derivative of angle of the subject, determined by the vector connecting the subject's tail base to its neck base. The lateral displacement may be the difference between the minimum and maximum values of a reference point's (e.g., nose, tail base and tail tip) perpendicular distance from the subject's displacement vector for a stride for each frame of a stride, normalized by the subject's body length. The limb duty factor may be the amount of time that the paw is in contact with the ground divided by the full stride time, calculated and averaged for each hind paw of the subject. The speed may be determined using the tail base. The step length may be the distance that the right hind paw travels past the previous opposite paw strike. The gait measurements data 122 may include two step lengths: one based on the left hind paw strike, and another based on the right hind paw strike. The step width may be the length of the shortest line segment that connects the right hind paw strike to the line that connects the left hind paw's toe-off location to its subsequent foot strike position. The stride length may be the full distance that the left hind paw travels for a stride, from toe-off to foot-strike. The temporal symmetry may be the difference in time between the left hind paw strike and right hind paw strike, divided by the total strike time. The stride count may be the sum of all strides represented in the duration of the video data 104. The distance covered may be the sum of locomotor activity, normalized by time spent in the open field arena.

As shown in FIG. 1, in some embodiments, the gait and posture analysis component 120 may also generate spinal measurements data 124. Referring to FIG. 2, at a step 208, the gait and posture analysis component 120 may process the point data 112 to determine spinal measurements for the subject. A spinal mobility measurement(s) may be determined for each video frame of the video data 104. The spinal mobility measurements for a video frame may include multiple different measurements. Using the point data 112, the gait and posture analysis component 120 may determine a first distance (dAC) between a head base (point A) of the subject and a tail base (point B) of the subject. In some embodiments, the first distance may be normalized for the subject's body length. Using the point data 112, the gait and posture analysis component 120 may determine a second distance (dB) between a mid-back (point B) of the subject and a midpoint of a line between the head base and the tail base (midpoint of the line AC). Using the point data 112, the gait and posture analysis component 120 may determine an angle (aABC) formed by the head base, the tail base and the mid-back of the subject (points A, B and C). The spinal mobility measurement for a video frame may include the foregoing first distance, second distance and angle. The gait and posture analysis component 120 may determine spinal mobility measurements for each video frame of the video data 104. The spinal measurements data 124 may be a vector or a matrix including the spinal mobility measurements for each video frame of the video data 104.

When the subject spine is straight, the first distance (dAC) and the angle (aABC) may be at their maximum value while the second distance (dB) may be at its minimum value. When the subject spine is bent, the second distance (dB) may be at its maximum value while the first distance (dAC) and the angle (aABC) may be at their minimum values. In determining the visual frailty score 162, the visual frailty analysis component 160 may consider the spinal measurements data 124 for the entire duration of the video. The visual frailty analysis component 160 may be configured to identify that an aged subject may bend its spine to a lesser degree, or less often due to reduced flexibility or spinal mobility. For each of the three spinal mobility measurements, the visual frailty analysis component 160 may determine a mean, a median, a standard deviation, a minimum, and a maximum for all the video frames of the video data 104. In some embodiments, the visual frailty analysis component 160 may identify which video frames are non-gait frames (i.e. in which frames the subject is not in stride/walking). For such non-gait frames, the visual frailty analysis component 160 may, separately, determine a mean, a median, a standard deviation, a minimum, and a maximum. The visual frailty analysis component 160 may be configured to identify a correlation the spinal measurements data 124 and the frailty of the subject. For example, in some cases, the first distance (dAC) median for non-gait frames and the second distance (dB) median for non-gait frames may increase (or decrease) with age.

As shown in FIG. 1, in some embodiments, the gait and posture analysis component 120 may also generate rearing event data 126. Referring to FIG. 2, at a step 210, the gait and posture analysis component 120 may process the point data 112 to determine rearing event data 126 for the subject. In some embodiments, a rearing event may be defined as when the subject's nose (or other body part) passes a threshold boundary/line on a wall of the open field. The gait and posture analysis component 120 may be configured to identify a threshold boundary on the wall, and using the point data 112, may identify when (e.g., in which video frames of the video data 104) the nose of the subject passes/is above the threshold boundary. In other embodiments, a rearing event may be defined differently, for example, as when the subject's paws passes a threshold boundary on a wall, when the subject spends a threshold amount of time in a corner of the open field, when the subject spends a threshold amount of time above a threshold boundary on a wall, etc.

The gait and posture analysis component 120 may be configured to use the coordinates of the boundary between the floor and wall of the open field, with a buffer of some pixels. Whenever the subject's nose point crossed the buffer, this frame may be identified as including/representing a rearing event by the gait and posture analysis component 120. Each uninterrupted series of video frames where the subject exhibits the rearing event may identified by the gait and posture analysis component 120 as a rearing bout. In some embodiments, the gait and posture analysis component 120 may determine the total number of rearing bouts, the average length of the rearing bouts, the number of rearing bouts in the first few minutes of the video (e.g., 5 minutes), and the number of rearing bouts within the next few minutes (e.g., between minutes 5 and 10). The foregoing measurements may be included in the rearing event data 126. The visual frailty analysis component 160 may be configured to identify a correlation between the rearing event data 126 and the frailty of the subject. For example, aged/frailer subjects may rear less (or more).

As shown in FIG. 1, in some embodiments, the gait and posture analysis component 120 may also generate rear paw data 128. Referring to FIG. 2, at a step 212, the gait and posture analysis component 120 may process the point data 112 to determine the rear paw data 128. For each video frame of the video data 104, the gait and posture analysis component 120 may determine a distance between the rear paw coordinates (from the point data 112). The rear paw data 128 may a vector including the foregoing distance for each of the video frames. The visual frailty analysis component 160 may determine a median, a mean, a standard deviation, a maximum and/or a minimum of the rear paw distances for all the video frames. The visual frailty analysis component 160 may be configured to identify a correlation between the rear paw data 128 and the frailty of the subject. For example, the distance between the rear paws of an aged/frailer subject may be less (or more) than a control subject.

As shown in FIG. 1, in some embodiments, the system(s) 105 may determine morphometric data 142 for the subject using ellipse data 132 and the open field analysis component 140. FIG. 3 is a flowchart illustrating a process 300 for determining morphometric data for the subject using ellipse data derived from the video data, according to embodiments of the present disclosure.

At a step 302, the ellipse generator component 130 may process the video data 104 to determine the ellipse data 132. In some embodiments, the ellipse generator component 130 may employ techniques to process the video data 104 to generate a segmentation mask identifying the subject in the video data 104, and then generate an ellipse fit/representation for the subject. The ellipse generator component 130 may employ one or more techniques (e.g., one or more ML models) for object tracking in video/image data, and may configured to identify the subject (e.g., which pixels represent the subject vs. which pixels represent the background). The segmentation mask generated by the ellipse generator 130 may identify subject pixels (a set of pixels) corresponding to the subject, and may identify background pixels (another set of pixels separate and different from the subject pixels) corresponding to the background. Using the segmentation mask, the ellipse generator component 130 may determine the ellipse fit. The ellipse fit may be an ellipse drawn around the subject's body. For a different type of subject, the system(s) 105 may be configured to determine a different shape fit/representation (e.g., a circle fit, a rectangle fit, a square fit, etc.). The ellipse generator component 130 may determine the ellipse fit as a subset of the subject pixels. The ellipse data 132 may include this subset of pixels corresponding to the ellipse fit. The ellipse generator component 130 may determine an ellipse fit for each video frame of the video data 104. The ellipse data 132 may be a vector or a matrix of the ellipse fit pixels for all the video frames of the video data 104.

In some embodiments, the ellipse fit for the subject may define some parameters of the subject. For example, the ellipse fit may correspond to the subject's location, and may include coordinates (e.g., x and y) representing a pixel location (e.g., the center of the ellipse) of the subject in a video frame(s) of the video data 104. The ellipse fit may correspond to a major axis length and a minor axis length of the subject. The ellipse fit may include a sine and cosine of a vector angle of the major axis. The angle may be defined with respect to the direction of the major axis. The major axis may extend from a tip of the subject's head or nose to an end of the subject's body such as a tail base. In some embodiments, the ellipse data 132 may include the foregoing measurements for all video frames of the video data 104.

In some embodiments, the ellipse generator component 130 may use an encoder-decoder architecture to determine the segmentation mask from the video data 104. In some embodiments, the ellipse generator component 130 may use a neural network model to determine the ellipse fit from the video data 104.

The ellipse data 132 may also include a confidence score(s) of the ellipse generator component 130 in determining the ellipse fit for the video frame. The ellipse data 132 may alternatively include a probability or likelihood of the ellipse fit corresponding to the subject.

In some embodiments, the ellipse generator component 130 may determine an ellipse fit for the subject for each video frame of the video data 104. The ellipse fit may be represented as a set of pixels defining the ellipse around the subject. The ellipse data 132 may be a vector or a matrix including the ellipse fit data for all of the video frames.

At a step 304, the open field analysis component 140 may process the ellipse data 132 to determine the morphometric data 142. The morphometric data 142 may correspond to a body composition (e.g., shape, size, length, weight, etc.) of the subject. The open field analysis component 140 may determine an estimated length and estimated width of the subject using a length of a major axis and a minor axis of the ellipse fit (from the ellipse data 132) for the subject. The open field analysis component 140 may determine the major and minor axis for the ellipse fit for each video frame of the video data 104. In some embodiments, the open field analysis component 140 may determine a median, a mean, a standard deviation, a maximum and/or a minimum for the length of the major and/or minor axis for all the video frames of the video data 104. The open field analysis component 140 may estimate the length and width of the subject using one or more of the foregoing calculations. In some embodiments, the morphometric data 142 may include the estimated length and width of the subject. The morphometric data 142 may additionally or alternatively include the length of the major and minor axis for each ellipse fit for each video frame of the video data 104.

The visual frailty analysis component 160 may be configured to identify a correlation between the morphometric data 142 and the frailty of the subject. For example, changes in body composition and fat distribution may be observed with aging of the subject.

In some embodiments, the open field analysis component 140 may determine other data that may be used by the visual frailty analysis component 160. For example, the open field analysis component 140 may use the ellipse data 132 (e.g., a center pixel/location of the ellipse) to determine in which video frames the subject is in the center of the open field arena, and may determine the amount of time the subject spends in the center of the open field arena for the duration of the video. As another example, the open field analysis component 140 may use the ellipse data 132 to determine in which video frames the subject is along the wall of the open field arena, and may determine the amount of time the subject spends in the periphery (along the wall) for the duration of the video. As yet another example, the open field analysis component 140 may use the ellipse data 132 to determine in which video frames the subject is in a corner of the open field arena, and may determine the amount of time the subject spends in the corner(s) for the duration of the video. As yet another example, the open field analysis component 140 may use the ellipse data 132 to determine an average distance the subject is located from the center of the open field arena during the video. As yet another example, the open field analysis component 140 may use the ellipse data 132 to determine an average distance the subject is located from the periphery/walls of the open field arena during the video. As yet another example, the open field analysis component 140 may use the ellipse data 132 to determine an average distance the subject is located from the corners of the open field arena during the video.

As shown in FIG. 1, in some embodiments, the system(s) 105 may determine behavior data 152 for the subject using the grooming behavior analysis component 150. FIG. 4 is a flowchart illustrating a process 400 for determining the behavior data 152 for the subject using the video data 104, according to embodiments of the present disclosure. At a step 402, the grooming behavior analysis component 150 may process the video data 104 to determine the behavior data 152 for the subject. The grooming behavior analysis component 150 may be configured to identify the video frames of the video data 104 where the subject exhibits grooming behavior, which may include at least one of paw licking, unilateral face wash, bilateral face wash, and flank licking. In some embodiments, the grooming behavior analysis component 150 may process the video data 104 using more than one ML model to generate multiple predictions regarding whether one or more frames in the video data 104 represent the subject exhibiting a defined behavior. These ML models may be configured using training data that includes video capturing movements of a subject(s), where the training data includes labels for each video frame identifying whether the subject is exhibiting grooming behavior or not. Such ML models may be configured using a large training dataset.

Each of the ML models of the grooming behavior analysis component 150 may be configured using different initialization parameters or settings, so that the ML models may have variations in terms of certain model parameters (such as, learning rate, weights, batch size, etc.), therefore, resulting in different predictions (regarding the subject's grooming behavior) when processing the same video frames.

The grooming behavior analysis component 150 may also process different representations of the video data 104. The grooming behavior analysis component 150 may determine different representations of the video data 104 by modifying the orientation of the video. For example, one orientation may be determined by rotating the video by 90 degrees left, another orientation may be determined by rotating the video by 90 degrees right, and yet another orientation may be determined by reflecting the video along a horizontal or vertical axis. The grooming behavior analysis component 150 may process the video frames in the originally-captured orientation and the other different generated orientations. Based on processing different orientations, the grooming behavior analysis component 150 may generate different predictions regarding the subject's grooming behavior. The grooming behavior analysis component 150 may use the different predictions, determined as described above, to make a final determination regarding whether the subject is exhibiting grooming behavior in the video frame(s).

The final determination may be outputted at the behavior data 152. The behavior data 152 may be a vector or a set of values indicating whether the subject exhibiting grooming behavior in a particular video frame. For example, the behavior data 152 may include Boolean values (e.g., 1 or 0; true or false; yes or no; etc.) for each video frame indicating whether or not the subject exhibited grooming behavior. As another example, the behavior data 152 may alternatively or additionally include a score (e.g., a confidence score, a probability score, etc.) corresponding to whether the subject exhibited grooming behavior in the particular video frame.

The grooming behavior analysis component 150 may determine multiple sets of frames using the video data 104, where the different sets (e.g., at least four sets) may represent a different orientation of the video data. A first set(s) of frames may be the original orientation of video data 104 captured by the image capture device 101. A rotated set(s) of frames may be a rotated orientation of the video data 104, for example, the first set(s) of frames may be rotated 90 degrees left to generate the rotated set(s) of frames. A reflected set(s) of frames may be a reflected orientation of the video data 104, for example, the first set(s) of frames may be reflected across a horizontal axis (or rotated by 180 degrees) to generate the reflected set of frames. Another rotated set(s) of frames may be another rotated orientation of the video data 104, for example, the first set of frames may be rotated 90 degrees right to generate the other rotated set(s) of frames. In other embodiments, sets of frames may be generated by manipulating the original set(s) of frames in other ways (e.g., reflecting across a vertical axis, rotated by another number of degrees, etc.). In other embodiments, more or fewer orientations of the video data 104 may be processed by the grooming behavior analysis component 150.

The grooming behavior analysis component 150 may employ at least four ML models. As part of processing the video data 104, the grooming behavior analysis component 150 may process the different foregoing sets of frames using the same ML model to generate different predictions. For example, a first ML model may process the first set(s) of frames to generate a first prediction representing a probability or likelihood of the subject exhibiting grooming behavior during the first set of frames. The first ML model may process the rotated set(s) of frames to generate a second prediction representing a probability or likelihood of the subject exhibiting grooming behavior during the rotated set(s) of frames. The first ML model may process the reflected set(s) of frames to generate a third prediction representing a probability or likelihood of the subject exhibiting the behavior during the reflected set(s) of frames. The first ML model may process the other rotated set(s) of frames to generate a fourth prediction representing a probability or likelihood of the subject exhibiting the behavior during the other rotated set(s) of frames. In this manner, the same ML model may process different orientations of the video data 104 to generate different predictions for the same captured subject movements.

As part of further processing the video data 104, the grooming behavior analysis component 150 may process the different foregoing sets of frames using another ML model to generate more predictions. For example, a second ML may process the first set(s) of frames to generate a fifth prediction representing a probability or likelihood of the subject exhibiting the behavior during the first set(s) of frames. The second ML model may process the rotated set(s) of frames to generate a sixth prediction representing a probability or likelihood of the subject exhibiting the behavior during the rotated set(s) of frames. The second ML model may process the reflected set(s) of frames to generate a seventh prediction data representing a probability or likelihood of the subject exhibiting the behavior during the reflected set(s) of frames. The second ML model may process the other rotated set(s) of frames to generate an eighth prediction representing a probability or likelihood of the subject exhibiting the behavior during the video representing in the other rotated set(s) of frames. In this manner, another ML model may process different orientations of the video data 104 to generate additional predictions for the same captured subject movements. The probabilities may be a value in the range of 0.0 to 1.0, or a value in the range of 0 to 100, or another numerical range.

Each of the different predictions (e.g., the eight predictions) may be a data vector including multiple probabilities (or scores), each probability corresponding to a frame of the set(s) respectively, where each probability indicates a likelihood of the subject exhibiting grooming behavior in the corresponding frame. For example, the prediction may include a first probability corresponding to a first frame of the video data 104, a second probability corresponding to a second frame of the video data 104, and so on.

In some embodiments, the set(s) of frames may include a number of video frames (e.g., 16 frames), each frame being a duration of video for a time period (e.g., 30 milliseconds, 30 seconds, etc.). Each of the ML models may be configured to process the set of frames to determine a probability of the subject exhibiting grooming behavior in the last frame of the set of frames. For example, if there are 16 frames in the set of frames, then the output of the ML model indicates whether or not the subject is exhibiting grooming behavior in the 16^{th} frame of the set of frames. The ML models may be configured to use context information from the other frames in the set of frames to make the prediction of the last frame. In other embodiments, the output of the ML models may determine a probability of the subject exhibiting grooming behavior in another frame (e.g., middle frame; 8^{th} frame; first frame; etc.) of the set of frames.

In some embodiments, the grooming behavior analysis component 150 may generate 32 different predictions corresponding to a frame by processing four different orientations/set of frames using four different ML models.

The grooming behavior analysis component 150 may include an aggregation component to process the different predictions determined by the different ML models using the different sets of frames to determine the final prediction indicated in the behavior data 152. The aggregation component may be configured to merge, aggregate or otherwise combine the different predictions (e.g., the eight predictions described above) to determine the behavior data 152.

In some embodiments, the aggregation component may average the probabilities for the respective frames, and the behavior data 152 may be a data vector of averaged probabilities for each frame in the video data 104. In some embodiments, the grooming behavior analysis component 150 may determine a behavior label for a frame (or a number of frames) based on the frame's corresponding averaged probability satisfying a condition (e.g., if the probability is above a threshold probability/value), where the behavior label may be a Boolean value indicating whether or not the subject exhibited grooming behavior.

In other embodiments, the aggregation component may sum the probabilities for the respective frames, and the behavior data 152 may be a data vector of summed probabilities for each frame in the video data 104. In some embodiments, the grooming behavior analysis component 150 may determine the behavior label for a frame based on the frame's corresponding summed probability satisfying a condition (e.g., if the probability is above a threshold probability/value).

In some embodiments, the aggregation component may be configured to select the maximum value (e.g., the highest probability) from the predictions for the respective frame as the final prediction for the frame. In other embodiments, the aggregation component may be configured to determine a median value from the predictions as the final prediction for the frame.

In some embodiments, another component may be configured in a similar manner as the grooming behavior analysis component 150 to detect the subject exhibiting another predefined behavior. This other component may use more than ML model to process the video data 104. These ML models may be configured to detect a particular behavior using training data that includes video capturing movements of a subject(s), where the training data includes labels for each video frame identifying whether the subject is exhibiting the particular behavior or not. Such ML models may be configured using a large training dataset. Based on the configurations of the ML models, the can be configured to detect different behaviors.

In other embodiments, the grooming behavior analysis component 150 may employ other techniques for determining the behavior data 152.

The behavior data 152 may also include a number of frames/times the subject exhibits grooming for the duration of the video, a length of each grooming bout (consecutive video frames in which the subject is grooming), an average length of the grooming bouts, a number of grooming bouts for the duration of the video, and other metrics.

The visual frailty analysis component 160 may be configured to identify a correlation between the behavior data 152 and the frailty of the subject. For example, an aged/frail subject may groom less (or more) than a control subject.

FIG. 5 is a flowchart illustrating a process for determining a visual frailty score using one or more of the data determined according to the processes of FIGS. 2-4, according to embodiments of the present disclosure. At a step 502, the visual frailty analysis component 160 may process one or more of the determined data 122, 124, 126, 128, 142, 152 using one or more ML models. In some embodiments, the visual frailty analysis component 160 may employ one ML model to process all the features/data 122, 124, 126, 128, 142, 152. In other embodiments, the visual frailty analysis component 160 may employ different/separate ML models to process each of the data 122, 124, 126, 128, 142, 152. For example, a first ML model may be used to process the gait measurements data 122, a second ML model may be used to process the spinal measurements data 124, and so on. In yet another embodiment, the visual frailty analysis component 160 may employ different/separate ML models to process the data 122, 124, 126, 128, 142, 152 based on how the data is derived and/or which component generated the data. For example, a first ML model may be used to process the gait measurements data 122, the spinal measurements data 124, the rearing event data 126 and the rear paw data 128 determined by the gait and posture analysis component 120; while a second ML model may be used to process the morphometric data 142.

In some embodiments, the visual frailty analysis component 160 may select different features/data, based on the subject's age, gender, strain, and/or other characteristics, for determining the visual frailty score 162.

At a step 504, the visual frailty analysis component 160 may determine the visual frailty score 162 for the subject. In some embodiments, the visual frailty analysis component 160 may determine different/multiple initial frailty scores based on processing the different types of data inputted to the visual frailty analysis component 160, and may then aggregate the different/multiple frailty scores to determine a final visual frailty score 162. In aggregating the results of processing the different types of data, the visual frailty analysis component 160 may use a weighted sum or a weighted average technique, where different types of data may have a different corresponding weight. For example, results of processing the morphometric data 142 may be associated with a first weight, while the results of processing the spinal measurements data 124 may be associated with another weight.

The visual frailty score 162 may be a numerical value in a predetermined range. For example, the visual frailty score 162 may be a value between 0 to 1; 0 to 10; 1 to 27; 0 to 100; etc.

In other embodiments, the visual frailty analysis component 160 may use another ML model to aggregate/combine the results of processing the different types of data to determine the visual frailty score 162. In yet other embodiments, the visual frailty analysis component 160 may use a rules-based engine to aggregate/combine the results of processing the different types of data to determine the visual frailty score 162.

In yet other embodiments, the visual frailty analysis component 160 may be configured to use the point data 112 and/or the ellipse data 132 and may take into consideration the confidence of the point tracker component 110 and/or the ellipse generator component 130 in determining the visual frailty score 162.

In some embodiments, the visual frailty analysis component 160 may determine the visual frailty score 162 based on a comparison/evaluation of the data 122, 124, 126, 128, 142, 152 with respect to some stored/control data. The visual frailty analysis component 160 may select the stored/control data based on the age, gender, strain and/or characteristics of the subject.

In some embodiments, the visual frailty analysis component 160 may determine the visual frailty score 162 based on which factors/features/data are visible/evident/detected for the subject. The visual frailty analysis component 160 may determine further factors using the data 122, 124, 126, 128, 142 and 152 for the subject. The visual frailty analysis component 160 may sum the number of factors detected, and divide the sum with the number of total factors considered. For example, the visual frailty analysis component 160 may determine, using the gait measurements data 122, that the subject has gait defects, and may determine, using the morphometric data 142, that the subject has gained weight. The detection of gait defects and weight gain may be two factors detected for the subject out of ten potential factors. Based on this, the visual frailty analysis component 160 may determine the visual frailty score 162 to be 0.2 (2/10).

In some embodiments, the visual frailty analysis component 160 may employ multiple different types of models/algorithms to process the different types of data. For example, the visual frailty analysis component 160 may include one or more of: a linear regression model, a penalized linear regression model, a random forest, a support vector machine, a gradient boosting model, an extreme gradient boosting model, and a neural network.

Although FIG. 1 shows certain types of data, it should be understood that the visual frailty analysis component 160 may process additional or different types of data to determine the visual frailty score 162.

In some embodiments, the data shown in FIG. 1 may be determined by different components and/or using different data extracted from the video data 104. For example, morphometric data may be determined using the point data 112. As another example, rear paw data may be determined using the ellipse data 132. As another example, behavior data may be determined using the point data 112.

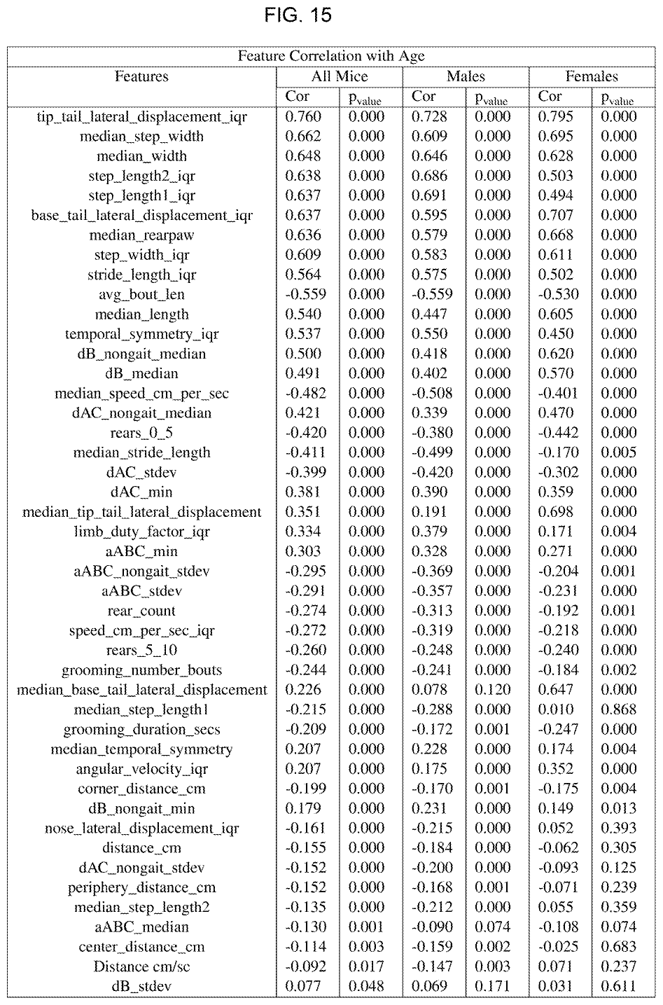

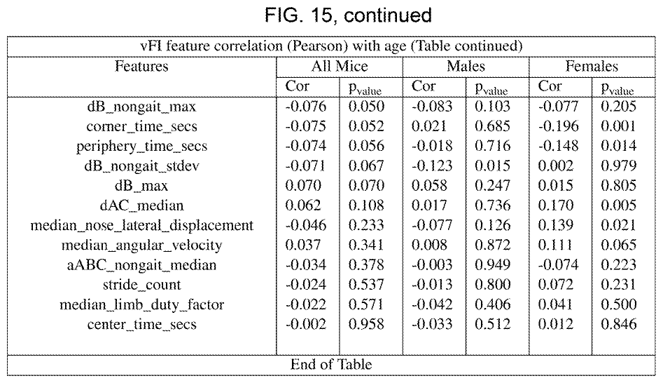

In some embodiments, the visual frailty analysis component 160 may be configured/trained using data corresponding to manually generated frailty scores. The manual frailty scores may be generated by observers/scorers by observing videos of multiple different subjects. Some factors considered by the observers in generating the manual frailty score are listed in FIG. 15. A video of a subject may be annotated/labeled with the corresponding manual frailty score and/or the factors detected for the subject in generating the manual frailty score. The visual frailty analysis component 160 may be configured using such annotated videos. The factors/data considered in generating the manual frailty score may be different than the factors/data used in generating the visual frailty score 162.

### Subjects

Some aspects of the invention include determining a visual frailty score for a subject. As used herein, a the term “subject” may refer to a human, non-human primate, cow, horse, pig, sheep, goat, dog, cat, pig, bird, rodent, or other suitable vertebrate or invertebrate organism. In certain embodiments of the invention, a subject is a mammal and in certain embodiments of the invention, a subject is a human. In some embodiments, a subject used in method of the invention is a rodent, including but not limited to a: mouse, rat, gerbil, hamster, etc. In some embodiments of the invention, a subject is a normal, healthy subject and in some embodiments, a subject is known to have, at risk of having, or suspected of having a disease or condition associated with frailty. A disease associated with frailty may include clinical characteristics/symptoms such as: muscle weakness, loss of balance, abnormal muscle fatigue, muscle wasting, etc. In certain embodiments of the invention, a subject is an animal model for a disease or condition associated with frailty. For, example though not intended to be limiting, in some embodiments of the invention a subject is a mouse that is an animal model for aging, a characteristic of frailty such as one or more of muscle weakness, loss of balance, abnormal muscle fatigue, muscle wasting, etc.

As a non-limiting example, a subject assessed with a method and system of the invention may be a subject that is an animal model for a condition such as a model for one or more of: aging, frailty, a neurodegenerative illness, a neuromuscular illness, muscle trauma, ALS, Parkinson's disease, multiple sclerosis, muscular dystrophy, etc. Such conditions may also be referred to herein as “activity disorders”.

In some embodiments of the invention, a subject is a wild-type subject. As used herein the term “wild-type” means to the phenotype and/or genotype of the typical form of a species as it occurs in nature. In certain embodiments of the invention a subject is a non-wild-type subject, for example, a subject with one or more genetic modifications compared to the wild-type genotype and/or phenotype of the subject's species. In some instances, a genotypic/phenotypic difference of a subject compared to wild-type results from a hereditary (germline) mutation or an acquired (somatic) mutation. Factors that may result in a subject exhibiting one or more somatic mutations include but are not limited to: environmental factors, toxins, ultraviolet radiation, a spontaneous error arising in cell division, a teratogenic event such as but not limited to radiation, maternal infection, chemicals, etc.

In certain embodiments of methods of the invention, a subject is a genetically modified organism, also referred to as an engineered subject. An engineered subject may include a pre-selected and/or intentional genetic modification and as such exhibits one or more genotypic and/or phenotypic traits that differ from the traits in a non-engineered subject. In some embodiments of the invention, routine genetic engineering techniques can be used to produce an engineered subject that exhibits genotypic and/or phenotypic differences compared to a non-engineered subject of the species. As a non-limiting example, a genetically engineered mouse in which a functional gene product is missing or is present in the mouse at a reduced level and a method or system of the invention can be used to assess the genetically engineered mouse phenotype, and the results may be compared to results obtained from a control (control results).

In some embodiments of the invention, a subject may be monitored using a visual frailty determining method or system of the invention and the presence or absence of an activity disorder or condition can be detected. In certain embodiments of the invention, a test subject that is an animal model of an activity and/or movement condition may be used to assess the test subject's response to the condition. In addition, a test subject that is an animal model of a movement and/or activity condition may be administered a candidate therapeutic agent or method, monitored using a gait monitoring method and/or system of the invention and results can be used to determine an efficacy of the candidate therapeutic agent to treat the condition. The terms “activity” and “action” may be used interchangeably herein.

As described elsewhere here, methods and systems of the invention may be configured to determine a visual frailty score of a subject, regardless of the subject's physical characteristics. In some embodiments of the invention, one or more physical characteristics of a subject may be pre-identified characteristics. For example, though not intended to be limiting, a pre-identified physical characteristic may be one or more of: a body shape, a body size, a coat color, a gender, an age, and a phenotype of a disease or condition.

### Diseases and Disorders

Methods and systems of the invention can be used to assess frailty, activity and/or behavior of a subject known to have, suspected of having, or at risk of having a disease or condition associated with frailty. It will be understood that in some instances frailty is an age-related condition. For example, a subject may be a geriatric subject and/or may be an animal model for a geriatric condition. In certain embodiments of the invention, frailty is not associated with aging but may be associated with a disease or condition that is not considered to be a geriatric condition. For example, muscle weakness may be a characteristic assessed using a method of the invention and it might be exhibited by a young subject; a subject that is not an animal model of a geriatric condition; a subject that is an animal model of a geriatric condition; or a geriatric subject. In some embodiments, the disease and/or condition is one associated with an abnormally reduced level of an activity or behavior such as movement, muscle use, stamina, etc. In anon-limiting example, a test subject that may be subject with muscle wasting and weakness or subject that is an animal model of a condition that manifests with muscle wasting and/or weakness, etc. In each case, a method of the invention can be used to assess the subject to determine the frailty status of the subject. Results of assessing the test subject can be compared to control results of the assessment, non-limiting examples of control subjects are: subjects that do not have the model disease or condition, subjects that do not have muscle wasting, subjects that do not have muscle weakness, etc. A control standard may also be obtained from a plurality of subjects without the condition, etc. Differences in the results of the test subject and the control can be compared. Some embodiments of methods of the invention can be used to identify subjects that have a disease or condition that is associated with frailty.

Onset, progression, and/or regression of a disease or a condition associated with frailty can also be assessed and tracked using embodiments of methods of the invention. For example in certain embodiments of methods of the invention, 2, 3, 4, 5, 6, 7, or more assessments of a subject using a method of the invention are carried out at different times. A comparison of two or more of the results of the assessments made at different times can show differences in the frailty status (e.g. frailty level) of the subject. An increase in a determined level and/or characteristic of frailty exhibited by the subject may indicate onset and/or progression in the subject of a disease or condition associated with frailty. A decease in a determined level or type of an activity may indicate regression in the subject of a disease or condition associated with the assessed activity. A determination that an activity has ceased in a subject may indicate the cessation in the subject of the disease or condition associated with the assessed activity.

Certain embodiments of methods of the invention can be used to assess efficacy of a therapy to treat a disease or condition associated with frailty. For example, a test subject may be administered a candidate therapy and methods of the invention used to determine in the subject, a presence or absence of a change in frailty. A reduction in frailty determined in the subject following administration of a candidate therapy may indicate efficacy of the candidate therapy against the frailty-associated disease or condition.

As indicated elsewhere herein, a visual frailty analysis method of the invention may be used to assess a disease, condition or aging in a subject and may also be used to assess animal models of diseases, conditions and aging. Numerous different animal models for diseases, conditions and aging are known in the art, including but not limited to numerous mouse models. A subject assessed with a system and/or method of the invention may be a subject that is an animal model for a disease or condition such as a model for a disease or condition such as, but not limited to: neurodegenerative disorders, neuromuscular disorders, ALS, depression, a hyperkinetic disorder, an anxiety disorder, a muscle wasting disease, a muscle injury, a developmental disorder, Parkinson's disease, a physical injury, etc. Additional models of diseases and disorders that may be assessed using a method and/or system of the invention are known in the art, see for example: Dawson, T. M., et al., Neuron June 10; 66(5):646-61 (2010); Cenci, M. A. & A. Bjorklund Prog Brain Res. 252:27-59 (2020); Fleming, S. M. et al., NeuroRx July; 2(3):495-503 (2005); Farshim, P. P, & G. P. Bates Methods Mol. Biol. 1780:97-120 (2018); Nair, R. R. et al., Mamm Genome. August; 30(7-8):173-191 (2019); Sukoff Rizzo, S. J. & J. N. Crawley Annu Rev. Anim Biosci. February 8; 5:371-389 (2017); Trancikova, A. et al., Prog Mol Biol Transl Sci. 100:419-82 (2011); Russell, V. A. Curr Protoc Neurosci. January; Chapter 9: Unit 9.35 (2011); Leo, D. & R. R. Gainetdinov Cell Tissue Res. October; 354(1):259-71 (2013); Campos, A. C. et al., Braz J. Psychiatry 35 Suppl 2:S101-11 (2013); and Szechtman. J. et al. Neurosci Biobehav Rev. May; 76(Pt B); 254-279 (2017), the contents of which are incorporated herein by reference in their entirety.

In addition to testing subjects with known diseases or disorders, methods of the invention may also be used to assess new genetic variants, such as engineered organisms. Thus, methods of the invention can be used to assess an engineered organism for one or more characteristics of a disease or condition. In this manner, new strains of organisms, such as new mouse strains can be assessed and the results used to determine whether the new strain is an animal model for a disease or disorder.

### Example Devices and Systems

One or more of the ML models of the automated visual frailty system 100 may take many forms, including a neural network. A neural network may include a number of layers, from an input layer through an output layer. Each layer is configured to take as input a particular type of data and output another type of data. The output from one layer is taken as the input to the next layer. While values for the input data/output data of a particular layer are not known until a neural network is actually operating during runtime, the data describing the neural network describes the structure, parameters, and operations of the layers of the neural network.

One or more of the middle layers of the neural network may also be known as the hidden layer. Each node of the hidden layer is connected to each node in the input layer and each node in the output layer. In the case where the neural network comprises multiple middle networks, each node in a hidden layer will connect to each node in the next higher layer and next lower layer. Each node of the input layer represents a potential input to the neural network and each node of the output layer represents a potential output of the neural network. Each connection from one node to another node in the next layer may be associated with a weight or score. A neural network may output a single output or a weighted set of possible outputs.

In one aspect, the neural network may be constructed with recurrent connections such that the output of the hidden layer of the network feeds back into the hidden layer again for the next set of inputs. Each node of the input layer connects to each node of the hidden layer. Each node of the hidden layer connects to each node of the output layer. The output of the hidden layer is fed back into the hidden layer for processing of the next set of inputs. A neural network incorporating recurrent connections may be referred to as a recurrent neural network (RNN).

In some embodiments, the neural network may be a long short-term memory (LSTM) network. In some embodiments, the LSTM may be a bidirectional LSTM. The bidirectional LSTM runs inputs from two temporal directions, one from past states to future states and one from future states to past states, where the past state may correspond to characteristics for the video data for a first time frame and the future state may corresponding to characteristics for the video data for a second subsequent time frame.

Processing by a neural network is determined by the learned weights on each node input and the structure of the network. Given a particular input, the neural network determines the output one layer at a time until the output layer of the entire network is calculated.

Connection weights may be initially learned by the neural network during training, where given inputs are associated with known outputs. In a set of training data, a variety of training examples are fed into the network. Each example typically sets the weights of the correct connections from input to output to 1 and gives all connections a weight of 0. As examples in the training data are processed by the neural network, an input may be sent to the network and compared with the associated output to determine how the network performance compares to the target performance. Using a training technique, such as back propagation, the weights of the neural network may be updated to reduce errors made by the neural network when processing the training data.

Various machine learning techniques may be used to train and operate models to perform various steps described herein, such as determining point data, determining ellipse data, determining behavior data, determining visual frailty scores, etc. Models may be trained and operated according to various machine learning techniques. Such techniques may include, for example, neural networks (such as deep neural networks and/or recurrent neural networks), inference engines, trained classifiers, etc. Examples of trained classifiers include Support Vector Machines (SVMs), neural networks, decision trees, AdaBoost (short for “Adaptive Boosting”) combined with decision trees, and random forests. Focusing on SVM as an example, SVM is a supervised learning model with associated learning algorithms that analyze data and recognize patterns in the data, and which are commonly used for classification and regression analysis. Given a set of training examples, each marked as belonging to one of two categories, an SVM training algorithm builds a model that assigns new examples into one category or the other, making it a non-probabilistic binary linear classifier. More complex SVM models may be built with the training set identifying more than two categories, with the SVM determining which category is most similar to input data. An SVM model may be mapped so that the examples of the separate categories are divided by clear gaps. New examples are then mapped into that same space and predicted to belong to a category based on which side of the gaps they fall on. Classifiers may issue a “score” indicating which category the data most closely matches. The score may provide an indication of how closely the data matches the category.

In order to apply the machine learning techniques, the machine learning processes themselves need to be trained. Training a machine learning component such as, in this case, one of the first or second models, requires establishing a “ground truth” for the training examples. In machine learning, the term “ground truth” refers to the accuracy of a training set's classification for supervised learning techniques. Various techniques may be used to train the models including backpropagation, statistical learning, supervised learning, semi-supervised learning, stochastic learning, or other known techniques.

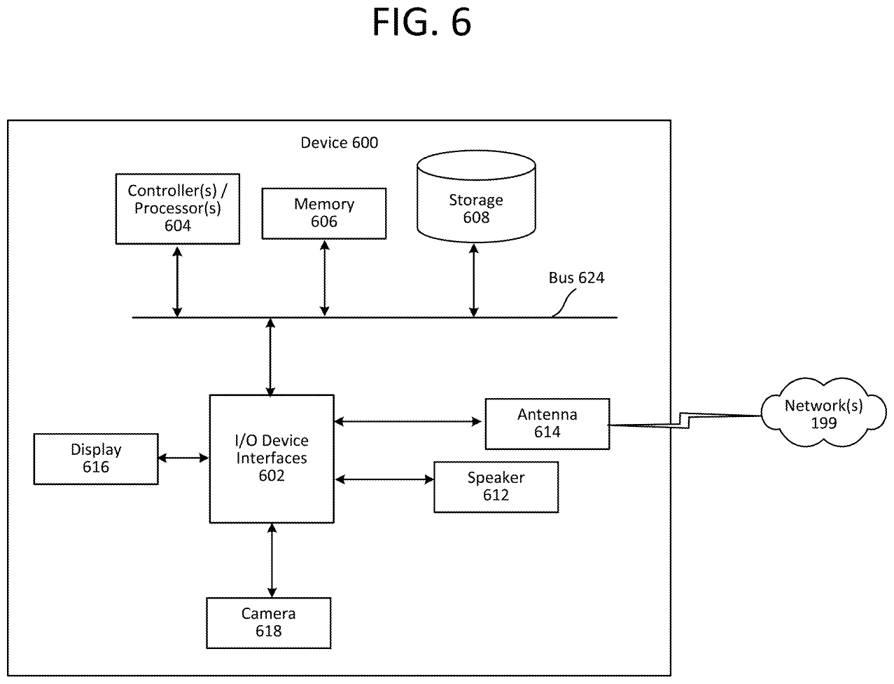

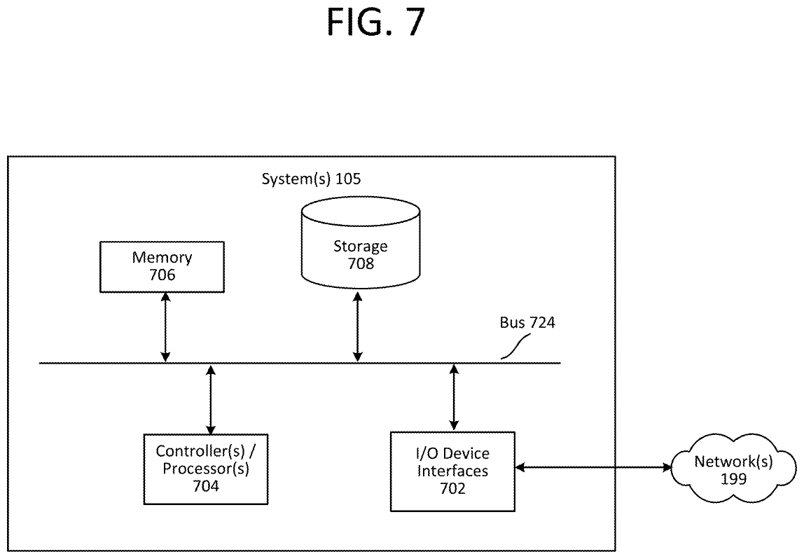

FIG. 6 is a block diagram conceptually illustrating a device 600 that may be used with the system. FIG. 7 is a block diagram conceptually illustrating example components of a remote device, such as the system(s) 105, which may assist processing of video data, identifying subject behavior, etc. A system(s) 105 may include one or more servers. A “server” as used herein may refer to a traditional server as understood in a server/client computing structure but may also refer to a number of different computing components that may assist with the operations discussed herein. For example, a server may include one or more physical computing components (such as a rack server) that are connected to other devices/components either physically and/or over a network and is capable of performing computing operations. A server may also include one or more virtual machines that emulates a computer system and is run on one or across multiple devices. A server may also include other combinations of hardware, software, firmware, or the like to perform operations discussed herein. The server(s) may be configured to operate using one or more of a client-server model, a computer bureau model, grid computing techniques, fog computing techniques, mainframe techniques, utility computing techniques, a peer-to-peer model, sandbox techniques, or other computing techniques.

Multiple systems 105 may be included in the overall system of the present disclosure, such as one or more systems 105 for performing point/body part tracking, one or more systems 105 for ellipse fit/representation determination, one or more systems 105 for behavior classification, one or more systems 150 for determining the visual frailty score, etc. In operation, each of these systems may include computer-readable and computer-executable instructions that reside on the respective device 105, as will be discussed further below.

Each of these devices (600/105) may include one or more controllers/processors (604/704), which may each include a central processing unit (CPU) for processing data and computer-readable instructions, and a memory (606/706) for storing data and instructions of the respective device. The memories (606/706) may individually include volatile random access memory (RAM), non-volatile read only memory (ROM), non-volatile magnetoresistive memory (MRAM), and/or other types of memory. Each device (600/105) may also include a data storage component (608/708) for storing data and controller/processor-executable instructions. Each data storage component (608/708) may individually include one or more non-volatile storage types such as magnetic storage, optical storage, solid-state storage, etc. Each device (600/105) may also be connected to removable or external non-volatile memory and/or storage (such as a removable memory card, memory key drive, networked storage, etc.) through respective input/output device interfaces (602/702).

Computer instructions for operating each device (600/105) and its various components may be executed by the respective device's controller(s)/processor(s) (604/704), using the memory (606/706) as temporary “working” storage at runtime. A device's computer instructions may be stored in a non-transitory manner in non-volatile memory (606/706), storage (608/708), or an external device(s). Alternatively, some or all of the executable instructions may be embedded in hardware or firmware on the respective device in addition to or instead of software.

Each device (600/105) includes input/output device interfaces (602/702). A variety of components may be connected through the input/output device interfaces (602/702), as will be discussed further below. Additionally, each device (600/105) may include an address/data bus (624/724) for conveying data among components of the respective device. Each component within a device (600/105) may also be directly connected to other components in addition to (or instead of) being connected to other components across the bus (624/724).

Referring to FIG. 6, the device 600 may include input/output device interfaces 602 that connect to a variety of components such as an audio output component such as a speaker 612, a wired headset or a wireless headset (not illustrated), or other component capable of outputting audio. The device 600 may additionally include a display 616 for displaying content. The device 600 may further include a camera 618.

Via antenna(s) 614, the input/output device interfaces 602 may connect to one or more networks 199 via a wireless local area network (WLAN) (such as WiFi) radio, Bluetooth, and/or wireless network radio, such as a radio capable of communication with a wireless communication network such as a Long Term Evolution (LTE) network, WiMAX network, 3G network, 4G network, 5G network, etc. A wired connection such as Ethernet may also be supported. Through the network(s) 199, the system may be distributed across a networked environment. The I/O device interface (602/702) may also include communication components that allow data to be exchanged between devices such as different physical servers in a collection of servers or other components.

The components of the device(s) 600 or the system(s) 105 may include their own dedicated processors, memory, and/or storage. Alternatively, one or more of the components of the device(s) 600, or the system(s) 105 may utilize the I/O interfaces (602/702), processor(s) (604/704), memory (606/706), and/or storage (608/708) of the device(s) 600, or the system(s) 105, respectively.

As noted above, multiple devices may be employed in a single system. In such a multi-device system, each of the devices may include different components for performing different aspects of the system's processing. The multiple devices may include overlapping components. The components of the device 600, and the system(s) 105, as described herein, are illustrative, and may be located as a stand-alone device or may be included, in whole or in part, as a component of a larger device or system.

The concepts disclosed herein may be applied within a number of different devices and computer systems, including, for example, general-purpose computing systems, video/image processing systems, and distributed computing environments.

The above aspects of the present disclosure are meant to be illustrative. They were chosen to explain the principles and application of the disclosure and are not intended to be exhaustive or to limit the disclosure. Many modifications and variations of the disclosed aspects may be apparent to those of skill in the art. Persons having ordinary skill in the field of computers and speech processing should recognize that components and process steps described herein may be interchangeable with other components or steps, or combinations of components or steps, and still achieve the benefits and advantages of the present disclosure. Moreover, it should be apparent to one skilled in the art, that the disclosure may be practiced without some or all of the specific details and steps disclosed herein.

Aspects of the disclosed system may be implemented as a computer method or as an article of manufacture such as a memory device or non-transitory computer readable storage medium. The computer readable storage medium may be readable by a computer and may comprise instructions for causing a computer or other device to perform processes described in the present disclosure. The computer readable storage medium may be implemented by a volatile computer memory, non-volatile computer memory, hard drive, solid-state memory, flash drive, removable disk, and/or other media. In addition, components of system may be implemented as in firmware or hardware.

### EXAMPLES

### Example 1. Development of Automated Visual Frailty Indexing

### Methods

### Mice

C57BL/6J mice were obtained from the Nathan Shock Center at the Jackson Laboratory.

### Open Field Assay and Frailty Indexing

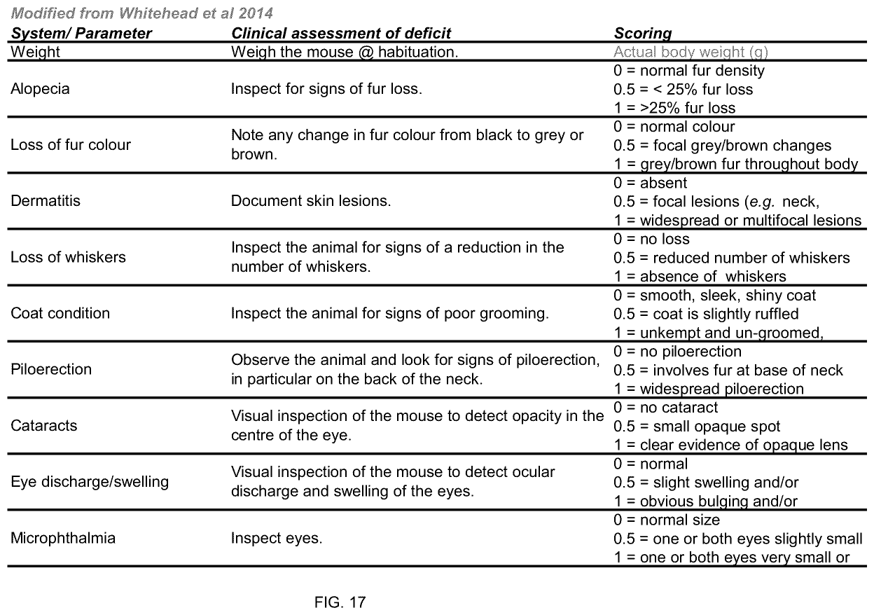

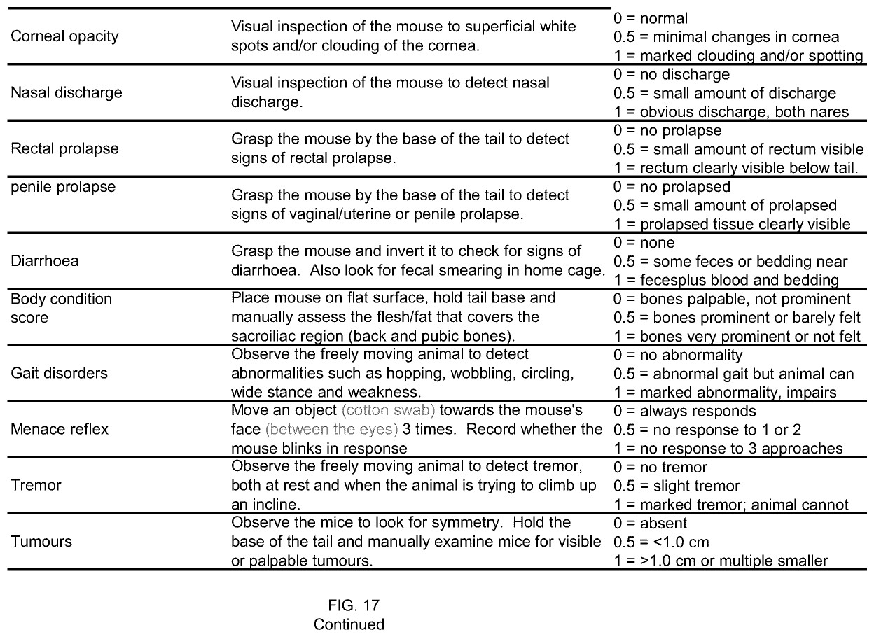

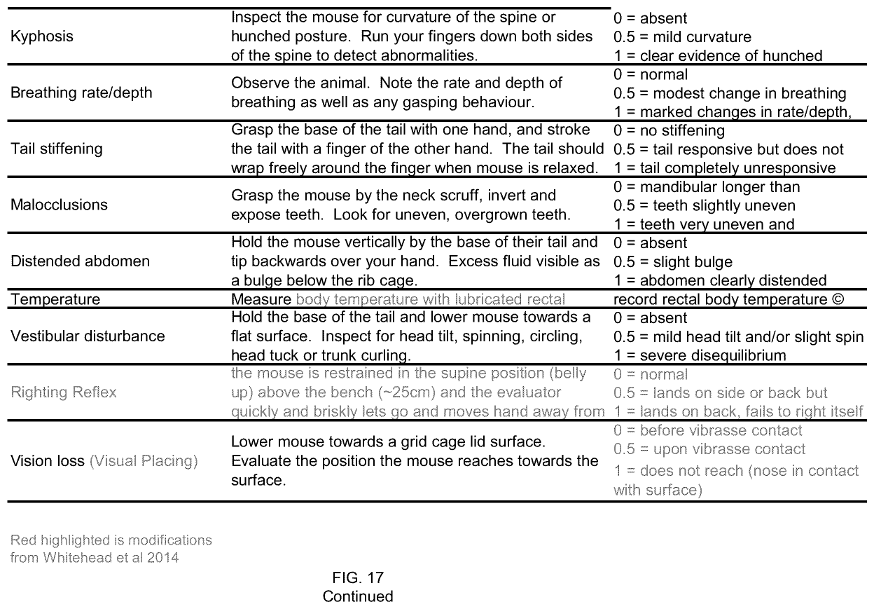

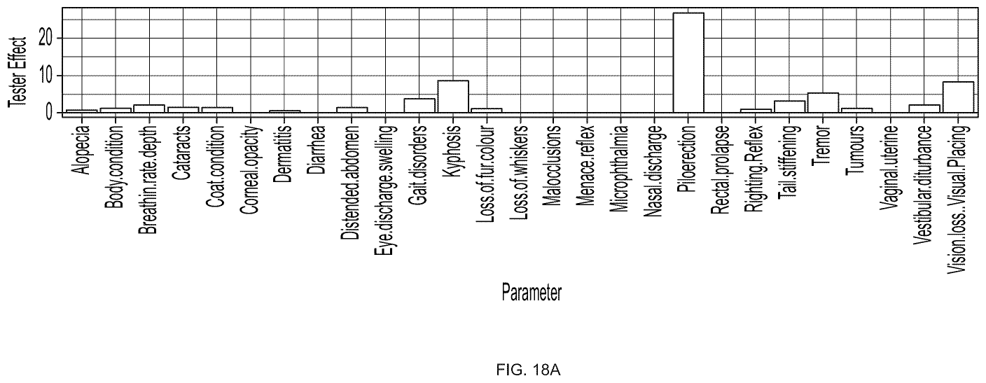

The open field behavioral assays were conducted as previously described [Kumar, V. et al., PNAS 108, 15557-15564 (2011); Geuther, B. et al., Commun. Biol. 2, 124 (2019); Beane, G et al. Video based phenotyping platform for the laboratory mouse. bioRxiv (2022)]. Mice were shipped from Nathan Shock Center aging colony which resides in a different room in the same animal facility at The Jackson Laboratory. The aged mice acclimated for one week to the animal holding room, adjacent to the behavioral testing room. During the day of the open field test, mice were allowed to acclimate to the behavior testing room for 30-45 minutes before the start of the test. One-hour open field testing was performed as previously described. After open field testing, mice were returned to the Nathan Shock Center for manual frailty indexing. Manual frailty indexing was performed within one week of the open field assay; the frailty indexing procedure was modified from Whitehead et al. [Whitehead, J. C. et al., J Gerontol. A Biol. Sci. Med. Sci. 69, 621-632 (2014)]. FIG. 17 shows an FI testing sheet listing all items for manual frailty indexing.

### Video, Segmentation, and Tracking

The open field arena, video apparatus, and tracking and segmentation networks were as described previously [Geuther, B. et al., Commun. Biol. 2, 124 (2019); Beane, G et al. Video based phenotyping platform for the laboratory mouse. bioRxiv (2022)]. The open field arena measured 20.5 inches by 20.5 inches with a Sentech (Omron Sentech, Kanagawa, Japan camera mounted 40 inches above. The camera collected data at 30 frames per second (fps) with a 640×480 pixel (px) resolution. A neural network trained to produce a segmentation mask of the mouse to produce an ellipse fit of the mouse at each frame as well as a mouse track was used.

### Pose Estimation and Gait

Twelve-point 2D pose estimation produced using a deep convolutional neural network trained as previously described [(Sheppard, K. bioRxiv. doi.org/10.1101/2020.12.29.424780 (2020)]. The points captured were nose, left ear, right ear, base of neck, left forepaw, right forepaw, mid spine, left rear paw, right rear paw, base of tail, mid tail and tip of tail. Each point at each frame had an x coordinate, a y coordinate, and a confidence score. A minimum confidence score of 0.3 was used to determine which points were included in the analysis.

Gait metrics were produced as previously described [Sheppard, K. et al., bioRxiv. doi.org/10.1101/2020.12.29.424780 (2020)]. Stride cycles were defined by starting and ending with the left hind paw strike, tracked by the pose estimation. These strides were then analyzed for several temporal, spatial, and whole-body coordination characteristics, producing the gait metrics over the entire video.

### Open Field Measures and Feature Engineering

Open field measures were derived from ellipse tracking of mice as described before [Geuther, B. et al., Commun. Biol. 2, 124 (2019); Geuther B. Q. et al. Elife 10 (2021); Beane, G et al. Video based phenotyping platform for the laboratory mouse. bioRxiv (2022)]. The tracking was used to produce locomotor activity and anxiety features. Grooming was classified using an action detection network as previously described. The other engineered features (spinal mobility, body measurements, and rearing) were all derived using the pose estimation data. The spinal mobility metrics used three points from the pose: the base of the head (A), the middle of the back (B) and the base of the tail (C). For each frame, the distance between A and C (dAC), the distance between point B and the midpoint of line AC (dB), and the angle formed by the points A, B, and C (aABC) were measured. The means, medians, maximum values, minimum values, and standard deviations of dAC, dB, and aABC were taken over all frames and over frames that were not gait frames (where the animal was not walking). For morphometric measures, the distance between the two rear paw points at each frame was measured along with the means, medians, and standard deviations of that distance over all frames.

For rearing, the coordinates of the boundary between the floor and wall of the areana were considered (using OpenCV contour) and added a buffer of four pixels. Whenever the mouse's nose point crossed the buffer, this frame was counted as a rearing frame. Each uninterrupted series of frames where the mouse was rearing (nose crossing the buffer) was counted as a rearing bout. The total number of bouts, the average length of the bouts, the number of bouts in the first five-minutes, and the number of bouts within minutes five to ten were calculated.

### Modeling

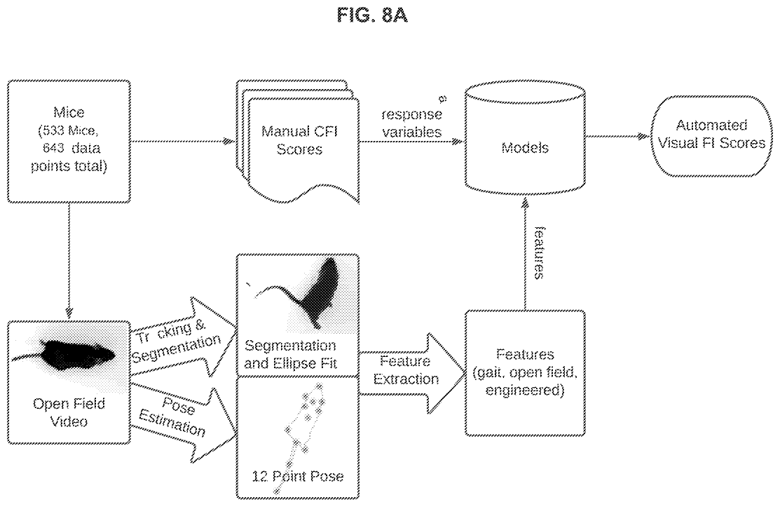

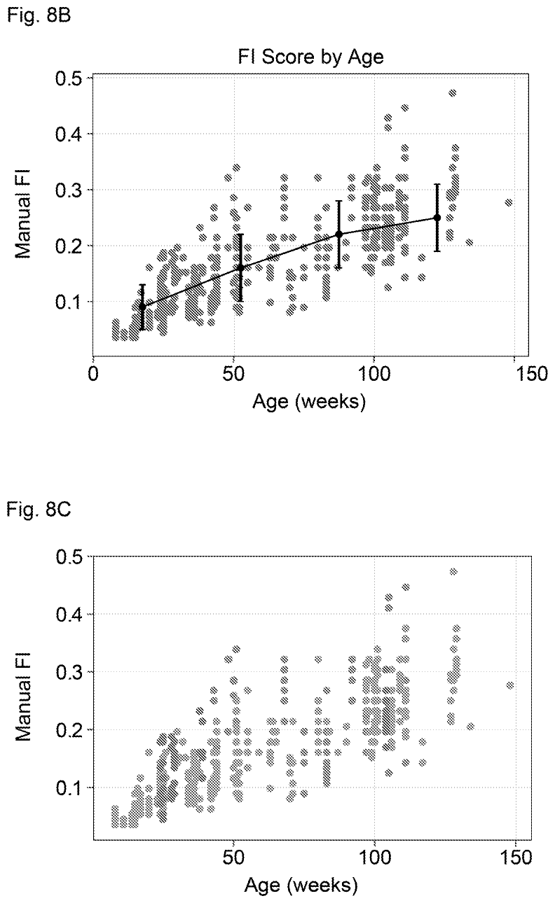

The effect of the scorer was investigated using a linear mixed model with scorer as the random effect and was found that 42% of the variability (RLRT=183.85, p<2:2e⁻¹⁶) in manual FI scores could be accounted for by scorer (FIG. 8C). Restricted likelihood-ratio test (RLRT) [Crainiceanu, C. M & Rupert, D. Journal of the Royal Statistical Society: Series B (2004)] provided strong evidence of scorer (random) effect with non-zero variance. A cumulative link model (logit link) [Agresti, A. Categorical data analysis (2003)] was fit to the ordinal response (frailty parameter) with weight, age, and sex as fixed effects and the tester as a random effect. The effects are estimated variances associated with the random tester effect in the model (Y-axis) across each FI item.

The tester effect was removed from the FI scores using a linear mixed model (LMM) with lme4 R package [Bates, D. et al., JStat Softw 67, 1-48 (2015)]. The following model was fit:

$${y}_{i j} = {μ}_{i} + {ε}_{i j} , {ε}_{i j} ∼ N ( 0 , {σ}^{2} ) , {μ}_{i} ∼ P ≡ N ( 0 , {τ}^{2} )$$

where y_{i,j} was the jth animal scored by tester i, μᵢ was a tester-specific mean, ε_{i,j} was the animal-specific residual, σ² was the within-tester variance and P was the distribution of tester-specific means. Four testers were used, with a different number of animals tested by each tester, i.e., i=1, . . . , 4. The tester effects, estimated with the best linear unbiased predictors (BLUPs) using restricted maximum likelihood estimates [Kenward, M. G. & Roger, J. H. Biometrics, 983-997 (1997)] were subtracted from the FI scores of the animals, \tilde{y}ᵢⱼ=yᵢⱼ−\hat{μ}ᵢ.

Tester-adjusted FI scores, y̅ᵢⱼ, were modeled with video-generated features as covariates/inputs using linear regression model with elastic-net penalty [Zou, H. and Hastie, T., J R. Stat. Soc. Series B Stat. Methodol. 67, 301-320 (2005)], support vector machine [Cortes, C. and Vapnik, V., Mach. Learn. 20, 273-297 (1995)], random forest [Breiman, L., Mach. Learn. 45, 5-32 (2001)], and gradient boosting machine [Friedman, J. H., Ann. Stat. 1189-1232 (2001)]. The data were split randomly into two parts: train (80%) and test (20%). The training data were used to estimate and tune the models' hyper-parameters using 10-fold cross-validation; the test set served as an independent evaluation sample for the models' predictive performance. Fifty different splits were on the data to allow for a proper assessment of uncertainty in the test set results. The models were compared in terms of median absolute error (MAE), root-mean-squared-error (RMSE), and R². These metrics were compared across the four models using repeated-measures ANOVA through F test with Satterthwaite approximation [Fai, A. and Cornelius, P., J Stat. Comput. Sim. 54, 363-378 (1996)] applied to the test statistic's denominator degrees of freedom.

For FRIGHT modeling to predict age with manual FI items, frailty parameters with a single value were removed to avoid unstable model fits, i.e., zero-variance predictors. The ordinal regression models [McCullagh, P. Journal of the Royal Statistical Society: Series B (1980)] were fit without any regularization term and used a global likelihood ratio test (p<2.2e⁻¹⁶) to determine whether the video features show any evidence of predicting each frailty parameter separately, i.e., evidence of a predictive signal. Next, the ordinal regression model was used with an elastic net penalty [Zou, H & Hastie, T, Journal of the Royal Statistical Society: Series B (2005)] to predict frailty parameters using video features.

For predicting manual FI items, frailty parameters were select for which pᵢ<0.80, where i is the mode of the parameters' count distribution. For example, Menace reflex is excluded, since i=1 is the mode for Menace reflex's count distribution with p₁>0.95.

The 100(1−α)% out-of-bag prediction intervals Iₐ (X, Cₙ), where X was the vector of covariates and Cₙ was the training set were obtained via quantile random forests [Meinshausen, N, J Mach. Learn. Res. 7, 983-999 (2006)] with the grf package [Athey, S. et al., Ann. Stat. 47, 1148-1178 (2019)]. Prediction intervals produced with quantile regression forests often perform well in terms of conditional coverage at or above nominal levels i.e. [symbol] [\tilde{y}∈I_{α}(X,Cₙ)|X=x]>1−α where α was set α=0.05.

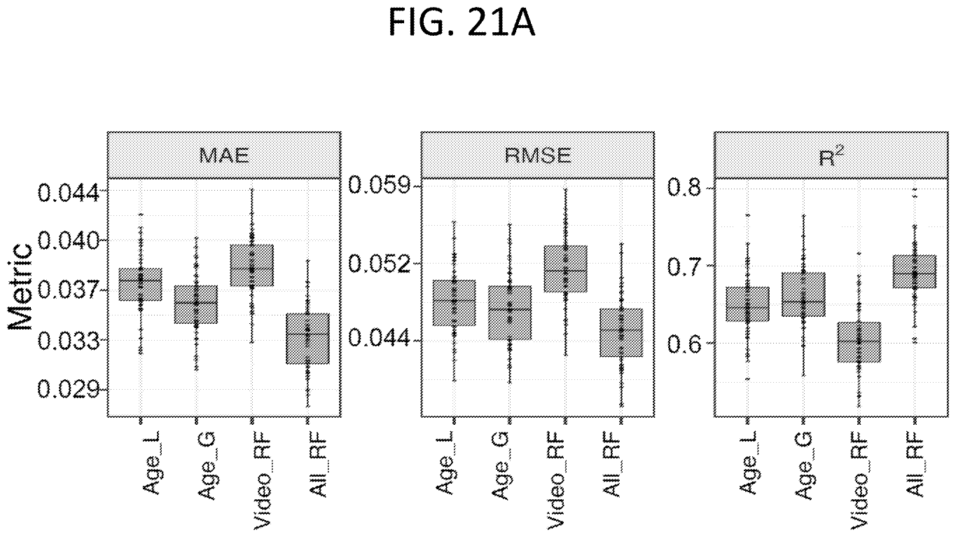

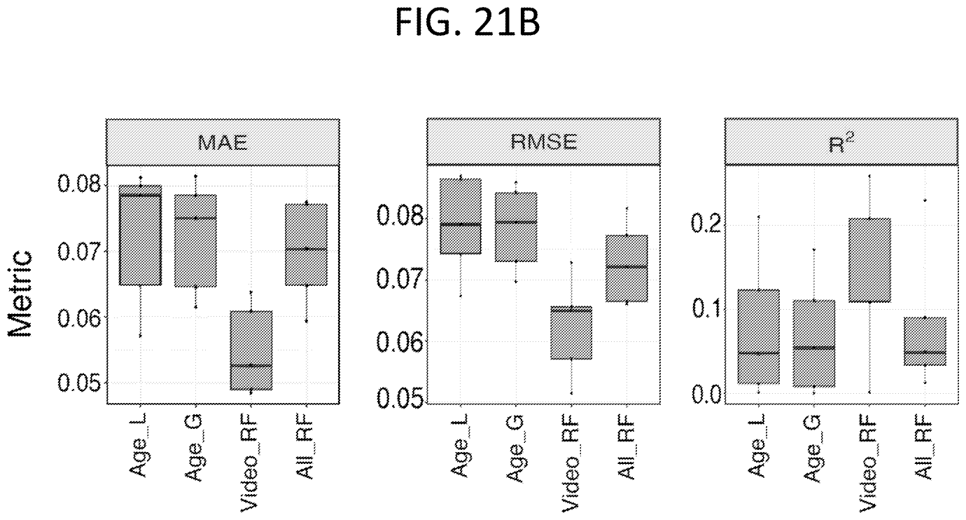

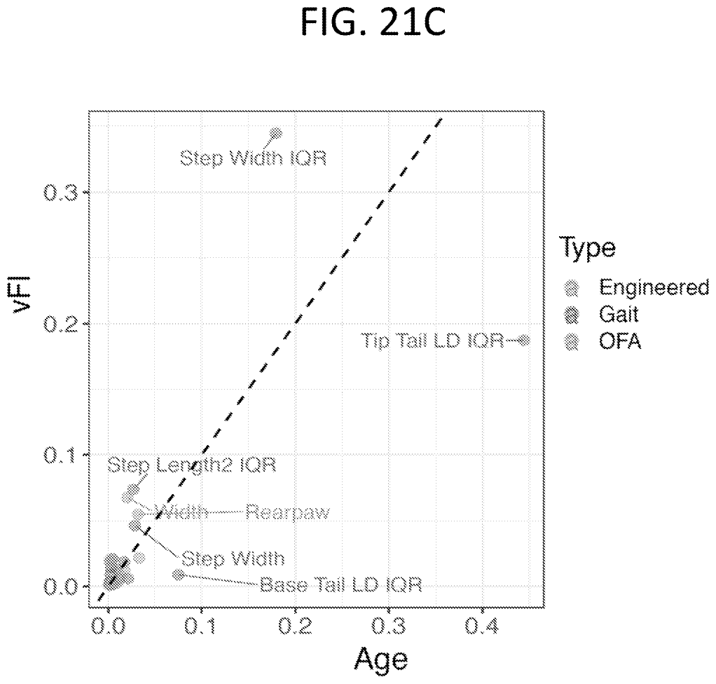

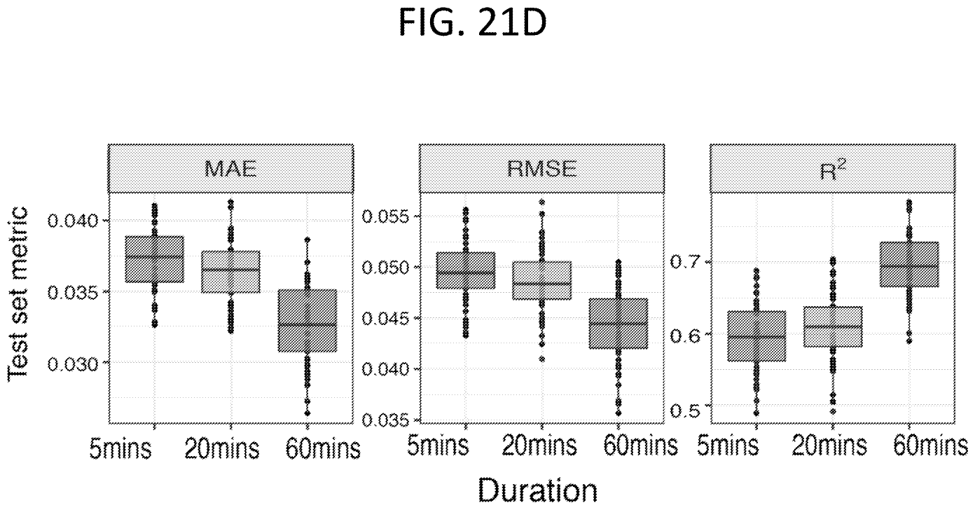

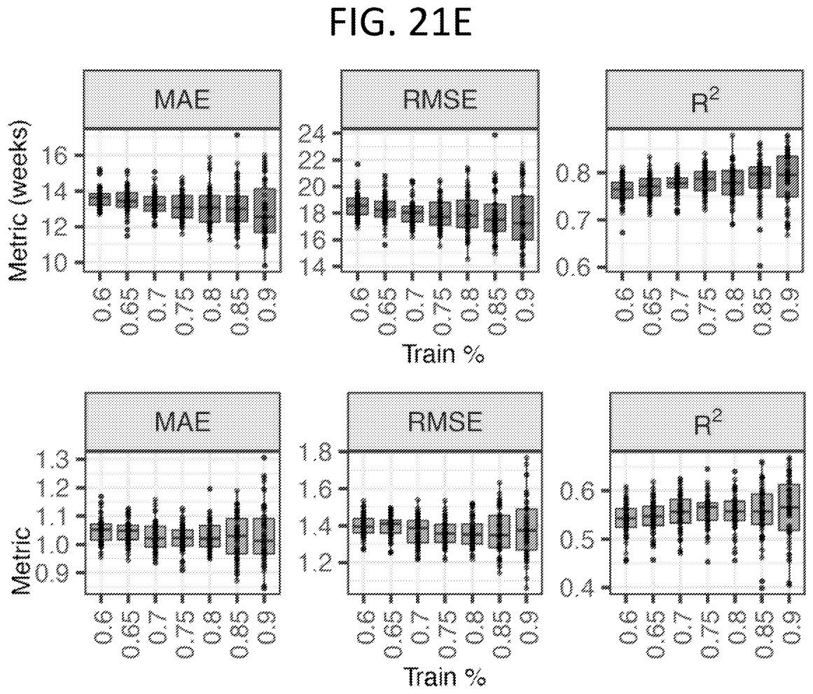

Animals whose ages and FI scores had an inverse relationship were picked, i.e., younger animals with higher FI scores and older animals with lower FI scores. Five test sets were formed containing animals with these criteria and trained the random forest (RF) model on the remaining mice. The predictive accuracy was evaluated for predicting FI scores for the five test sets and the results were displayed (FIG. 21B). The test sets were defined using age_{L}, age_{U}, FI_{L}, and FI_{U}, which denote age and FI cutoffs for young and old animals, respectively. For the five test sets, the parameters were set as follows,

$${age}_{L} = 60 , {age}_{U} = 90 , {FI}_{L} = 0.2 , and {FI}_{U} = 0.15 ,$$

$${age}_{L} = 60 , {age}_{U} = 100 , {FI}_{L} = 0.2 , and {FI}_{U} = 0.15$$

$${age}_{L} = 50 , {age}_{U} = 90 , {FI}_{L} = 0.2 , and {FI}_{U} = 0.2 ,$$

$${age}_{L} = 60 , {age}_{U} = 110 , {FI}_{L} = 0.2 , and {FI}_{U} = 0.2 ,$$

$${age}_{L} = 70 , {age}_{U} = 100 , {FI}_{L} = 0.25 , and {FI}_{U} = 0.15$$

### Data and Code Availability

Code and models were made available at github.com/KumarLabJax and www.kumarlab.org/data. The markdown file in the Github repository github.com/KumarLabJax/vFI-modeling contains details for reproducing results in the manuscript and training models for vFI/Age prediction. The manual FI scores and vFI features for all mice in the dataset can be found there as well. Code for engineered features were made available on github.com/KumarLabJax/vFI-features.

### Results

### Data Collection

The study design as outlined in FIG. 8A evaluated 451 individual C57BL/6J mice (256 males, 195 females), with 117 mice repeated in a second round of testing 5 months later, resulting in a data set of 568 mice ranging from 8 to 148 weeks of age. Top-down video of each mouse in a one hour open field was collected according to previously published protocols [Kumar, V. et al., PNAS 108, 15557-15564 (2011); Geuther, B. et al., Commun. Biol. 2, 124 (2019)] as described in the Methods above herein (FIG. 8A). Following the one hour open field, each mouse was given a standard mouse frailty indexing by a trained expert from the Nathan Shock Center for Aging to assign a manual FI score. Over the course of the data collection, four different scorers conducted the manual FI. The open field video was processed by a tracking network and a pose estimation network, to produce a track, an ellipse-fit, and a 12-point pose of the mouse for each frame [Geuther, B. et al., Commun. Biol. 2, 124 (2019); Sheppard, K. et al., bioRxiv. doi.org/10.1101/2020.12.29.424780 (2020)]. These frame-by-frame measurements were used to calculate a variety of per-video features (all extracted per-video features are listed and defined, with measurement sources, in Table 1), including traditional open field measures such as anxiety, hyperactivity [Geuther, B. et al., Commun. Biol. 2, 124 (2019)], neural network based grooming [Geuther, B. et al., bioRxiv doi.org/10.1101/2020.10.08.331017 (2020)], and novel gait measures [Sheppard, K. et al., bioRxiv. doi.org/10.1101/2020.12.29.424780 (2020)]. The per-video features for each mouse were used as features in an array of machine learning models, including penalized linear regression (LR*) [Zou, H. and Hastie, T., J. R. Stat. Soc. Series B Stat. Methodol. 67, 301-320 (2005)], random forest (RF) [Breiman, L., Mach. Learn. 45, 5-32 (2001)], support vector machine (SVM) [Cortes, C. and Vapnik, V., Mach. Learn. 20, 273-297 (1995)], and extreme gradient boosting (XGB) [Friedman, J. H., Ann. Stat. 1189-1232 (2001)]. The manual FI scores were used as the response variables for the models. As expected, mean FI score increased with increasing age (FIG. 8B). Heterogeneity of FI scores (shown by the standard deviation bars) also increased with age. A sub-maximal limit of FI score slightly below 0.5 was found for the data obtained in this study, which falls within a range of submaximal limits previously shown in mice [Whitehead, J. C. et al., J Gerontol. A Biol. Sci. Med. Sci. 69, 621-632 (2014); Rockwood, K. et al., Sci. Reports. 7, 43068 (2017)]. These results showed that the FI data obtained in this study were typical of other mouse data and mirrored the characteristics of human FIs with the increase in average FI scores and heterogeneity of FI scores with age [Rockwood, K. et al., Sci. Reports. 7, 43068 (2017)]. Visual inspection of scorers indicated that there may have been a scorer-dependent effect on the manual FL. For instance, Scorers 1 and 2 tended to generate high and low frailty scores, respectively. The effect of scorers was investigated using a linear mixed model with scorer as the random effect and found 35% of the variability (RLRT=66.41, p<2.2e⁻¹⁶) in the data set could be accounted for by scorer (FIG. 8C). Restricted likelihood-ratio test (RLRT) [Crainiceanu, C. M. and Ruppert, D., J. R. Stat. Soc. Series B Stat. Methodol. 66, 165-185 (2004)] provided strong evidence of scorer (random) effect with non-zero variance, suggesting that variability between scorers is an important source of variability in the data and should be adjusted prior to modeling.

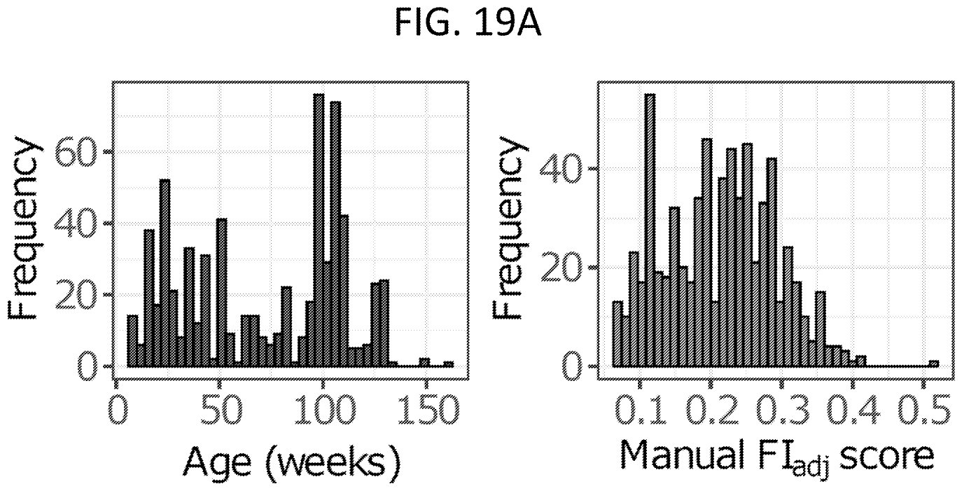

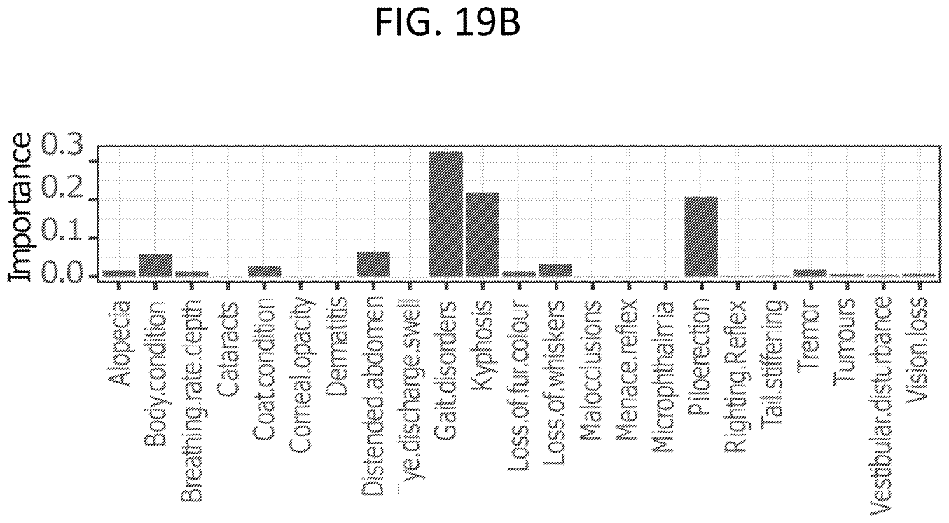

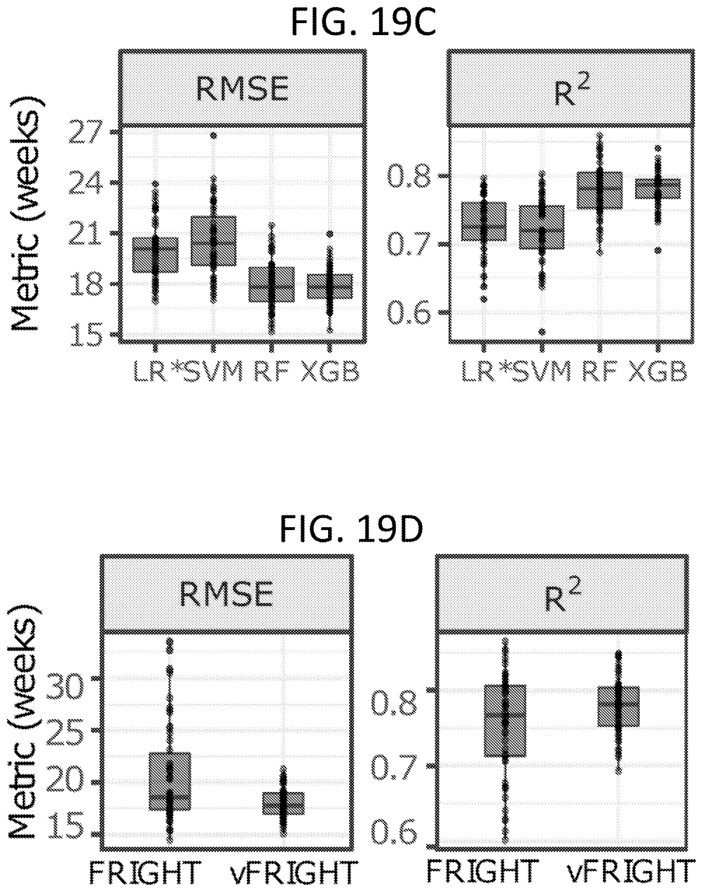

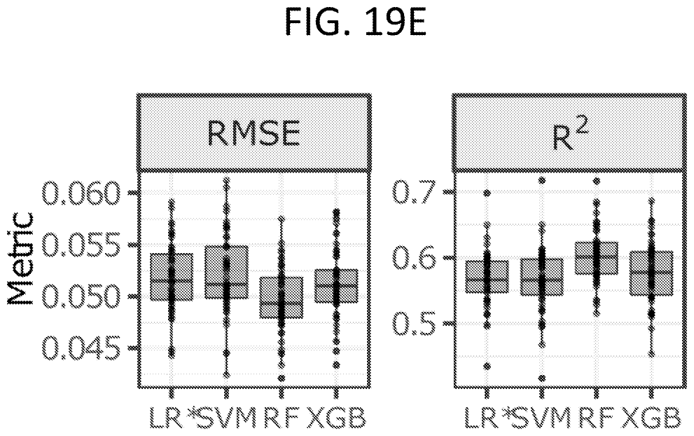

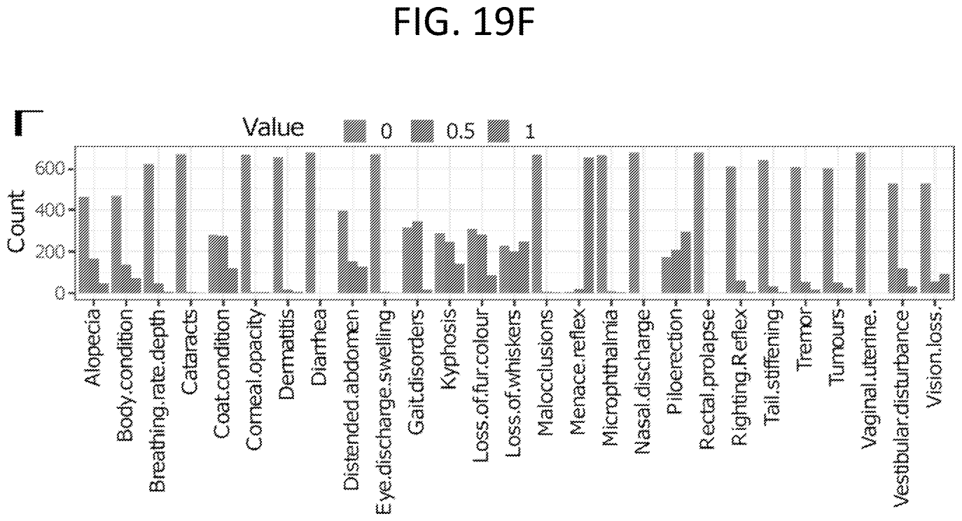

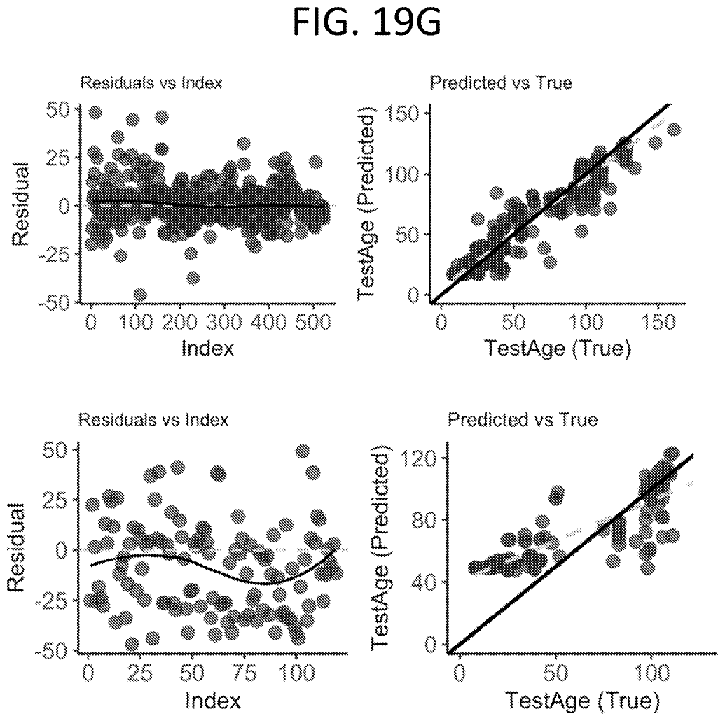

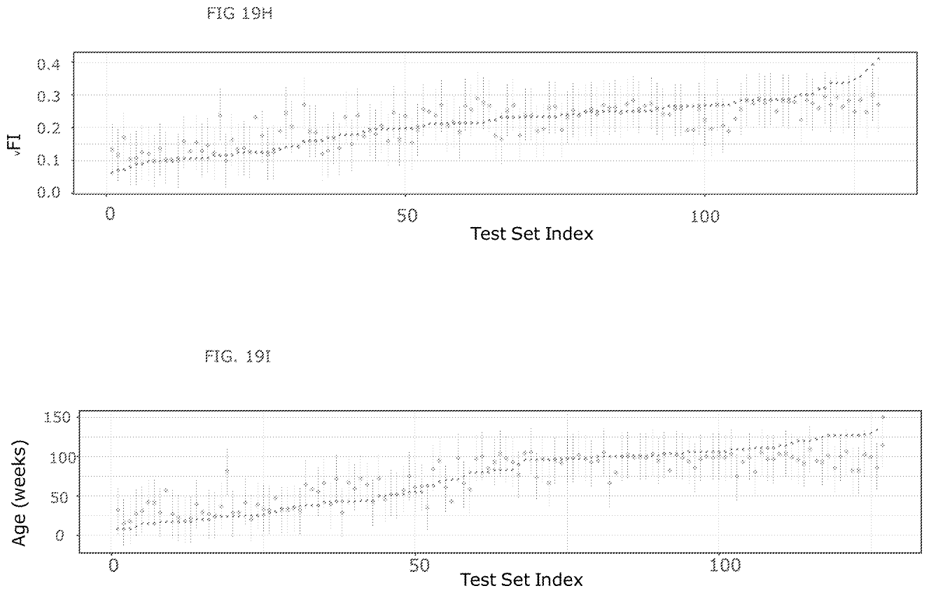

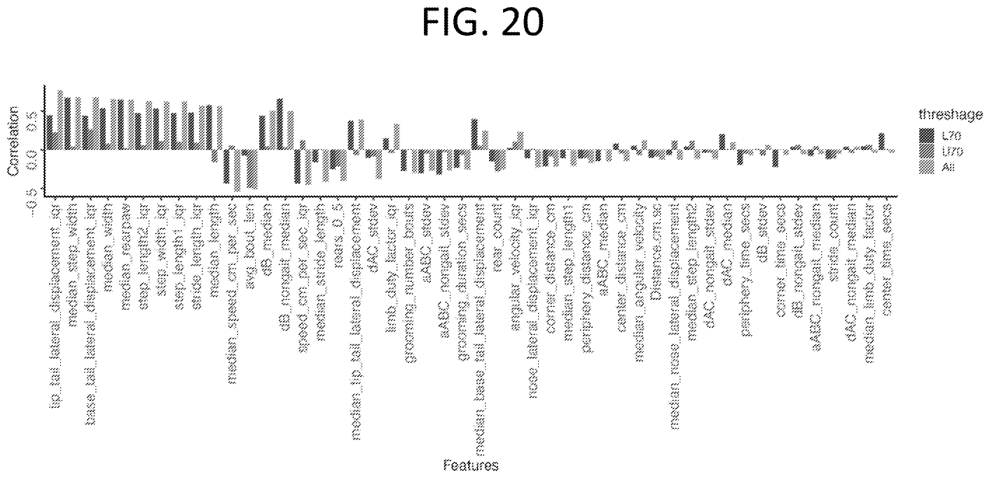

The overall approach is described in FIG. 8A. The study was conducted with 643 data points (371 males, 272 females) taken over three rounds of testing with 533 unique mice. The first round (batch 1) of tests included 222 mice (141 males, 81 females). The second round (batch 2) of tests occurred about five-months later and included 319 mice (173 males, 146 females). Of those mice, 105 were repeated from the first batch. The third round (batch 3) of testing occurred about a year later with 102 mice (57 males, 45 females). Of these mice, 18 had previously been tested in the first round and 15 had been tested in the second round. Top-down video of each mouse in a 1-hour open field session was collected according to previously published protocols [Kumar, V. et al., PNAS 108, 15557-15564 (2011); Geuther, B. et al., Commun. Biol. 2, 124 (2019)] (see Methods and FIG. 8A as examples of a young and old mouse). Following the open field, each mouse was scored using a standard mouse frailty indexing by a trained expert from the Nathan Shock Center for Aging to assign a manual FI score [Sukoff Rizzo, S. J. et al. Current protocols in mouse biology (2018)] (FIG. 19A). Despite bi-modality (Hartigans test [Hartigan, J. A. & Hartigan, P. M., The annals of Statistics (1985)], D=0.07; p<2.2e⁻¹⁶) of the data, it was found that the Simpson's paradox [Simpson, E. H., Journal of the Royal Statistical Society: Series B (1951)] did not manifest in any of the top fifteen features in the data (FIG. 20). The open field video was processed by a tracking network and a pose estimation network, to produce a track, an ellipse-fit, and a twelve-point pose of the mouse for each frame [Geuther, B. et al., Commun. Biol. 2, 124 (2019); Sheppard, K. et al., Cell Reports (2022)]. These frame-by-frame measurements were used to calculate a variety of per-video features, including traditional open field measures of anxiety and hyperactivity [Geuther, B. et al., Commun. Biol. 2, 124 (2019)], grooming [Geuther, B. Q. et al., Elife (2021)], gait and posture measures [Sheppard, K. et al., Cell Reports (2022)], and engineered features. The features used to train a machine learning model to predict chronological and biological age, and a visual FI (vFI) are described herein.

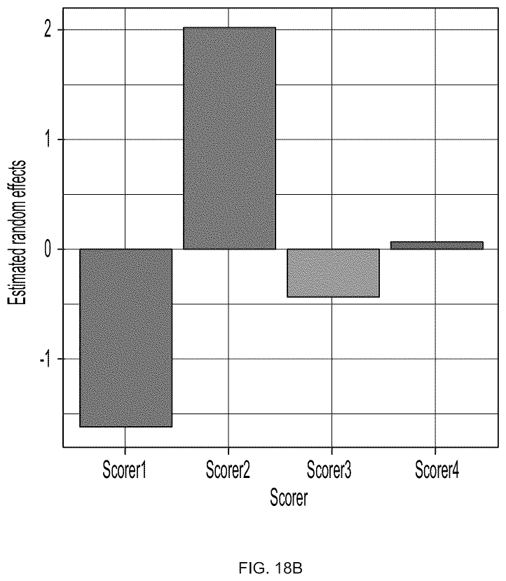

Consistent with previous data, in the dataset, the mean FI score increases with age (FIG. 8B). The heterogeneity of the FI scores (shown by the standard deviation bars) also increases with age. A submaximal limit of the FI score was found slightly below 0.5 for the data, which falls within a range of submaximal limits shown in mice [Whitehead, J. C. et al. Journals of Gerontology Series A (2014); Rockwood, K. et al. Scientific reports (2017)]. These results show that the FI data are typical of other mouse data and mirror the characteristics of human FIs with an increase in average FI scores and heterogeneity of FI scores with age [Rockwood, K. et al. Scientific reports (2017)]. Over the course of the data collection, four different scorers conducted the manual FL. Visual inspection of the data showed a scorer effect on the manual FI score (FIG. 8C). For instance, Scorer 1 and 2 tended to generate high and low frailty scores, respectively (FIG. 18B). The modeling indicated that 42% of the variability in manual FI scores was due to a scorer effect (RLRT=183:85, p<2.2e⁻¹⁶). A closer examination of which FI items are the most affected by the scorer showed that piloerection, kyphosis, vision are the most subjective (FIG. 18A). This analysis suggests that the scorer effect is an important source of variability in mouse clinical FI.

### Feature Extraction

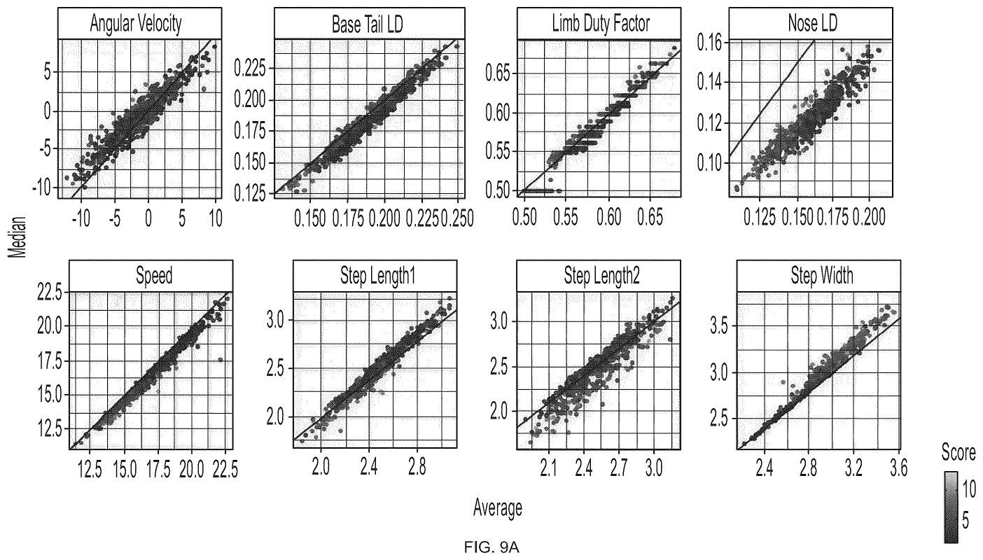

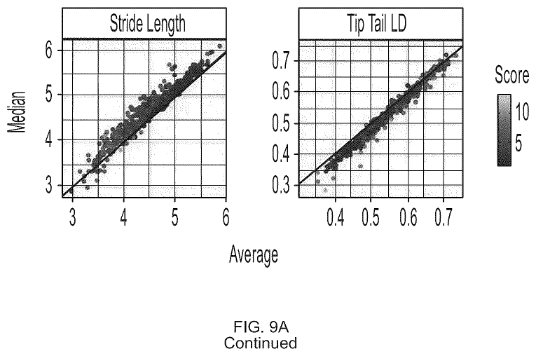

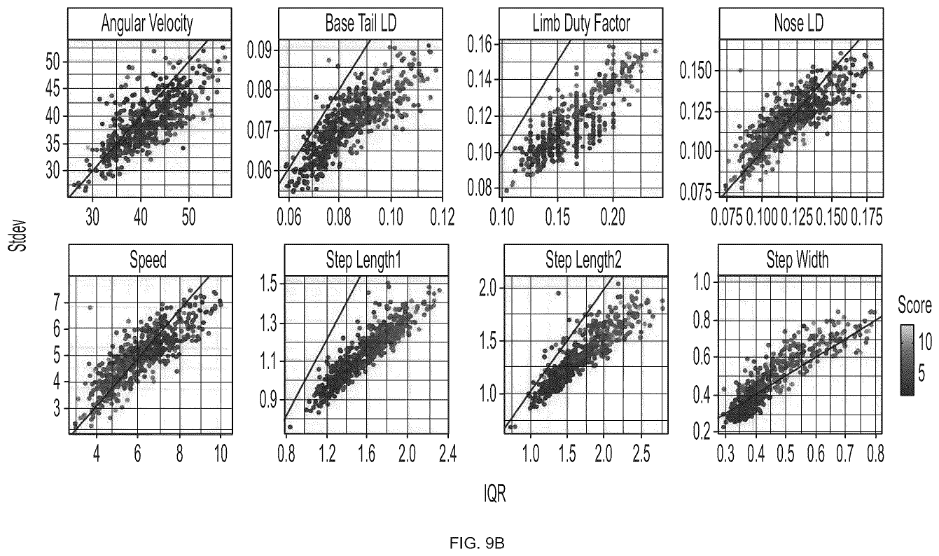

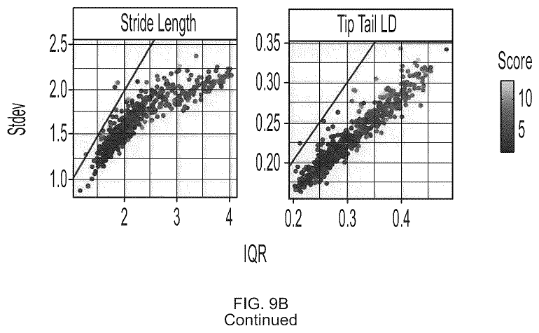

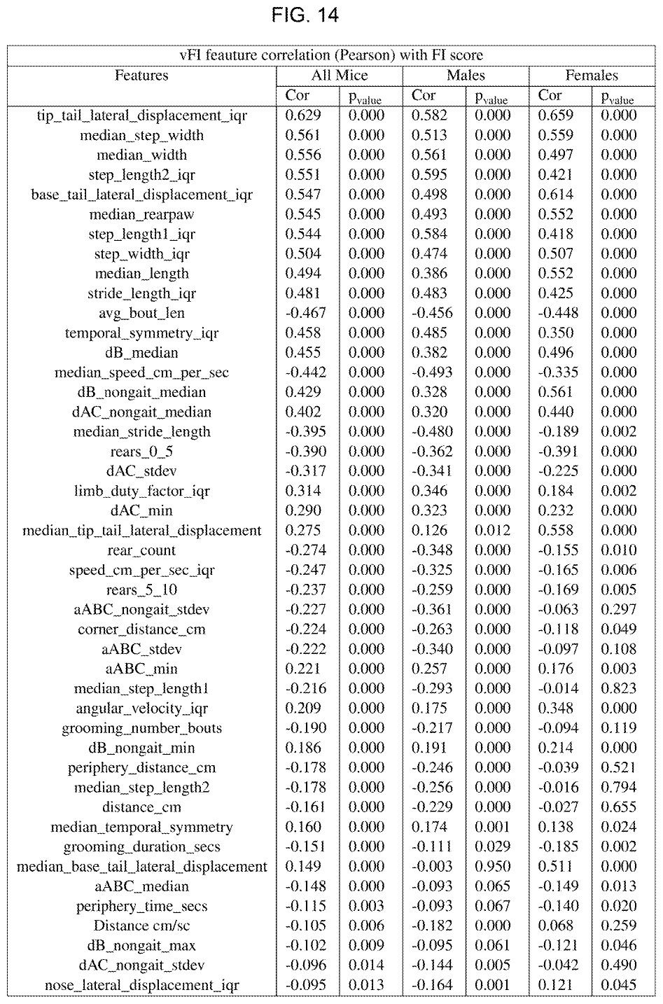

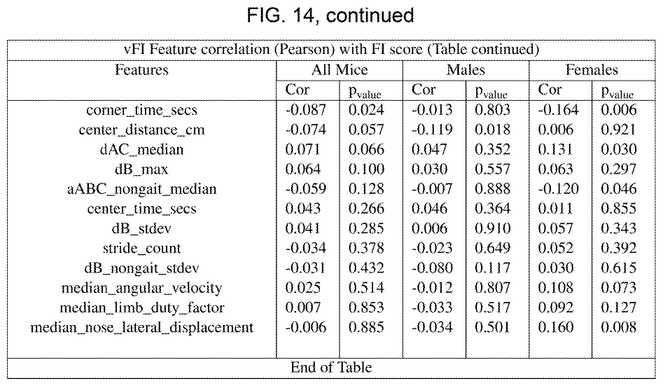

The frame-by-frame segmentation, ellipse fit, and 12-point pose coordinates were used to extract per-video features. Extracted features with explanation and source of the measurements are set forth in Table 1. Overall, there was a very high correlation between median and mean video metrics (FIG. 9A-B). Only medians were used in modeling for two reasons: medians tended to have higher correlation with FI score than means, and medians were more robust to outlier effects than means. Likewise, inter-quartile ranges were used when available and not standard deviations as features in the models, because inter-quartile range tended to be more robust to outliers than standard deviations. This generated a total of 44 video features (FIG. 14, Table 1). Metrics taken in standard open field assays, such as total locomotor activity, time spent in the periphery versus center, and grooming bouts (FIG. 9A) were considered. The standard open-field measures showed low correlation with both FI score and age (FIGS. 14 and 15).

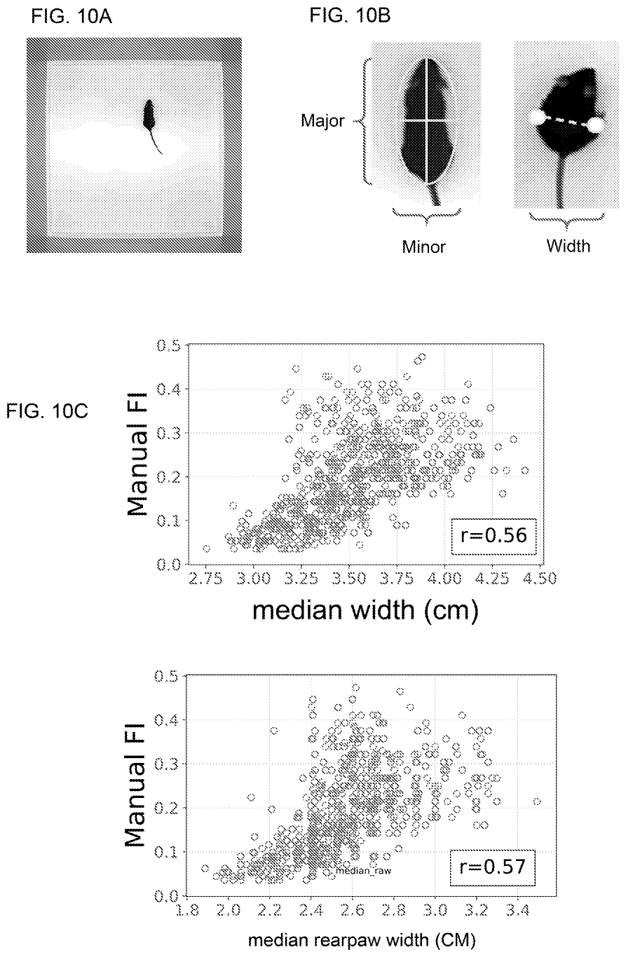

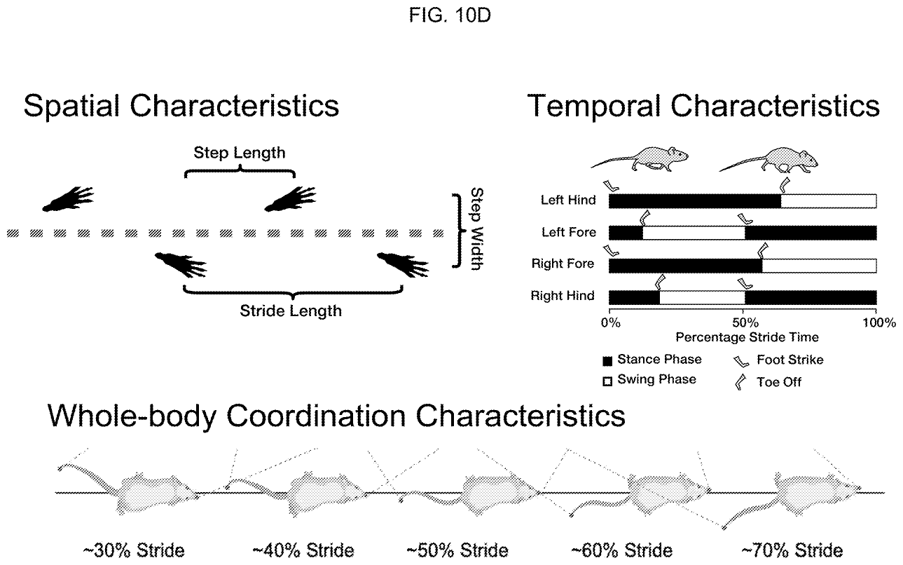

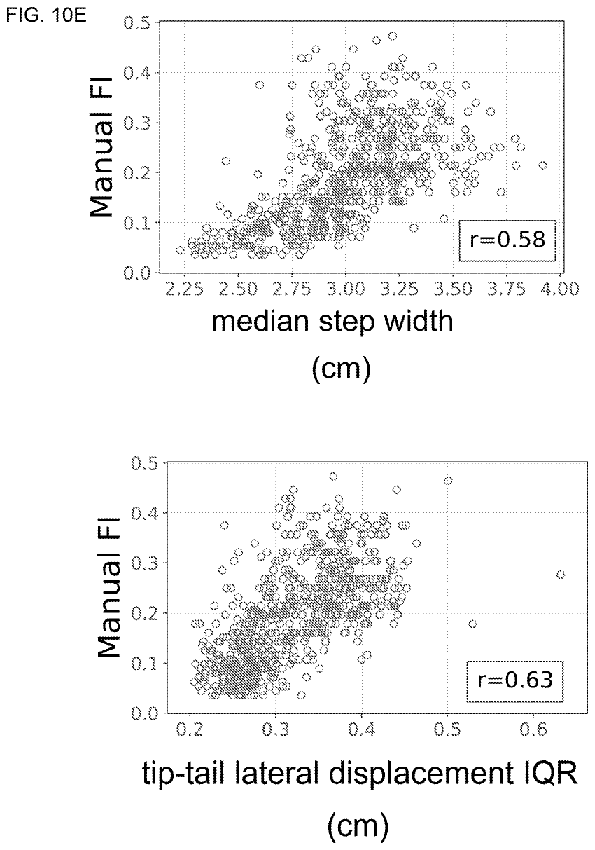

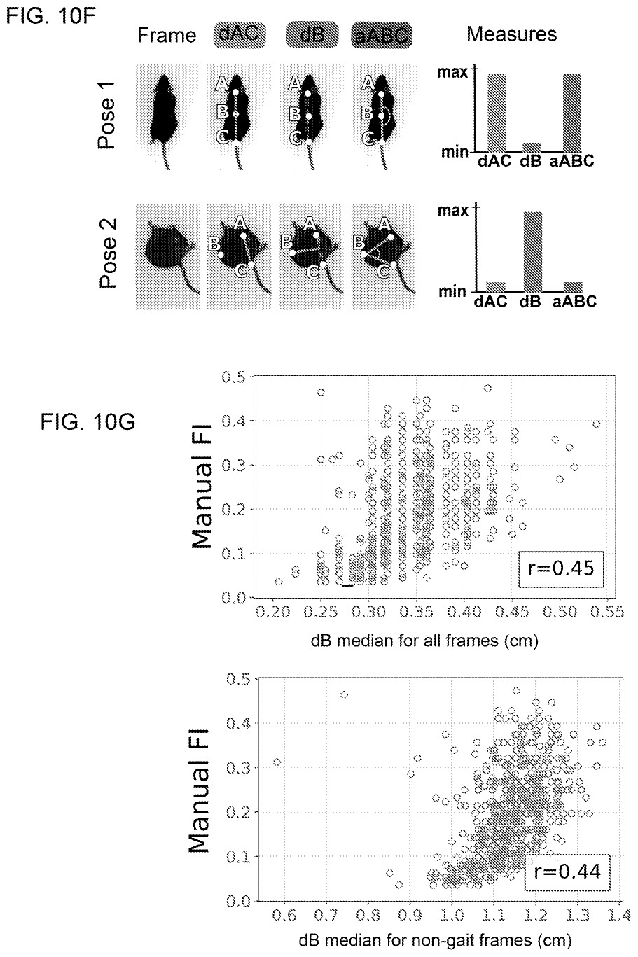

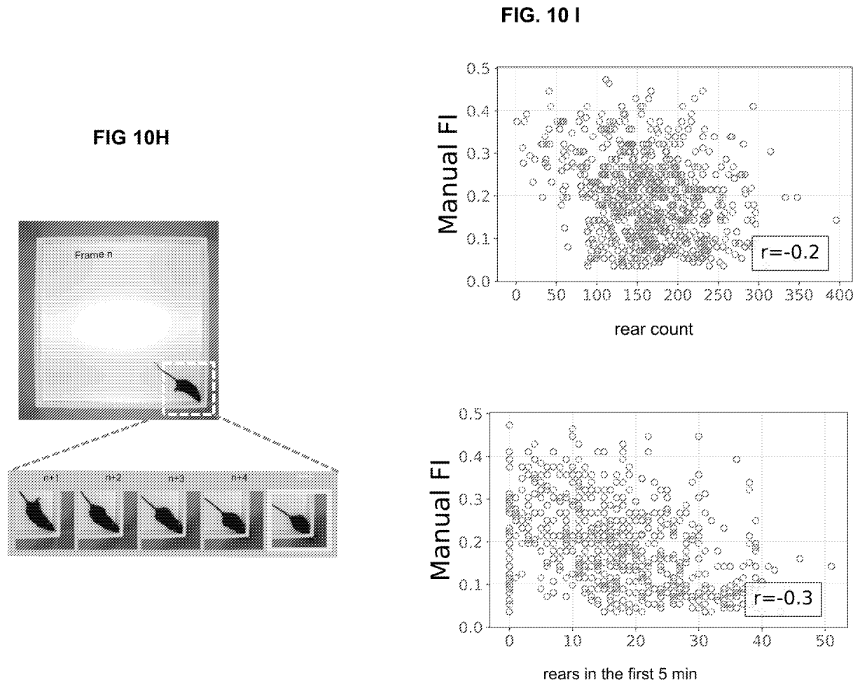

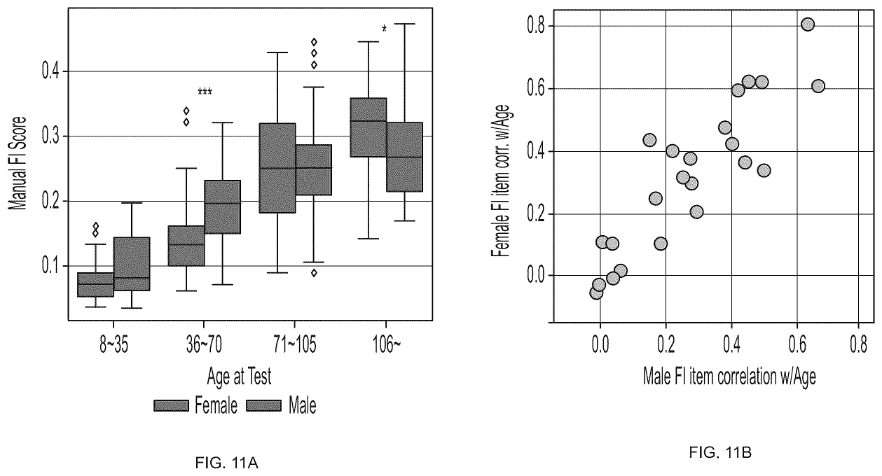

In addition to the existing features, a set of features were designed that were hypothesized to correlate with FL. These features included morphometric features that captured the animals shape and size, as well as behavioral features associated with flexibility and vertical movement. Changes in body composition and fat distribution with age have been observed in humans and rodents [Pappas, L. & Nagy, T. European Journal of Clinical Nutrition 73 (October 2018)]. It was hypothesized that body composition measurements might show some signal of aging and frailty. The major and minor axes of the ellipse fitted to the mouse at each frame were used as an estimated length and width of the mouse respectively (FIG. 10B). The distance between the rear paw coordinates for each frame were taken as another width measurement closer to the hips. The means and medians of the ellipse width, ellipse length, and rear paw width over all frames were used as per-video metrics. Many of these morphometric features showed high correlations with FI score and age (FIGS. 14 and 15), for example, specifically median width and median rear paw width had correlations of r=0.56 and 0.57, respectively (FIG. 10C).

Changes in gait have been shown to be a hallmark of aging in humans [Zhou, Y. et al., Sci. Reports 10, 4426 (2020); Skiadopoulos, A. et al., J. Neuroeng. Rehabil. 17, 41 (2020)] and mice [Tarantini, S. et al., J. Gerontol. A Biol. Sci. Med. Sci. 74, 1417-1421 (2018); Bair, W.-N. et al., J. Gerontol. A Biol. Sci. Med. Sci. 74, 1413-1416 (2019)]. Analyses were performed to explore age-related gait changes in the current cohort of mice (FIG. 10D-E) similar to methods for extracting gait measures from freely moving mice in the open field [Sheppard, K. et al., bioRxiv. doi.org/10.1101/2020.12.29.424780 (2020)]. Each stride was analyzed for its spatial, temporal, and whole-body coordination measures (FIG. 10D), resulting in an array of measures of which the medians over all strides for each mouse were taken. Intra-mouse heterogeneity of gait features was also examined using standard deviations and inter-quartile range over all strides for each mouse. Many of these calculated metrics showed a high correlation with FI score and age (FIGS. 14 and 15), for example, median step width and tip-tail lateral displacement interquartile range (r=0.58 and r=0.63, respectively) (FIG. 1E).

Next, the bend of the spine throughout the video was investigated. It was hypothesized that aged mice bent their spines to a lesser degree, or less often due to reduced flexibility or spine mobility. That change in flexibility could be captured by the pose estimation coordinates of three points on the mouse at each video frame: the back of the head (A), the middle of the back (B), and the base of the tail (C). At each frame, the distance between points A and C normalized for mouse length (dAC), the orthogonal distance of the middle of the back B from the line (dB), and the angle of the three points (aABC) were calculated (FIG. 10F). For each of the three per-frame measures (dAC, dB, and aABC), a mean, median, standard deviation, minimum, and maximum were calculated per video for all frames and for non-gait frames (frames in which the mouse was not in stride). Some moderately high correlations showing relationships between spinal bend and FI score were found which contradicted thathypothesis (FIGS. 14 and 15); while dB median and dAB median (for non-gait frames) were expected to decrease with age, they were found to increase instead (r=0.51 and 0.35, respectively) (FIG. 10G). One possible reason for this result was that very frail mice were spending more time grooming. However, neither grooming bouts nor grooming seconds showed a relationship with FI score or dB median. Another possibility was that high frailty mice walked less and spent more time curled up. That did not seem to be the case either, because there was almost no relationship between either stride count or distance travelled and FI score or dB median. High frailty mice may also have had higher dB medians due to body composition, because dB median had a correlation of 0.496 with body weight. It was also important to note that these bend metrics cast a wide net; they were an inexpensive and general account of all the activity of the spine during the one hour open field. Thus these measures may have captured the interaction between body composition and behavior.

While the previous spinal flexibility measures looked at lateral spinal flexibility, vertical flexibility may also have a relationship to frailty. To investigate this occurrences of rearing supported by the wall were examined (FIG. 10H). It is hypothesized that frailer mice may rear less due to reduced lateral spinal mobility and/or reduced exploratory activity. The edges of the open field were taken and a buffer of five-pixels added as a boundary. The frames in which the nose coordinates of the mouse cross that boundary were used as instances of rearing. From these heuristic rules the number of rears and the average length of each rearing bout was determined (Table 1). Some metrics related to rearing bouts show signal for frailty, specifically total count of rears and rears in the first five-minutes (r=0.2 and 0.3, respectively, FIG. 10I).

Interestingly, the correlations with age were generally slightly higher than FI score (FIGS. 14 and 15), which may have been due to how mice become frail in different ways: one mouse could have high frailty but no dysfunction in its stride width, while on average older mice, regardless of their frailty, may have more stride width changes. Of further note is the increase of heterogeneity in many of these measures with both age and FI score (for example: median width, median step width, dB median).

### Sex

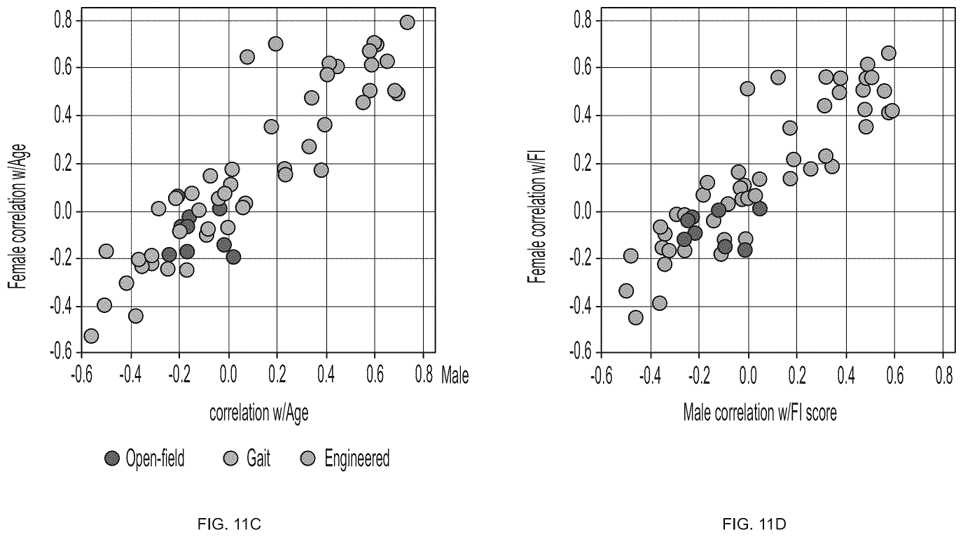

To analyze sex differences in frailty, the FI score data were stratified into four age groups, and the boxplots were compared for each age group between males and females (FIG. 11A). The oldest age group contained only nine females compared to 81 males. The range of females' frailty score for each age group tended to fall slightly lower than the males' except for the oldest age group. The middle two age groups showed highly significant differences in distribution between males and females.

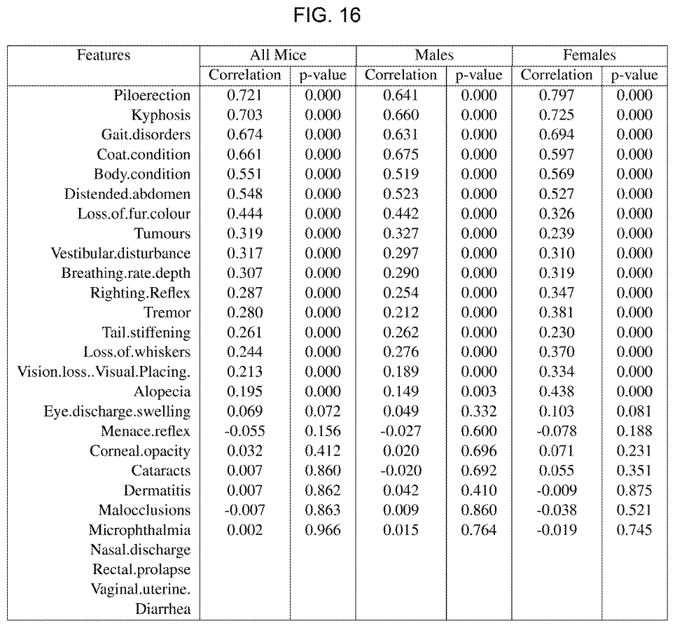

Comparisons between the correlations of male and female FI item scores with age (FIG. 11B), showed an overall high correlation (r=0.85). The average difference between male and female correlations of FI index items with age was 0.08, but there were a few index items showing notable differences. Alopecia and Menace Reflex have the highest sex differences in their correlation to age (by 0.29 and 0.21 respectively) with females having a higher correlation for Alopecia and males having a higher correlation for Menace Reflex (FIG. 16).

The correlations of male and female video features with both FI score and age were also high (r=0.88 and r=0.90 respectively), with an average difference between male and female correlations of video metrics with FI score and age of 0.14 and 0.13 respectively (FIGS. 14 and 15). In both FI score and age, the video features with the highest sex differences were gait measures related to stride and step length, base tail lateral displacement, and tip tail lateral displacement. The highest sex differences were the correlations between median base tail lateral displacement to age (difference of 0.57) and median tip tail lateral displacement to age (0.50), for which females tended to have a higher correlation to both FI score and age. For the metrics related to stride length and step length (median stride length, difference of 0.33), males had a higher correlation to FI score and age than females. These results show that with age, females significantly increase their base-tail and tip-tail lateral displacement in gait while males show little change in this feature, whereas males show a greater reduction in stride length with age compared to females.

Prediction of Age and Frailty Index from Video Data

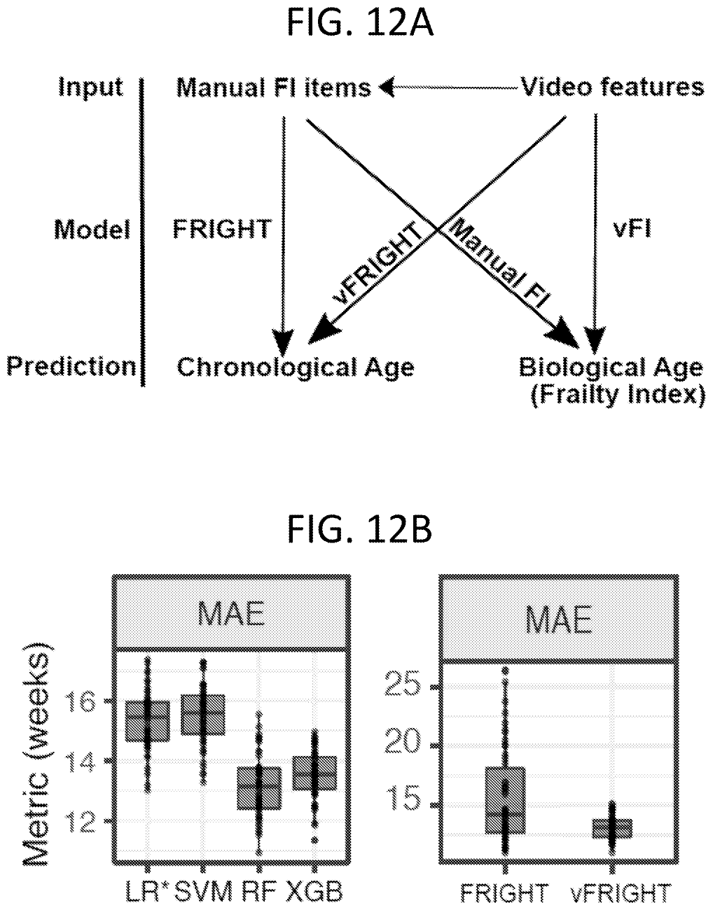

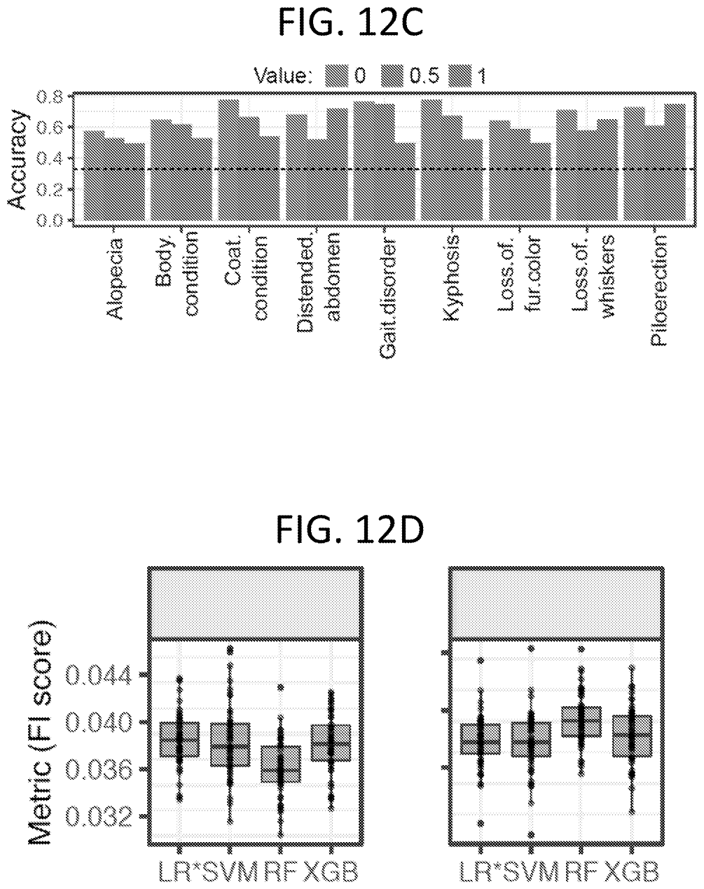

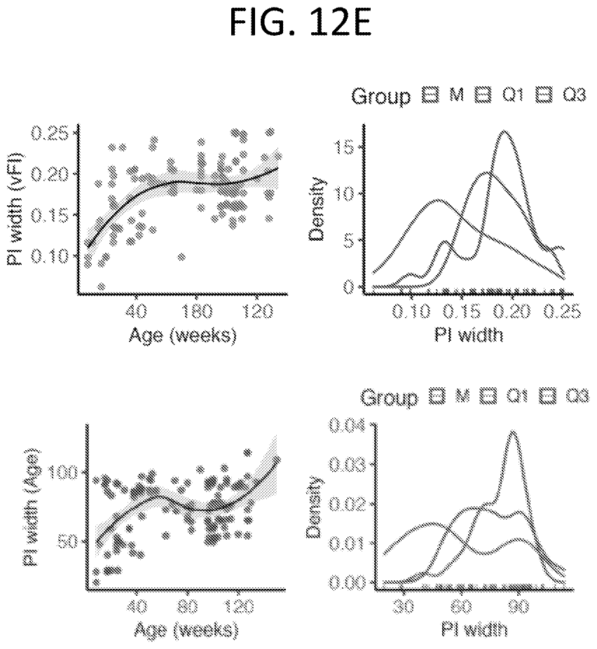

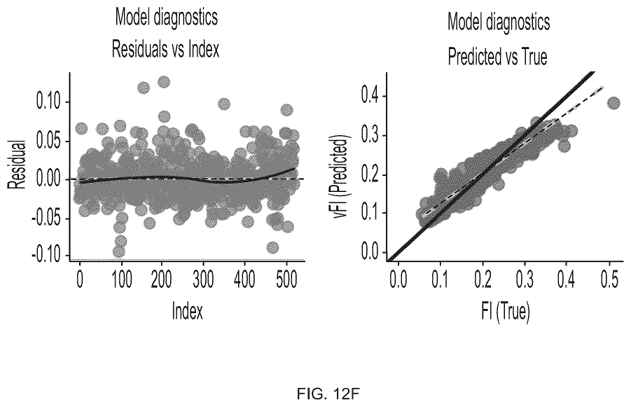

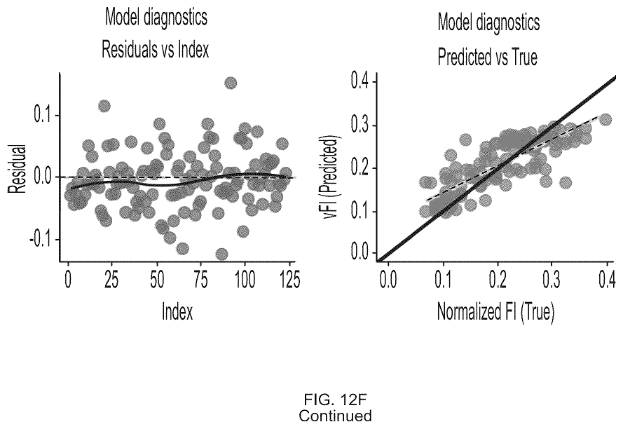

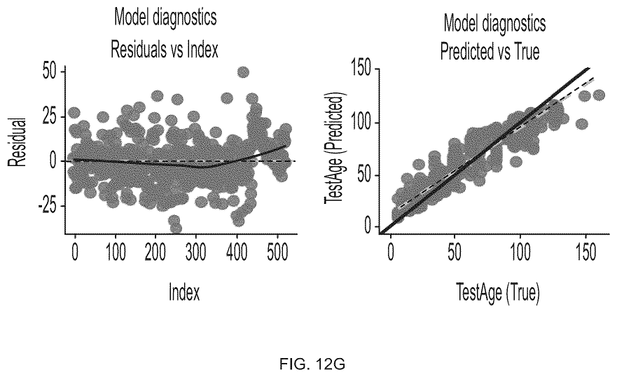

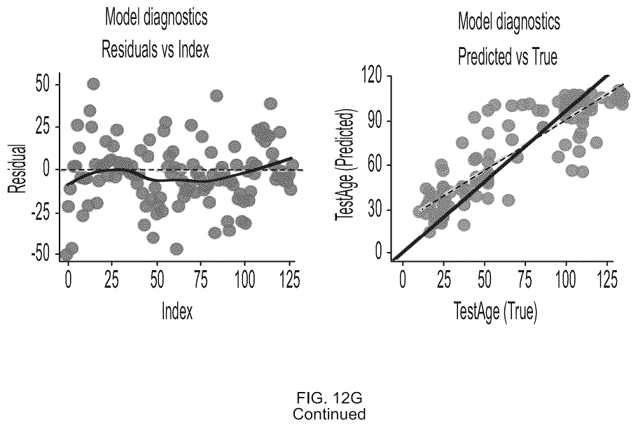

Once it was established that the video features described herein correlate with aging and frailty, these features were used as covariates in a model to predict age and manual FI scores (FIG. 12A), model vFRIGHT and vFI, respectively). Age is an empirical ground truth and has a strong relationship to frailty. The prediction of age using video features (FIG. 12A, Model vFRIGHT) is compared to the prediction of age using manual FI items—a method referred to as the FRIGHT age clock [Schultz, M. B. et al. Nature communications (2020)](FIG. 12A, Model FRIGHT). Four models were tested first-penalized linear regression (LR*) [Zou, H & Hastie, T, Journal of the Royal Statistical Society: Series B (2005)], support vector machine (SVM) [Cortes, C. Vapnik, V. Machine learning (1995)], random forest (RF) [Breiman I., Machine Learning (2001)], and extreme gradient boosting (XGB) [Friedman, J. H, Annals of statistics (2001)] (FIG. 12B, Panel 1). The random forest regression model was selected to predict age on unseen future data due to its superior performance over other models with a lowest mean absolute error (MAE) (p<2.2e⁻¹⁶, F_{3,147}=190.43), root-mean-squared error (RMSE) (p<2.2e⁻¹⁶, F_{3,147}=59.53), and highest R2 (p<2.2e⁻¹⁶, F_{3,147}=58.14) when compared using repeated-measures ANOVA (FIG. 12B, Panel 1, FIG. 19C). The vFRIGHT model was able to more accurately and precisely predict age than the FRIGHT clock. vFRIGHT had a superior performance (p<4.7e⁻⁵, F_{1,49}=19.9, using repeated-measures ANOVA) with a lower MAE (13.1±0.99 weeks) compared to the FRIGHT clock using FI items (15.7±4 weeks) (FIG. 12B, Panel 2). RMSE (RMSEvFRIGHT=17.97±1.44, RMSEFRIGHT=20.62±4.78, p<6.1e⁻⁷, F_{1,49}=32·84) and R2 (RMSEvFRIGHT=0.78±0.04, RMSEFRIGHT=0.76±0.07, p<2.1e⁻⁸, F_{1,49}=44.54) were compared and found similar significant improvements in predicting age when using video features (FIG. 19D). The variance of prediction errors was noticeably reduced for the video based age prediction (vFRIGHT) than the manual FI item based age prediction (FRIGHT) (FIG. 12B, Panel 2). The predicted versus actual values were plotted for the train and test sets for the vFRIGHT model (FIG. 12G) and the FRIGHT model (FIG. 19G). These results combined show that the automated video features more precise information about aging beyond what is addressed in the manual FI items. The video features may also provide information of aging which overlap with the health deficits scored in the manual FI.

To address this, individual FI items using video features were predicted (FIG. 12A). Of the 27 items, many had no to almost non-zero scores, which shows that in the genetically homogeneous data set at least, most of the information in the manual FI are coming from a subset of index items (FIG. 19F). Only index items with a balanced ratio of 0 to 0.5 and 1 scores were selected for prediction (FIG. 12C). A classifier was then built for each of the 9 index items to predict the score given a mouse's video features. The individual FI items' scores were predicted using an ordinal elastic net regression model. For all nine, the score was predicted at an accuracy above what would be expected by randomly guessing (FIG. 12C, dotted line is guessing accuracy). Many of these FI items have implicit relationships to video features like grooming (ex: coat condition, alopecia), gait/mobility (ex: gait disorders, kyphosis), and body composition (ex: distended abdomen, body condition). In the FRIGHT model it was found that gait disorders, kyphosis, and piloerection had the highest contributions to age prediction in our dataset, followed by distended abdomen and body condition (FIG. 19B)—all items that our video features were able to predict the score for (FIG. 12C). These results together showed that most of the information for aging and frailty came from a small subset of manual FI items and that we are able to predict the information in this subset with video data. Furthermore, since age was predicted more accurately and precisely with video data than with manual FI items, video data may also contain additional signals for aging.

Next, the goal of a vFI (FIG. 4A, Model vFI): prediction of manual FI score with video data is addressed. Similarly to the vFRIGHT modeling, the random forest regression model predicted FI score on unseen future data better than all other models, with a lowest mean absolute error (MAE) (p<2·1e⁻¹⁵, F_{3,147}=30.53), root-mean-squared error (RMSE) (p<8:3e⁻¹⁴, F_{3,147}=26.62), and highest R2 (p<4.7e⁻¹⁴, F_{3,147}=27.2) (FIG. 12D, FIG. 19E). The model could predict the FI score within 0.04±0.002 of the actual FI score (FI scores have a possible range of 0 to 1, in the dataset a range of 0.04 to 0.47 was found). This error is akin to 1 FI item mis-scored at 1 or 2 items mis-scored at 0.5 and demonstrates the robustness of the model. The residuals were plotted for the train and test set for the model (FIGS. 12F, 12G, and 19G). The residuals computed from the training data show that their distribution is symmetric around zero for both models, and most residuals fall around the black diagonal line. The residuals for the test set follow similar patterns. It was concluded that the video-generated features described herein can successfully be used for automated frailty scoring. Age has a correlation with manual FI score of r=0.81 which is higher than any video feature. Thus, when we use a model with only age as feature, a higher prediction accuracy is found (FIG. 21A). The model using both video features and age (AllRF) does notably better than the model with age alone, showing that the video features provide important information about frailty (FIG. 21A). When we looked specifically at mice whose FI scores deviated from their age group—younger mice with higher frailty and older mice with lower frailty, the vFI model (VideoRF) performs noticeably better than the models using age and even the model using video features+age (AllRF) (FIG. 21B). This shows that for mice who are outliers of their age group, video features provides better information than age.

Finally, to see how much training data is realistically needed for high performance prediction with vFI and vFRIGHT, a simulation study was performed where different percentage of total data was allocated to training (FIG. 21E). It was found that a training set of <80% of the current dataset achieved similar performance while a decrease in the training set size below this shows a general downward trend in performance. As open field tests are sometimes done with lengths shorter than one-hour, next investigated the decrease in accuracy is investigated for vFI predictions using videos with shorter lengths by truncating videos to the first five and first 20 minutes (FIG. 21D). The features associated with 60-minute videos had the best accuracy for vFI prediction (LMM where ‘simulation’ is the random effect; lowest MAE, F_{2,98}=178.39, p<2.2e⁻¹⁶; lowest RMSE, F_{2,98}=156.93, p<2.2e⁻¹⁶); highest R2 (p<2.2e⁻¹⁶, F_{2,98}=297.3). A significant drop in performance accuracy was observed when the open field test length is reduced from 60 to 20-minute video (LMM with post hoc pairwise comparisons—MAE, t98=14.82, FDR-adjusted p<0:0001; RMSE, t98=13.69, FDR-adjusted p<0:0001; R2, t98=−19:22, FDR-adjusted p<0:0001). Based on experiments, it was concluded that while 60-minute video-generated features provide the most accurate vFI predictions there is not a substantial loss in accuracy in predictions even with 80% of the videos.

### Quantifying Uncertainty in Frailty Index Predictions

In addition to quantifying an average accuracy, error was also investigated more closely within the data set. The prediction error was quantified by providing prediction intervals (PIs) that gave a range of values, containing the unknown age and FI score with a specified level of confidence, based on the same data that gave random forest point predictions [Zhang, H. et al., Am. Stat. 74, 392-406 (2020)]. One approach for obtaining random forest-based prediction intervals involved modeling the conditional distribution of FI given the features using generalized random forests as previously described [Meinshausen, N., J. Mach. Learn. Res. 7, 983-999 (2006); Athey, S. et al., Ann. Stat. 47, 1148-1178 (2019)]. For animals in the test set, generalized random forests based on quantiles were used to provide the point predictions of the FI score (Age resp.) and prediction intervals, which gave a range of FI (Age resp.) values that would contain the unknown FI scores (resp. Age) with 95% confidence (FIG. 12J-I). The average PI width for all test animals' predicted FI score was 5.72±1.49 (resp. 80.29±16.8 for predicted Age), while the PI lengths ranged from 2.3 to 8.5 (resp. 28 to 114 for Age), highlighting that the widths of the PIs were animal and age-group specific. A smoothed regression fit was plotted for PIs' width versus age that indicated the widths increased with the animal's age (FIG. 12G-H). The variability of 95% PI widths, displayed in FIG. 124G-H (right panels), showed higher variability for animals belonging to the middle age groups (“M”, pink). Going beyond simple point predictions, prediction intervals (PIs) of the frailty index were provided to quantify the predictions' uncertainty, allowing the FI score and age to be pinpointed with higher accuracy for some animals than others.

### Feature Importance for Frail and Healthy Animals

A useful visual FI (vFI) should depend on several features that can capture the animal's inherent frailty and be interpretable simultaneously. Two approaches were used to identify features important for making vFI predictions using the trained random forest model: (1) feature importance and (2) feature interaction strengths. Feature importance provided a measure of how often the random forest model used the feature at different depths in the forest. A higher importance value indicated that the feature occurred at the top of the forest and was thus crucial for building the predictive model. For the second approach, a total interaction measure was derived that indicated to what extent a feature interacted with all other model features.

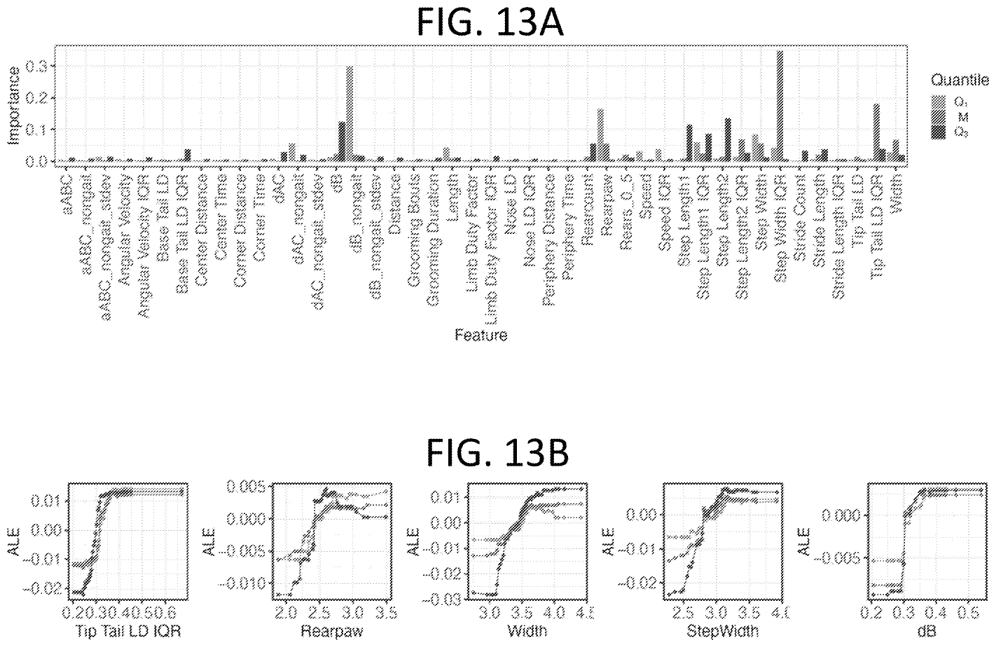

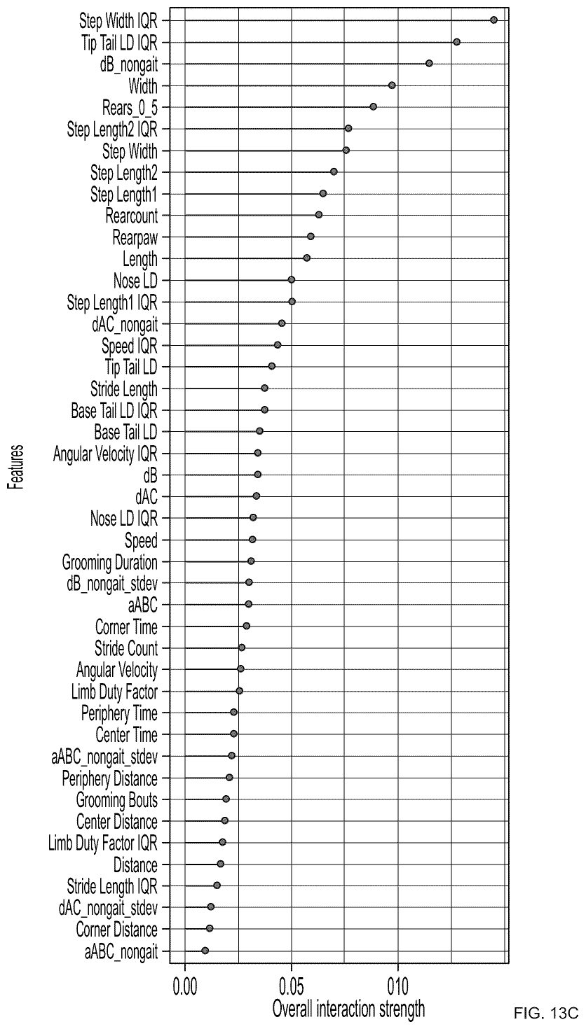

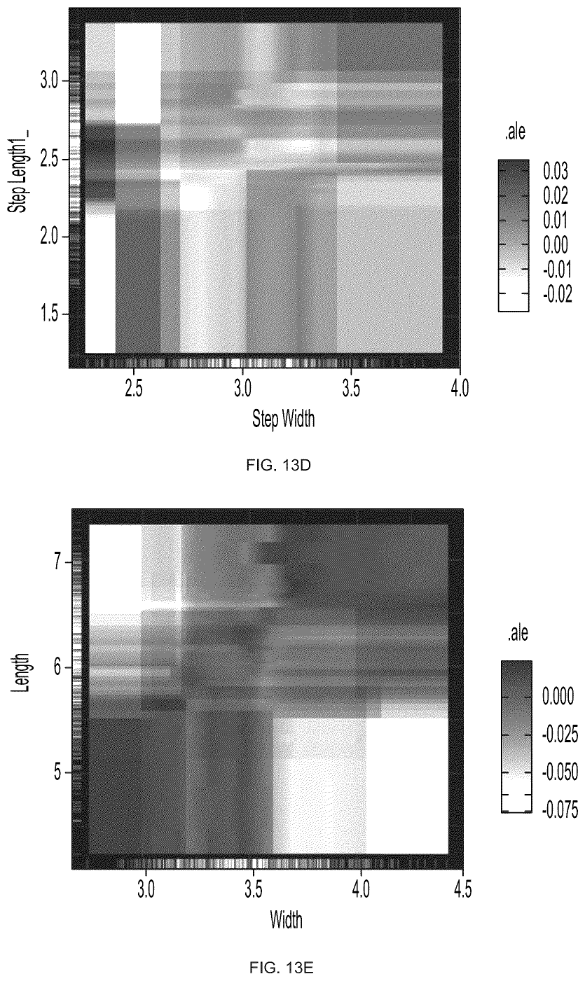

A comparison of the feature importance's for the vFI and vFRIGHT models (FIG. 11A) shows that though many of the most important video features to the model are shared, there are a couple key differences (FIG. 21C). For example, step width IQR is much more important for the vFI than for vFRIGHT, and tip-tail lateral displacement (LD) IQR is much more important for vFRIGHT than for vFI. A more complete picture of the feature importance was obtained by modeling three different quantiles of the conditional distribution of the FI score. The three quantiles represent three frailty groups: low frail (Q1), intermediate frail (M), and high frail (Q3) mice. It was hypothesized that different sets of features are crucial for mice belonging to different frailty groups. Indeed, step length1 IQR was crucial in mice belonging to both Q1 and Q3 quantiles (FIG. 13A). In addition, features such as length, rear paw speed, dAC/dB (non-gait) and step width were important for lower frailty mice, whereas step lengths dB and rear count were more important for animals with high frailty. Similarly, step width, tip tail LD, and width were critical for mice with an FI score close to M.

For the feature interaction strength approach, H-statistic [Friedman, J. H. et al., Ann. Appl. Stat. 2, 916-954 (2008)] was used as the interaction metric that measured the fraction of variability in predictions explained by feature interactions after considering the individual features. For example, 15% of the prediction function variability was explained due to interaction between tip tail LD and other features after considering the individual contributions due to tip tail LD and other features. About 13% and 8% of the prediction function variability was explained due to interaction between width (resp. step length) and other features. For a deeper analysis, all the two-way interactions between tip tail LD and the other features were inspected (results not shown). Strong tip tail LD interactions with width, stride length, rear paw, and dB of the animal were found.

Both feature importance and feature interaction strengths indicated that the trained random forest for vFI depended on several features and their interactions. However, they did not indicate how the vFI depended on these features and how the interactions look. The accumulated local effect (ALE) plots [Apley, D. W. and Zhu, J., J R. Stat. Soc. Series B Stat. Methodol. 82, 1059-1086 (2020)] that described how features influenced the random forest model's vFI predictions on average were used. For example, an increasing tip tail lateral displacement positively impacted (increased) the predicted FI score for animals in intermediate and high frail groups (FIG. 13B). Similarly, an increasing rear paw measure positively affected the predictions—the impact was most visible for animals in the high frail group. Animals with larger widths positively affected predictions; larger step widths and dBs positively impacted model predictions. Thus the ALE plots for important features provided clear interpretations that agreed with the initial hypotheses. The ALE second-order interaction effect plot was explored for the step length1-step width (FIG. 13D) and Length-Width (FIG. 13E) predictors. It revealed the two features' additional interaction effects and did not include the main features' marginal effects. FIG. 13D revealed an interaction between step width and step length: larger step widths and step lengths increased the predicted FI scores. Similarly, larger widths (36-44 cm) and lengths (52-60 cm) positively impacted the average FI scores predictions.

To summarize, vFI's utility was established by demonstrating its dependence on several features through marginal feature importance and feature interactions. Next, the ALE plots were used to understand the effects of features on the model predictions, which helped relate the black-box models' predictions to some of the video-generated features. Opening the black-box model was an essential final step in the modeling framework.

### Discussion

The mouse FI is an invaluable tool in the study of biological aging. The studies described herein sought to extend it by producing an automated visual frailty index (vFI) using video-generated features to model FI score. This vFI offered a reliable high-throughput method for studying aging. One of the largest frailty data sets for the mouse was generated with associated open field video data. Computer vision techniques were used to extract behavioral and morphometric features, many of which showed strong correlations with aging and frailty. Sex-specific aging in mice was also analyzed. Machine learning classifiers were then trained that could accurately predict frailty from video features. Through modeling, insight into feature importance across age and frailty status was also gained.

The data collected at a national aging center with similar design as one would in a high-throughput interventional study that may run for several years. The mice were tested by the trained scorer who was available; four different scorers were used to FI test the different batches of mice. Further, there were some personnel changes between batches. These conditions may provide a more realistic example of inter-lab conditions where discussion and refinement would be difficult. It was found that 42% of the variability in the data set could be accounted for by the scorer, indicating the presence of a tester effect. This variability affected some items, such as piloerection, more than others. Although previous studies looking at tester effect found good to high inter-reliability between testers in most cases, FI items showing lower inter-reliability required discussion and refinement for improvement [Kane, A. E., Ayaz, O., Ghimire, A., Feridooni, H. A. & Howlett, S. E., Canadian journal of physiology and pharmacology (2017)].

Top-down videos of mice in the open field were processed by previously trained neural-networks to produce an ellipse-fit and segmentation of the mouse as well as a pose estimation of 12 salient points on the mouse for each frame. These frame-by-frame measures were used to engineer features to use in the models. The first category of features were standard open field metrics such as time spent in the periphery vs center, total distance travelled, and count of grooming bouts. These standard open field metrics had poor correlation with both FI score and age. These results suggested that standard open field assays are inadequate to study aging.

In humans, changes in age-related body composition and anthropometric measures such as waist-to-hip ratio are predictors of health conditions and mortality risk [Mizrahi-Lehrer, E., Cepeda-Valery, B. & Romero-Corral, Handbook of Anthropometry: Physical Measures of Human Form in Health and Disease (2021); Pappas, L. E. & Tim R, N., European journal of clinical nutrition (2019); Gerbaix, M., Metz, L., Ringot, E. & Courteix, D., Lipids in health and disease (2010)]. The effect of aging on body composition in rodent models is less established, though there are observed changes in body composition similar to humans [Mizrahi-Lehrer, E., Cepeda-Valery, B. & Romero-Corral, Handbook of Anthropometry: Physical Measures of Human Form in Health and Disease (2021); Gerbaix, M., Metz, L., Ringot, E. & Courteix, D., Lipids in health and disease (2010)]. A high correlation is found between morphometric features and both FI score and age, in particular median width and median rear paw width.

The prevalence of gait disorders increase with age [Zhou, Y et al. Scientific Reports (2020)]. Geriatric patients are shown to have gait irregularities; for example, older adults have increased step width variability [Tarantini, S. et al. The Journals of Gerontology: Series A (2018)]. The spatial, temporal, and postural characteristics of gait for each mouse were examined and found many features with a strong correlation with both frailty and age. Analogous to human data, a decrease in stride speed with age was observed, as well as an increase in step width variability [Tarantini, S. et al. The Journals of Gerontology: Series A (2018)]. As gait is thought to have both cognitive and muscular-skeletal components, it is a compelling area for frailty research.

Spinal mobility in humans is a predictor of quality of life in aged populations and the mouse is used as a model for the aging human spine. Surprisingly, though some spinal bend metrics showed moderately high correlations with FI score, the relationship was the opposite of what was initially hypothesized. Because these metrics were a general account of all the activity of the spine during the experiment, they were likely capturing a combination of behaviors and body composition which gave the observed result. Nevertheless, some of these metrics showed a moderately high correlation with FI score and age and were deemed important features in the model.

Many age-related biochemical and physiological changes are known to be sex-specific. Understanding sex differences in the presentation and progression of frailty in mice is crucial for translating pre-clinical results for clinical use. It is of interest to understand how sex characteristics such as hormones and body fat distribution relate to biological aging. In humans, there is a known ‘mortality-morbidity paradox’ or ‘sex-frailty paradox’, in which women tend to be more frail but paradoxically live longer. In C57BL/6J mice, however, it seems males tend to live slightly longer than females, though there is variability, and females do not seem to paradoxically live longer when frail. The study described herein found more males surviving to old age than females, and further found that females tended to have slightly lower frailty distributions than males of the same age group. These results suggested that in mice, the sex-frailty paradox shown in humans may not exist or may be reversed. The correlations of FI index items to age were compared between males and females and some sex differences in the strength of correlation were found for a few of the index items, mostly related to visual fur changes. When comparing the correlations of the video features to age and FI score between males and females, a number of starkly different correlations were also found. Median base tail lateral displacement and median tip tail lateral displacement were both much more strongly correlated with age for females than for males; as female mice age, their tail lateral displacement within a stride tended to increase, while males showed almost no change. On the other hand, males showed a strong decrease in stride length and a strong increase in step length with age, while females showed very little change. Most video features with higher differences were gait-related, with several related to spinal bend. These differences in gait with age were a new insight. Understanding how sex differences in human frailty compare to mouse frailty is important in order to critically evaluate how results from mouse studies could translate to humans.

The manual FI evaluates a wider range of body systems than vFI. However, the complex behaviors measured and described herein contain implicit information about many body systems. In the isogenic dataset, most information in the manual FI came from a limited subset of index items. Of the 27 manual FI items scored, 18 items had little to no variation in score in our dataset (almost all mice had the same score, i.e. 0), and only nine items had a balanced distribution of scores. The video features can accurately predict those nine FI items. The model using video features also predicted age more accurately with much less variance than the model using manual FI items (FRIGHT vs. vFRIGHT). This suggests that the video features described herein can not only predict the relevant FI items but also contain signals for aging beyond the traditional manual FL. In addition, the detail in measurements of the features compared to FI items (using actual values rather than a simplified score of 0, 0.5, or 1) could contribute to greater performance.

Finally, using the video features as input to the random forest model, the manual FI score was predicted within 0.04±0.002 of the actual score on average. Unnormalized, this error is 1.08±0.05, which is comparable to 1 FI item being miscored by 1 point, or 2 FI items mis-scored by 0.5 points. Furthermore, simple point predictions beyond by providing 95% prediction intervals were determined. Quantile random forests were applied to low and high quantiles of the FI score's conditional distribution that revealed how certain features affected frail and healthy animals differently.

Ease of use of the trained model by non-computational labs is an important challenge. Therefore, in addition to implementation details in the Methods section, the integrated mouse phenotyping platform-a hardware and software solution—is detailed that provides tracking, pose estimation, feature generation, and automated behavior analysis in [Beane, G. et al. bioRxiv (2022)]. This platform requires a specific open field apparatus, however, researchers would be able to use the trained model if they generate the same features as the model described herein using their own open field data-collection apparatus. Any set-up that allows tracking and pose estimation using available software would allow researchers to calculate the features necessary to use our trained model.

The vFI can be further improved with the addition of new features through reanalysis of existing data and future technological improvements to data acquisition [Pereira, T. D., Shaevitz, J. W. & Murthy, M. Nature neuroscience (2020); Mathias, A. Neuron (2020)]. For instance, quantification of defecation and urination could provide information about additional systems, while higher camera quality could provide detailed information about fine motor movement-based behaviors and appearance-based features such as coat condition. Additionally, this approach could potentially be used in a long-term home cage environment. Not only would this further reduce handling and environmental factors, features such as social interaction, feeding, drinking, sleep and others could be integrated. Furthermore, given the evidence of a strong genetic component to aging [Singh, P. P., Demmitt, B. A., Nath R. D. & Brunet, A., Cell (2019)], application of this method to other strains and genetically heterogeneous populations, such as Diversity Outcross and Collaborative Cross, may reveal how genetic variation influences frailty. Further, as predicting mortality risk is a vital function of frailty, video features could be used to study lifespan. The value of this work could go beyond community adoption and toward community involvement; training data from multiple labs could provide an even more robust and accurate model. This could provide a uniform FI across studies. Overall, the approach has produced novel insights into mouse frailty and shows that video data of mouse behavior can be used to quantify aggregate abstract concepts such as frailty. The automated frailty index enables high-throughput, reliable aging studies, particularly interventional studies that are a priority for the aging research community.

### EQUIVALENTS

Although several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto; the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention. All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements, and/or steps are in any way required for one or more embodiments or that one or more embodiments necessarily include logic for deciding, with or without other input or prompting, whether these features, elements, and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

All references, patents and patent applications and publications that are cited or referred to in this application are incorporated by reference in their entirety herein.

What is claimed is:

1. A computer-implemented method comprising:
receiving video data representing a video capturing movements of a subject;
determining, using the video data, spinal mobility features of the subject for a duration of the video; and
processing, using at least one machine learning model, at least the spinal mobility features to determine a visual frailty score for the subject.

2. The computer-implemented method of claim 1, wherein determining the spinal mobility features of the subject for the duration of the video comprises:
determining a plurality of spinal measurements, each spinal measurement of the plurality of spinal measurements corresponding to one video frame of the video data; and
determining the spinal mobility features using the plurality of spinal measurements.

3. The computer-implemented method of claim 1, wherein determining the spinal mobility features of the subject for the duration of the video comprises:
for each video frame of the video data:
determining a first distance between a head of the subject and a tail of the subject;
determining a second distance between a mid-back of the subject and a midpoint between the head and the tail;
determining an angle formed between the head, the tail and the mid-back of the subject; and
determining the spinal mobility features for a video frame to include the first distance, the second distance and the angle.

4. The computer-implemented method of claim 1, wherein determining the spinal mobility features of the subject for the duration of the video comprises:
determining, for each video frame of the video data, a distance between a mid-back of the subject and a midpoint between a head of the subject and a tail of the subject.

5. The computer-implemented method of claim 1, further comprising:
processing, using at least an additional machine learning model, the video data to determine pose estimation data tracking, during the duration of the video, a location of at least a head of the subject, a tail of the subject, and a mid-back of the subject; and
using the pose estimation data to determine the spinal mobility features.

6. The computer-implemented method of claim 1, further comprising:
processing the video data to determine pose estimation data tracking, during the duration of the video, a location of at least twelve body parts of the subject;
determining, using the pose estimation data, features for the subject; and
processing, using the at least one machine learning model, the features to determine the visual frailty score.

7. The computer-implemented method of claim 1, further comprising:
determining body features for the subject, the body features corresponding to at least one of a length of the subject, a width of the subject, and a distance between rear paws of the subject;
processing, using the at least one machine learning model, the body features to determine the visual frailty score.

8. The computer-implemented method of claim 1, further comprising:
determining a number of times a rearing event occurs during the duration of the video;
determining a rearing length for each rearing event;
processing, using the at least one machine learning model, the number times the rearing event occurs and the rearing length for each rearing event to determine the visual frailty score.

9. The computer-implemented method of claim 1, further comprising:
processing, using the at least one machine learning model, the video data to determine ellipse-fit data for the subject for the duration of the video;
determining, using the ellipse-fit data, features for the subject; and
processing, using the at least one machine learning model, the features to determine the visual frailty score.

10. The computer-implemented method of claim 1, wherein determining spinal mobility features of the subject for a duration of the video comprises:
determining a first set of video frames representing gait movements by the subject;
determining a first set of spinal mobility features for the first set of video frames;
determining a second set of video frames representing non-gait movements by the subject; and
determining a second set of spinal mobility features for the second set of video frames;
wherein the spinal mobility features include the first set of spinal mobility features and the second set of spinal mobility features.

11. The computer-implemented method of claim 10, wherein the first set of spinal mobility features correspond to a distance between a mid-back of the subject and a midpoint between a head and a tail of the subject, and wherein the second set of spinal mobility features correspond to an angle formed between the head, the tail and the mid-back of the subject.

12. The computer-implemented method of claim 1, further comprising:
determining, using the video data, gait measurements of the subject for the duration of the video; and
processing, using the at least one machine learning model, the gait features to determine the visual frailty score for the subject.

13. The computer-implemented method of claim 12, further comprising:
processing the video data to determine point data tracking movement, for the duration of the video, of a set of body parts of the subject;
determining, using the point data, a plurality of stance phases and a plurality of swing phases represented in the video data;
determining, based on the plurality of stance phases and the plurality of swing phases, a plurality of stride intervals represented in the video data; and
determining, using the point data, the gait measurements based on each stride interval of the plurality of stride intervals.

14. The computer-implemented method of claim 13, further comprising:
determining a first transition from a first stance phase of the plurality of stance phases and a first swing phase of the plurality of swing phases based on a toe-off event of a left hind paw of the subject or a right hind paw of the subject;
determining a second transition from a second swing phase of the plurality of swing phases to a second stance phase of the plurality of stance phases based on a foot strike event of the left hind paw or the right hind paw; and
determining the gait measurements using the first transition and the second transition.

15. The computer-implemented method of claim 12, further comprising:
processing the video data to determine point data tracking movement, for the duration of the video, of a set of body parts of the subject, wherein the set of body parts comprises a left hind paw and a right hind paw, and wherein determining the gait measurements comprises:
determining, using the point data, a step length for a stride interval, the step length representing a distance that the right hind paw travels past a previous left hind paw strike;
determining, using the point data, a stride length using for the stride interval, the stride length representing a distance that the left hind paw travels during the each stride interval;
determining, using the point data, a step width for the stride interval, the step width representing a distance between the left hind paw and the right hind paw.

16. The computer-implemented method of claim 12, further comprising:
processing the video data to determine point data tracking movement, for the duration of the video, of a set of body parts of the subject, wherein the set of body parts comprises a tail base, and
wherein determining the gait measurements comprises determining, using the point data, speed data of the subject based on movement of the tail base for a stride interval.

17. The computer-implemented method of claim 12, further comprising:
processing the video data to determine point data tracking movement, for the duration of the video, of a set of body parts of the subject, wherein the set of body parts comprises a tail base, and
wherein determining the gait measurements comprises:
determining, using the point data, a set of speed data of the subject based on movement of the tail base during a set of frames representing a stride interval of the plurality of stride intervals; and
determining a stride speed, for the stride interval, by averaging the set of speed data.

18. The computer-implemented method of claim 12, further comprising:
processing the video data to determine point data tracking movement, for the duration of the video, of a set of body parts of the subject, wherein the set of body parts comprises a right hind paw and a left hind paw, and
wherein determining the gait measurements comprises:
determining, using the point data, first stance duration representing an amount of time that the right hind paw is in contact with ground during a stride interval;
determining a first duty factor based on the first stance duration and the duration of the stride interval;
determining, using the point data, second stance duration representing an amount of time that the left hind paw is in contact with ground during the stride interval;
determining a second duty factor based on the second stance duration and the duration of the stride interval; and
determining an average duty factor for the stride interval based on the first duty factor and the second duty factor.

19. The computer-implemented method of claim 12, further comprising:
processing the video data to determine point data tracking movement, for the duration of the video, of a set of body parts of the subject, wherein the set of body parts comprises a tail base and a neck base, and
wherein determining the gait measurements comprises:
determining, using the point data, a set of vectors connecting the tail base and the neck base during a set of frames representing a stride interval of the plurality of stride intervals; and
determining, using the set of vectors, an angular velocity of the subject for the stride interval.

20. The computer-implemented method of claim 12, further comprising:
processing the video data to determine point data tracking movement, for the duration of the video, of a set of body parts of the subject, wherein the set of body parts comprises a spine center of the subject,
wherein a stride interval is associated with a set of frames of the video data, and
wherein determining the gait measurements comprises determining, using the point data, a displacement vector for the stride interval, the displacement vector connecting the spine center represented in a first frame of the set of frames and the spine center represented in a last frame of the set of frames.

21. The computer-implemented method of claim 20, wherein the set of body parts further comprises a nose of the subject, and wherein determining the metrics data comprises:
determining, using the point data, a set of lateral displacements of the nose for the stride interval based on a perpendicular distance of the nose from the displacement vector for each frame in the set of frames.

22. The computer-implemented method of claim 21, wherein the lateral displacement of the nose is further based on a body length of the subject.

23. The computer-implemented method of claim 21, wherein determining the gait measurements further comprises determining a tail tip displacement phase offset by:
performing an interpolation using the set of lateral displacements of the nose to generate a smooth curve lateral displacement of the nose for the stride interval;
determining, using the smooth curve lateral displacement of the nose, when a maximum displacement of the nose occurs during the stride interval; and
determining a percent stride location representing a percent of the stride interval that is completed when the maximum displacement of the nose occurs.

24. The computer-implemented method of claim 12, further comprising:
processing the video data to determine point data tracking movement, for the duration of the video, of a set of body parts of the subject, wherein the set of body parts further comprises a tail base of the subject, and
wherein determining the gait measurements comprises determining, using the point data, a set of lateral displacements of the tail base for the stride interval based on a perpendicular distance of the tail base from the displacement vector for each frame in the set of frames.

25. The computer-implemented method of claim 24, wherein determining the gait measurements further comprises determining a tail base displacement phase offset by:
performing an interpolation using the set of lateral displacements of the tail base to generate a smooth curve lateral displacement of the tail base for the stride interval;
determining, using the smooth curve lateral displacement of the tail base, when a maximum displacement of the tail base occurs during the stride interval; and
determining a percent stride location representing a percent of the stride interval that is completed when the maximum displacement of the tail base occurs.

26. The computer-implemented method of claim 12, further comprising:
processing the video data to determine point data tracking movement, for the duration of the video, of a set of body parts of the subject, wherein the set of body parts comprises a tail tip of the subject, and
wherein determining the gait measurements comprises:
determining, using the point data, a set of lateral displacements of the tail tip for the stride interval based on a perpendicular distance of the tail tip from the displacement vector for each frame in the set of frames.

27. The computer-implemented method of claim 26, wherein determining the gait measurements further comprises determining a tail tip displacement phase offset by:
performing an interpolation using the set of lateral displacements of the tail tip to generate a smooth curve lateral displacement of the tail tip for the stride interval;
determining, using the smooth curve lateral displacement of the tail tip, when a maximum displacement of the tail tip occurs during the stride interval; and
determining a percent stride location representing a percent of the stride interval that is completed when the maximum displacement of the tail tip occurs.

28. The computer-implemented method of claim 1, further comprising:
processing the video data to determine point data tracking movement, for the duration of the video, of a set of body parts, wherein the set of body parts comprises one or more of:
the nose, base of neck, mid spine, left hind paw, right hind paw, base of tail, middle of tail and tip of tail;
determining, using the point data, features for the subject; and
processing, using at least the one machine learning model, the features to determine the visual frailty score.

29. The computer-implemented method of claim 1, further comprising:
processing the video data using an additional machine learning model to identify a likelihood of the subject exhibiting a grooming behavior for a plurality of video frames of the video data; and
determining the visual frailty score using the likelihood of the subject exhibiting the grooming behavior.

30. The computer-implemented method of claim 1, further comprising:
processing the video data using an additional machine learning model to identify a likelihood of the subject exhibiting a predetermined behavior for a plurality of video frames of the video data; and
determining the visual frailty score using the likelihood of the subject exhibiting the predetermined behavior.

31. The computer-implemented method of claim 1, further comprising:
determining a rotated set of video frames by rotating a first set of video frames of the video data;
processing the first set of video frames using a first machine learning model configured to identify a likelihood of the subject exhibiting a predetermined behavioral action;
based on the processing of the first set of video frames by the first machine learning model, determining a first probability of the subject exhibiting the predetermined behavioral action in a first video frame of the first set of video frames, the first video frame corresponding to a first duration of the video data;
processing the rotated set of frames using the first machine learning model;
based on the processing of the rotated set of video frames by the first machine learning model, determining a second probability of the subject exhibiting the predetermined behavioral action in a second video frame of the rotated set of video frames, the second video frame corresponding to the first duration of the video data; and
using the first probability and the second probability, identifying a first label for the first video frame, the first label indicating that the subject exhibits the predetermined behavioral action.

32. The computer-implemented method of claim 31, further comprising:
processing the first set of video frames using a second machine learning model configured to identify a likelihood of the subject exhibiting the predetermined behavioral action;
based on the processing of the first set of video frames by the second machine learning model, determining a third probability of the subject exhibiting the predetermined behavioral action in the first video frame;
processing the rotated set of video frames using the second machine learning model;
based on the processing of the rotated set of video frames by the second machine learning model, determining a fourth probability of the subject exhibiting the predetermined behavioral action in the second video frame; and
identifying the first label using the first probability, the second probability, the third probability and the fourth probability.

33. The computer-implemented method of claim 31, further comprising:
determining a reflected set of video frames by reflecting the first set of video frames;
processing the reflected set of video frames using the first machine learning model;
based on the processing of the reflected set of video frames by the first machine learning model, determining a third probability of the subject exhibiting the predetermined behavioral action in a third video frame of the reflected set of frames, the third video frame corresponding to the first duration of the first video frame; and
identifying the first label using the first probability, the second probability, and the third probability.

34. The computer-implemented method of claim 31, wherein the subject is a mouse, and the predetermined behavior comprises a grooming behavior comprising at least one of: paw licking, unilateral face wash, bilateral face wash, and flank licking.

35. The computer-implemented method of claim 31, wherein the first set of video frames represent a portion of the video data during a time period, and the first video frame is a last temporal frame of the time period.

36. The computer-implemented method of claim 31, further comprising:
identifying a second set of video frames from the video data;
determining a second rotated set of video frames by rotating the second set of video frames;
processing the second set of video frames using the first machine learning model;
based on the processing of the second set of video frames by the first machine learning model, determining a third probability of the subject exhibiting the predetermined behavioral action in a third video frame of the second set of video frames;
processing the second rotated set of video frames using the first machine learning model;
based on the processing of the second rotated set of video frames by the first machine learning model, determining a fourth probability of the subject exhibiting the predetermined behavioral action in a fourth video frame of the rotated set of frames, the fourth video frame corresponding to the third video frame; and
using the third probability and the fourth probability, identifying a second label for the fourth video frame, the second label indicating that the subject exhibits the predetermined behavioral action.

37. The computer-implemented method of claim 31, wherein the first machine learning model is a machine learning classifier.

38. The computer-implemented method of claim 1, further comprising:
processing the video data to determine gait measurements for the subject for the duration of the video;
processing the video data to determine behavior data identifying portions of the video where the subject exhibits a predetermined behavior; and
processing, using the at least one machine learning model, the spinal mobility features, the gait measurements and the behavior data to determine the visual frailty score.

39. The computer-implemented method of claim 1, wherein the video captures movements of the subject in an open field arena.

40. The computer-implemented method of claim 1, further comprising:
determining a physical condition of the subject using the visual frailty score.

41. The computer-implemented method of claim 40, wherein the physical condition is frailty.

42. The computer-implemented method of claim 40, wherein the physical condition is a pre-frailty condition.

43. The computer-implemented method of claim 1, wherein the subject is a mammal, optionally a mouse.

44. A method of assessing a physical condition of a subject, comprising determining a visual frailty score for the subject with the computer-implemented method of claim 1.

45. The method of claim 44, wherein the physical condition is frailty.

46. The method of claim 44, wherein the physical condition is a pre-frailty condition.

47. The method of claim 44, wherein the subject is a mammal, optionally a mouse.

48. A method of determining the presence of an effect of a candidate compound on a frailty condition, comprising:
obtaining a first visual frailty score for a subject, wherein a means for the obtaining comprises a computer-implemented method of claim 1, and wherein the subject has a frailty condition or is an animal model for the frailty condition;
administering to the subject the candidate compound;
obtaining a post-administration visual frailty score for the subject;
comparing the first and the post-administration visual frailty score, wherein a difference in the first and post-administration visual frailty score identifies an effect of the candidate compound on the frailty condition.

49. The method of claim 48, wherein an improvement in the visual frailty score indicating less frailty identifies the candidate compound as enhancing regression of the frailty condition.

50. The method of claim 48, wherein a post-administration visual frailty score that is statistically equivalent to the first visual frailty score identifies the candidate compound as inhibiting progression of the frailty condition in the subject.

51. The method of claim 48, further comprising additional testing of the compound's effect in treatment of the frailty condition.

52. The method of claim 48, wherein the subject is a mammal, optionally a mouse.

53. A method of identifying the presence of an effect of a candidate compound on a frailty condition, the method comprising:
administering the candidate compound to a subject that has the frailty condition or that is an animal model for the frailty condition;
obtaining a visual frailty score for the subject, wherein a means for the obtaining comprises a computer-implemented method of claim 1;
comparing the obtained visual frailty score to a control visual frailty score, wherein a difference in the obtained visual frailty score and the control visual frailty score identifies the presence of an effect of the candidate compound on the frailty condition.

54. The method of claim 53, wherein an improvement in the visual frailty score indicating less frailty in the subject administered the candidate compound compared to the control frailty score identifies the candidate compound as enhancing regression of the frailty condition in the subject.

55. The method of claim 53, wherein a visual frailty score obtained in the subject administered the candidate compound that is statistically equivalent to the control frailty score identifies the candidate compound as inhibiting progression of the frailty condition in the subject.

56. The method of claim 53, wherein the subject is a mammal, optionally a mouse.

57. A system comprising:
at least one processor; and
at least one memory comprising instructions that, when executed by the at least one processor, cause the system to:
receive video data representing a video capturing movements of a subject;
determine, using the video data, spinal mobility features of the subject for a duration of the video; and
process, using at least one machine learning model, at least the spinal mobility features to determine a visual frailty score for the subject.

58. The system of claim 57, wherein the instructions that cause the system to determine the spinal mobility features of the subject for the duration of the video further cause the system to:
determine a plurality of spinal measurements, each spinal measurement of the plurality of spinal measurements corresponding to one video frame of the video data; and
determine the spinal mobility features using the plurality of spinal measurements.

59. The system of claim 57, wherein the instructions that cause the system to determine the spinal mobility features of the subject for the duration of the video further cause the system to:
for each video frame of the video data:
determine a first distance between a head of the subject and a tail of the subject;
determine a second distance between a mid-back of the subject and a midpoint between the head and the tail;
determine an angle formed between the head, the tail and the mid-back of the subject; and
determine the spinal mobility features for a video frame to include the first distance, the second distance and the angle.

60. The system of claim 57, wherein the instructions that cause the system to determine the spinal mobility features of the subject for the duration of the video further cause the system to:
determine, for each video frame of the video data, a distance between a mid-back of the subject and a midpoint between a head of the subject and a tail of the subject.

61. The system of claim 57, wherein the at least one memory comprises further instructions, that when executed by the at least one processor, cause the system to:
process, using the at least one machine learning model, the video data to determine pose estimation data tracking, during the duration of the video, a location of at least a head of the subject, a tail of the subject, and a mid-back of the subject; and
use the pose estimation data to determine the spinal mobility features.

62. The system of claim 57, wherein the at least one memory comprises further instructions, that when executed by the at least one processor, cause the system to:
process the video data to determine pose estimation data tracking, during the duration of the video, a location of at least twelve body parts of the subject;
determine, using the pose estimation data, features for the subject; and
process, using the at least one machine learning model, the features to determine the visual frailty score.

63. The system of claim 57, wherein the at least one memory comprises further instructions, that when executed by the at least one processor, cause the system to:
determine body features for the subject, the body features corresponding to at least one of a length of the subject, a width of the subject, and a distance between rear paws of the subject;
process, using the at least one machine learning model, the body features to determine the visual frailty score.

64. The system of claim 57, wherein the at least one memory comprises further instructions, that when executed by the at least one processor, cause the system to:
determine a number of times a rearing event occurs during the duration of the video;
determine a rearing length for each rearing event;
process, using the at least one machine learning model, the number times the rearing event occurs and the rearing length for each rearing event to determine the visual frailty score.

65. The system of claim 57, wherein the at least one memory comprises further instructions, that when executed by the at least one processor, cause the system to:
process, using at least an additional machine learning model, the video data to determine ellipse-fit data for the subject for the duration of the video;
determine, using the ellipse-fit data, features for the subject; and
process, using the at least one machine learning model, the features to determine the visual frailty score.

66. The system of claim 57, wherein the instructions that cause the system to determine the spinal mobility features of the subject for the duration of the video further cause the system to:
determine a first set of video frames representing gait movements by the subject;
determine a first set of spinal mobility features for the first set of video frames;
determine a second set of video frames representing non-gait movements by the subject; and
determine a second set of spinal mobility features for the second set of video frames;
wherein the spinal mobility features include the first set of spinal mobility features and the second set of spinal mobility features.

67. The system of claim 66, wherein the first set of spinal mobility features correspond to a distance between a mid-back of the subject and a midpoint between a head and a tail of the subject, and wherein the second set of spinal mobility features correspond to an angle formed between the head, the tail and the mid-back of the subject.

68. The system of claim 57, wherein the at least one memory comprises further instructions, that when executed by the at least one processor, cause the system to:
determine, using the video data, gait measurements of the subject for the duration of the video; and
process, using the at least one machine learning model, the gait features to determine the visual frailty score for the subject.

69. The system of claim 68, wherein the at least one memory comprises further instructions, that when executed by the at least one processor, cause the system to:
process the video data to determine point data tracking movement, for the duration of the video, of a set of body parts of the subject;
determine, using the point data, a plurality of stance phases and a plurality of swing phases represented in the video data;
determine, based on the plurality of stance phases and the plurality of swing phases, a plurality of stride intervals represented in the video data; and
determine, using the point data, the gait measurements based on each stride interval of the plurality of stride intervals.

70. The system of claim 69, wherein the at least one memory comprises further instructions, that when executed by the at least one processor, cause the system to:
determine a first transition from a first stance phase of the plurality of stance phases and a first swing phase of the plurality of swing phases based on a toe-off event of a left hind paw of the subject or a right hind paw of the subject;
determine a second transition from a second swing phase of the plurality of swing phases to a second stance phase of the plurality of stance phases based on a foot strike event of the left hind paw or the right hind paw; and
determine the gait measurements using the first transition and the second transition.

71. The system of claim 68, wherein the at least one memory comprises further instructions, that when executed by the at least one processor, cause the system to:
process the video data to determine point data tracking movement, for the duration of the video, of a set of body parts of the subject, wherein the set of body parts comprises a left hind paw and a right hind paw, and wherein determining the gait measurements comprises:
determine, using the point data, a step length for a stride interval, the step length representing a distance that the right hind paw travels past a previous left hind paw strike;
determine, using the point data, a stride length using for the stride interval, the stride length representing a distance that the left hind paw travels during the each stride interval;
determine, using the point data, a step width for the stride interval, the step width representing a distance between the left hind paw and the right hind paw.

72. The system of claim 68, wherein the at least one memory comprises further instructions, that when executed by the at least one processor, cause the system to:
process the video data to determine point data tracking movement, for the duration of the video, of a set of body parts of the subject, wherein the set of body parts comprises a tail base, and
wherein the instructions that cause the system to determine the gait measurements further cause the system to determine, using the point data, speed data of the subject based on movement of the tail base for a stride interval.

73. The system of claim 68, wherein the at least one memory comprises further instructions, that when executed by the at least one processor, cause the system to:
process the video data to determine point data tracking movement, for the duration of the video, of a set of body parts of the subject, wherein the set of body parts comprises a tail base, and
wherein the instructions that cause the system to determine the gait measurements further cause the system to:
determine, using the point data, a set of speed data of the subject based on movement of the tail base during a set of frames representing a stride interval of the plurality of stride intervals; and
determine a stride speed, for the stride interval, by averaging the set of speed data.

74. The system of claim 68, wherein the at least one memory comprises further instructions, that when executed by the at least one processor, cause the system to:
process the video data to determine point data tracking movement, for the duration of the video, of a set of body parts of the subject, wherein the set of body parts comprises a right hind paw and a left hind paw, and
wherein the instructions that cause the system to determine the gait measurements further cause the system to:
determine, using the point data, first stance duration representing an amount of time that the right hind paw is in contact with ground during a stride interval;
determine a first duty factor based on the first stance duration and the duration of the stride interval;
determine, using the point data, second stance duration representing an amount of time that the left hind paw is in contact with ground during the stride interval;
determine a second duty factor based on the second stance duration and the duration of the stride interval; and
determine an average duty factor for the stride interval based on the first duty factor and the second duty factor.

75. The system of claim 68, wherein the at least one memory comprises further instructions, that when executed by the at least one processor, cause the system to:
process the video data to determine point data tracking movement, for the duration of the video, of a set of body parts of the subject, wherein the set of body parts comprises a tail base and a neck base, and
wherein the instructions that cause the system to determine the gait measurements further cause the system to:
determine, using the point data, a set of vectors connecting the tail base and the neck base during a set of frames representing a stride interval of the plurality of stride intervals; and
determine, using the set of vectors, an angular velocity of the subject for the stride interval.

76. The system of claim 68, wherein the at least one memory comprises further instructions, that when executed by the at least one processor, cause the system to:
process the video data to determine point data tracking movement, for the duration of the video, of a set of body parts of the subject, wherein the set of body parts comprises a spine center of the subject,
wherein a stride interval is associated with a set of frames of the video data, and
wherein the instructions that cause the system to determine the gait measurements further cause the system to determine, using the point data, a displacement vector for the stride interval, the displacement vector connecting the spine center represented in a first frame of the set of frames and the spine center represented in a last frame of the set of frames.

77. The system of claim 76, wherein the set of body parts further comprises a nose of the subject, and wherein the instructions that cause the system to determine the metrics data further cause the system to:
determine, using the point data, a set of lateral displacements of the nose for the stride interval based on a perpendicular distance of the nose from the displacement vector for each frame in the set of frames.

78. The system of claim 77, wherein the lateral displacement of the nose is further based on a body length of the subject.

79. The system of claim 77, wherein the instructions that cause the system to determine the gait measurements further cause the system to determine a tail tip displacement phase offset by:
performing an interpolation using the set of lateral displacements of the nose to generate a smooth curve lateral displacement of the nose for the stride interval;
determining, using the smooth curve lateral displacement of the nose, when a maximum displacement of the nose occurs during the stride interval; and
determining a percent stride location representing a percent of the stride interval that is completed when the maximum displacement of the nose occurs.

80. The system of claim 68, wherein the at least one memory comprises further instructions, that when executed by the at least one processor, cause the system to:
process the video data to determine point data tracking movement, for the duration of the video, of a set of body parts of the subject, wherein the set of body parts further comprises a tail base of the subject, and
wherein the instructions that cause the system to determine the gait measurements further cause the system to determine, using the point data, a set of lateral displacements of the tail base for the stride interval based on a perpendicular distance of the tail base from the displacement vector for each frame in the set of frames.

81. The system of claim 80, wherein the instructions that cause the system to determine the gait measurements further cause the system to determine a tail base displacement phase offset by:
performing an interpolation using the set of lateral displacements of the tail base to generate a smooth curve lateral displacement of the tail base for the stride interval;
determining, using the smooth curve lateral displacement of the tail base, when a maximum displacement of the tail base occurs during the stride interval; and
determining a percent stride location representing a percent of the stride interval that is completed when the maximum displacement of the tail base occurs.

82. The system of claim 68, wherein the at least one memory comprises further instructions, that when executed by the at least one processor, cause the system to:
process the video data to determine point data tracking movement, for the duration of the video, of a set of body parts of the subject, wherein the set of body parts comprises a tail tip of the subject, and
wherein the instructions that cause the system to determine the gait measurements further cause the system to:
determine, using the point data, a set of lateral displacements of the tail tip for the stride interval based on a perpendicular distance of the tail tip from the displacement vector for each frame in the set of frames.

83. The system of claim 82, wherein the instructions that cause the system to determine the gait measurements further cause the system to determine a tail tip displacement phase offset by:
performing an interpolation using the set of lateral displacements of the tail tip to generate a smooth curve lateral displacement of the tail tip for the stride interval;
determining, using the smooth curve lateral displacement of the tail tip, when a maximum displacement of the tail tip occurs during the stride interval; and
determining a percent stride location representing a percent of the stride interval that is completed when the maximum displacement of the tail tip occurs.

84. The system of claim 57, wherein the at least one memory comprises further instructions, that when executed by the at least one processor, cause the system to:
process the video data to determine point data tracking movement, for the duration of the video, of a set of body parts, wherein the set of body parts comprises one or more of the nose, base of neck, mid spine, left hind paw, right hind paw, base of tail, middle of tail and tip of tail;
determine, using the point data, features for the subject; and
process, using at least the one machine learning model, the features to determine the visual frailty score.

85. The system of claim 57, wherein the at least one memory comprises further instructions, that when executed by the at least one processor, cause the system to:
process the video data using an additional machine learning model to identify a likelihood of the subject exhibiting a grooming behavior for a plurality of video frames of the video data; and
determine the visual frailty score using the likelihood of the subject exhibiting the grooming behavior.

86. The system of claim 57, wherein the at least one memory comprises further instructions, that when executed by the at least one processor, cause the system to:
process the video data using an additional machine learning model to identify a likelihood of the subject exhibiting a predetermined behavior for a plurality of video frames of the video data; and
determine the visual frailty score using the likelihood of the subject exhibiting the predetermined behavior.

87. The system of claim 57, wherein the at least one memory comprises further instructions, that when executed by the at least one processor, cause the system to:
determine a rotated set of video frames by rotating a first set of video frames of the video data;
process the first set of video frames using a first machine learning model configured to identify a likelihood of the subject exhibiting a predetermined behavioral action;
based on the processing of the first set of video frames by the first machine learning model, determine a first probability of the subject exhibiting the predetermined behavioral action in a first video frame of the first set of video frames, the first video frame corresponding to a first duration of the video data;
process the rotated set of frames using the first machine learning model;
based on the processing of the rotated set of video frames by the first machine learning model, determine a second probability of the subject exhibiting the predetermined behavioral action in a second video frame of the rotated set of video frames, the second video frame corresponding to the first duration of the video data; and
use the first probability and the second probability, identifying a first label for the first video frame, the first label indicating that the subject exhibits the predetermined behavioral action.

88. The system of claim 87, wherein the at least one memory comprises further instructions, that when executed by the at least one processor, cause the system to:
process the first set of video frames using a second machine learning model configured to identify a likelihood of the subject exhibiting the predetermined behavioral action;
based on the processing of the first set of video frames by the second machine learning model, determine a third probability of the subject exhibiting the predetermined behavioral action in the first video frame;
process the rotated set of video frames using the second machine learning model;
based on the processing of the rotated set of video frames by the second machine learning model, determine a fourth probability of the subject exhibiting the predetermined behavioral action in the second video frame; and
identify the first label using the first probability, the second probability, the third probability and the fourth probability.

89. The system of claim 87, wherein the at least one memory comprises further instructions, that when executed by the at least one processor, cause the system to:
determine a reflected set of video frames by reflecting the first set of video frames;
process the reflected set of video frames using the first machine learning model;
based on the processing of the reflected set of video frames by the first machine learning model, determine a third probability of the subject exhibiting the predetermined behavioral action in a third video frame of the reflected set of frames, the third video frame corresponding to the first duration of the first video frame; and
identify the first label using the first probability, the second probability, and the third probability.

90. The system of claim 87, wherein the subject is a mouse, and the predetermined behavior comprises a grooming behavior comprising at least one of: paw licking, unilateral face wash, bilateral face wash, and flank licking.

91. The system of claim 87, wherein the first set of video frames represent a portion of the video data during a time period, and the first video frame is a last temporal frame of the time period.

92. The system of claim 87, wherein the at least one memory comprises further instructions, that when executed by the at least one processor, cause the system to:
identify a second set of video frames from the video data;
determine a second rotated set of video frames by rotating the second set of video frames;
process the second set of video frames using the first machine learning model;
based on the processing of the second set of video frames by the first machine learning model, determine a third probability of the subject exhibiting the predetermined behavioral action in a third video frame of the second set of video frames;
process the second rotated set of video frames using the first machine learning model;
based on the processing of the second rotated set of video frames by the first machine learning model, determine a fourth probability of the subject exhibiting the predetermined behavioral action in a fourth video frame of the rotated set of frames, the fourth video frame corresponding to the third video frame; and
use the third probability and the fourth probability, identifying a second label for the fourth video frame, the second label indicating that the subject exhibits the predetermined behavioral action.

93. The system of claim 87, wherein the first machine learning model is a machine learning classifier.

94. The system of claim 57, wherein the at least one memory comprises further instructions, that when executed by the at least one processor, cause the system to:
process the video data to determine gait measurements for the subject for the duration of the video;
process the video data to determine behavior data identifying portions of the video where the subject exhibits a predetermined behavior; and
process, using the at least one machine learning model, the spinal mobility features, the gait measurements and the behavior data to determine the visual frailty score.

95. The system of claim 57, wherein the video captures movements of the subject in an open field arena.

96. The system of claim 57, wherein the at least one memory comprises further instructions, that when executed by the at least one processor, cause the system to:
determine a physical condition of the subject using the visual frailty score.

97. The system of claim 96, wherein the physical condition is frailty.

98. The system of claim 96, wherein the physical condition is a pre-frailty condition.

99. The system of claim 57, wherein the subject is a mammal, optionally a mouse.
